(19) United States
(12) Patent Application Publication
(10) Pub. No.: US 2022/0220089 A1
(43) Pub. Date: Jul. 14, 2022

(54) CATALYTIC CANNABINOID PROCESSES AND PRECURSORS

(71) Applicant: KARE CHEMICAL TECHNOLOGIES INC., Mississauga (CA)

(72) Inventors: Kamaluddin ABDUR-RASHID, Mississauga (CA); Wenli JIA, Toronto (CA); Kareem ABDUR-RASHID, Mississauga (CA)

(21) Appl. No.: 17/613,936

(22) PCT Filed: May 20, 2020
(86) PCT No.: PCT/CA2020/050674

§ 371 Date: Nov. 23, 2021

Related U.S. Application Data
(60) Provisional application No. 62/890,661, filed on Aug. 23, 2019.
(60) Provisional application No. 62/851,837, filed on May 23, 2019.

(51) Int. Cl. C07D 311/78; C07C 35/21; C07C 37/50; C07C 43/215; C07F 5/02; C07F 7/18

(52) U.S. Cl. CPC C07D 311/78; C07C 35/21; C07C 37/50; C07F 7/1804; C07F 5/027; C07F 7/188; C07C 43/215

(57) ABSTRACT

The present disclosure relates to new cannabinoid sulfonate esters and processes for their use to prepare cannabinoids. The disclosure also relates to the use of catalysts and catalytic processes for the preparation of cannabinoids from the cannabinoid sulfonate esters.

### CROSS-REFERENCE TO RELATED APPLICATIONS

This application claims the benefit of priority to U.S. Provisional Application Nos. 62/851,837, filed May 23, 2019, and 62/890,661, filed Aug. 23, 2019, the contents of which are incorporated herein by reference in their entirety.

### FIELD OF THE DISCLOSURE

The present disclosure relates to cannabinoid sulfonate ester compounds and the use of the compounds for the preparation of cannabinoids. The disclosure also relates to the use of catalysts and catalytic processes for the preparation of cannabinoids using the cannabinoid sulfonate esters as precursors.

### BACKGROUND OF THE DISCLOSURE

Cannabidiol (CBD) is the non-psychoactive and primary medicinal component of the cannabis plant. As such, CBD has significant medicinal benefits. It has been shown to counteract the psychoactive effect of tetrahydrocannabinol (THC); the other main component of cannabis. Hence, over the years a variety of CBD-rich strains of cannabis has been developed and used medicinally for treating inflammation, AIDS, ALS, Alzheimer's disease, anorexia, anxiety, arthritis, asthma, cancer, depression, diabetes, epilepsy, glaucoma, migraine, nausea, neuropathic pain, Parkinson's disease, just to name a few. In addition, there are numerous clinical trials being conducted worldwide for pharmaceutical applications of CBD, THC, Cannabidivarin (CBDV), Tetrahydrocannabivarin (THV) and other cannabinoids for these and numerous other illnesses.

The demand for pure, single component CBD and other cannabinoids is growing rapidly and as the demand for medicinal and legal recreational cannabis continues to grow, the amount of cannabis plants grown and harvested specifically for the extraction of cannabinoids will diminish. The advantage of synthesized cannabinoids relative to the products extracted from medicinal cannabis or hemp plants is the stability of supply, and control over quality and scalability. The output can always be adjusted depending on demand. Extracted cannabis resin contains more than 150 cannabinoid products, in addition to other compounds present in the plant. Even for cannabis plants with high CBD or THC content, the process of extracting and purifying the products is laborious, time consuming and only small amounts of the desired components relative to the amount of plant material is realized. In addition, the cannabis or hemp crop and quality can be impacted by drought, pests, pesticides and inclement weather.

Hence, researchers have developed or are actively developing processes for biologically derived (Luo et al. Nature 2019, 567, 123-126) or chemically synthesized cannabinoid products. Various synthetic approaches for single component cannabinoid products have been described in the prior art, each reflecting the specialization of the researcher, or the objective of a company or sponsor.

Several groups have reported the acid catalyzed alkylation of olivetol with menthadienol (US 2007/0072939). However, this procedure leads to a mixture of products which have to be tediously separated and purified using chromatography.

One group reported the Lewis acid catalyzed preparation of cannabidiolic acid esters from carboxylic acid ester derivatives of olivetol and menthadienol (EP 2578561; U.S. Pat. No. 7,674,922). Cannabidiol was subsequently obtained after hydrolysis and decarboxylation. The yields are low relative to the starting materials, and the use of costly precious metal catalysts makes the process expensive.

Another group reported the use of acid catalyzed alkylation of dihalide derivatives of olivetol and related compounds with menthadienol (Srebnik et al. J. Chem. Soc. Perkin Trans. 1987, 1423-1427; U.S. Pat. No. 10,059,683). However, the procedure is laborious and tedious, since it requires time consuming steps for halogenation of the precursor and dehalogenation of the products.

Other researchers have explored the use of chiral total synthesis procedures (Kobayashi et al. Org. Lett. 2006, 8, 2699-2702; Carreira et al. J. Am. Chem. Soc. 2017, 139, 18206-18212). However, these have limited scope due to the difficulties in obtaining the desired chiral precursors and products in high yields and purities.

The prior art reflects the difficulties associated with developing reliable and commercially viable routes for synthetic cannabinoids. This is partly due to the nature of the products, which are difficult to crystalize and separate from each other. There is a need for a better process for developing synthetic cannabinoids.

### SUMMARY OF THE DISCLOSURE

The present invention, in some aspects, describes an approach to developing synthetic cannabinoids that focuses on the use of cheap and commercially available chemicals and use of these chemicals to prepare stable precursors that can be transformed into the desired cannabinoid product on demand. Such commercially available chemicals include, but are not limited to limonene, resorcinol and their derivatives.

In various aspects, the invention relates to the preparation of new cannabinoid sulfonate ester compounds and the use of such sulfonate ester compounds for the preparation of cannabinoid products using catalysts and catalytic processes to substitute the sulfonate groups. The cannabinoid sulfonate esters can be prepared and purified prior to transformation to the desired individual cannabinoid products. The cannabinoid sulfonate esters are air-stable and shelf-stable compounds that can be stored, transported and converted into the desired cannabinoid products on demand.

Accordingly, in some embodiments, the present invention relates to cannabinoid sulfonate esters of Formula (I):

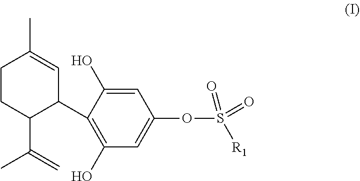

wherein, R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group. In a general way, the compounds of Formula (I) can be prepared and isolated prior to use.

In some other aspects, the present disclosure also relates to cannabinoid sulfonate esters of Formula (II):

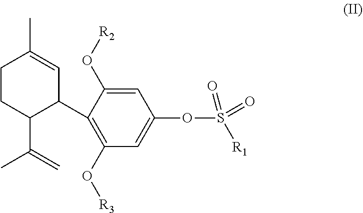

wherein, R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group;

and R₂ and R₃ represents a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more groups. In a general way, the compounds of Formula (II) can be prepared and isolated prior to use.

In various embodiments of the invention, the transformations to which the compounds of the invention can be applied include but are not limited to catalytic and non-catalytic carbon-carbon bond forming reactions including Ullman, Suzuki-Miyaura, Negishi, Kumada, Sonogashira and Stille reactions. Such carbon-carbon bond forming reactions include the use of compounds of the present disclosure, such as those of Formula (I) and (II) to prepare one or more of the cannabinoid compounds selected from the group consisting of:

### Formula (III):

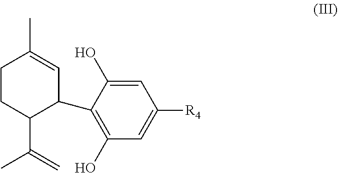

### and Formula (IV):

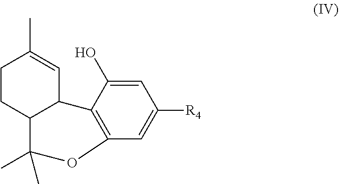

### and Formula (V):

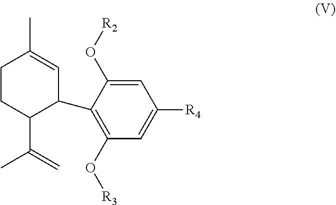

### and Formula (VI):

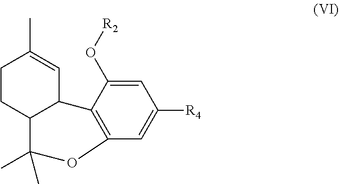

wherein, R₂ and R₃ represents a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more groups;
and R₄ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

In some other aspects of the invention, the present invention provides a method for the synthesis of one or more of the cannabinoid products below:

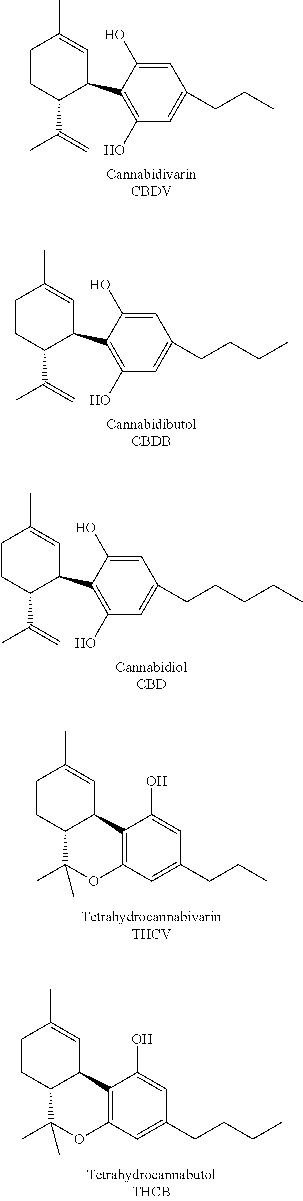 [SMILES: C=C(C)[C@@H]1CCC(C)=C[C@H]1c1c(O)cc(CCC)cc1O ; C=C(C)[C@@H]1CCC(C)=C[C@H]1c1c(O)cc(CCCC)cc1O ; C=C(C)[C@@H]1CCC(C)=C[C@H]1c1c(O)cc(CCCCC)cc1O ; CCCc1cc(O)c2c(c1)OC(C)(C)[C@@H]1CCC(C)=C[C@@H]21 ; CCCCc1cc(O)c2c(c1)OC(C)(C)[C@@H]1CCC(C)=C[C@@H]21]

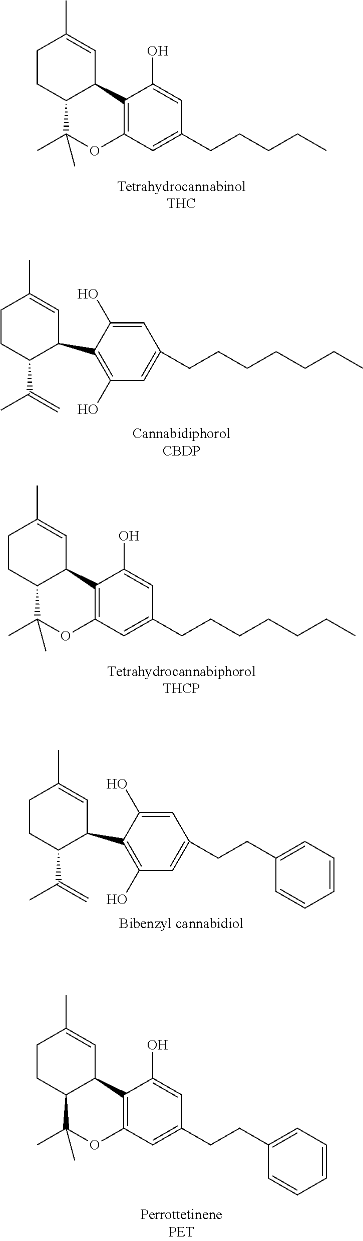 [SMILES: CCCCCc1cc(O)c2c(c1)OC(C)(C)[C@@H]1CCC(C)=C[C@@H]21 ; C=C(C)[C@@H]1CCC(C)=C[C@H]1c1c(O)cc(CCCCCCC)cc1O ; CCCCCCCc1cc(O)c2c(c1)OC(C)(C)[C@@H]1CCC(C)=C[C@@H]21 ; C=C(C)[C@@H]1CCC(C)=C[C@H]1c1c(O)cc(CCc2ccccc2)cc1O ; CC1=C[C@H]2c3c(O)cc(CCc4ccccc4)cc3OC(C)(C)[C@H]2CC1]

In some aspects the invention provides a process for the catalytic preparation of a compound of Formula (III), Formula (IV), Formula (V) or Formula (VI) from a compound of Formula (I) or Formula (II). In some other aspects the invention provides a process for the non-catalytic preparation of a compound of Formula (III), Formula (IV), Formula (V) or Formula (VI) from a compound of Formula (I) or Formula (II). In various embodiments, the process for the preparation of a compound of Formula (III), Formula (IV), Formula (V) or Formula (VI) from a compound of Formula (I) or Formula (II) pursuant to the invention uses a boron containing compound such as R₄—B(OH)₂, R₄—B(OR)₂ or R₄—BF₃K. In some other aspects of the process of the invention a Grignard compound such as R₄—MgX is used to prepare Formula (III), Formula (IV), Formula (V) or Formula (VI). In still other aspects of the process of the invention an organozinc compound such as R₄—ZnX is used to prepare Formula (III), Formula (IV), Formula (V) or Formula (VI).

In some aspects, the invention provides a compound or composition comprising: Formula (III), Formula (IV), Formula (V) or Formula (VI) where the compounds, or compositions as the case may be pure isomers or a mixture of isomers.

In some other aspects, the compounds and compositions of the invention comprise all isomers of compounds of Formula (I) and Formula (II). In some other embodiments it provides a mixture of isomers of compounds of Formula (I) and Formula (II). In yet some other embodiment it provides single isomers of compounds of Formula (I) and Formula (II). In some other aspects, the invention provides processes and methods for producing any of the foregoing.

The present invention also includes, compositions, methods of producing the compound and compositions comprising the compounds of the invention, kits comprising any one or more of the components of the foregoing, optionally with instructions to make or use same and uses of any of the foregoing.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The invention will be described in greater detail with reference to the following drawings in which, which are meant to be illustrative by certain embodiments of the invention and are not meant to limit the scope of the invention:

FIG. 1 shows the scheme for the preparation of cannabidiol (CBD);

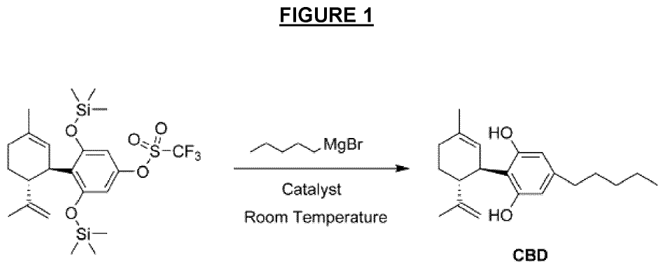

FIG. 2 shows the X-ray crystal structure of 2-((1R,6R)-3-methyl-6-(prop-1-en-2-yl)cyclohex-2-enyl)benzene-1,3,5-triol;

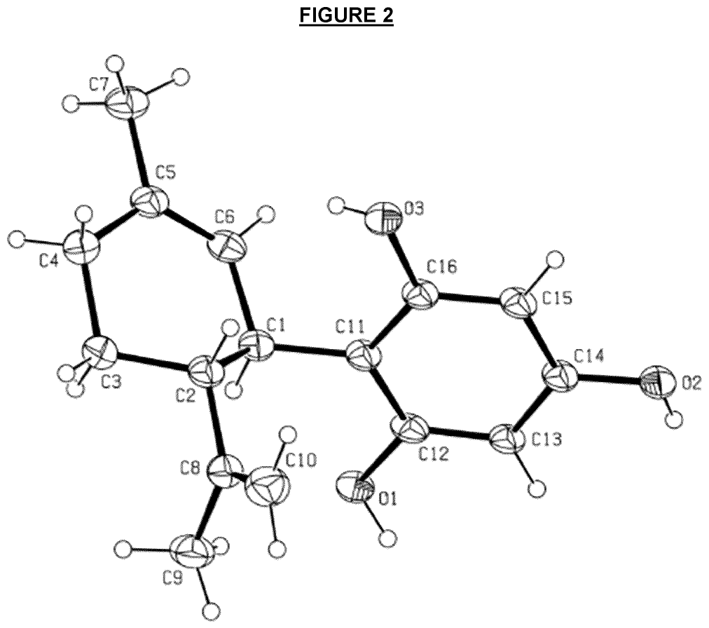

FIG. 3 shows the X-ray crystal structure of 3,5-dihydroxy-4-((1R,6R)-3-methyl-6-(prop-1-en-2-yl)cyclohex-2-enyl)phenyl trifluoromethanesulfonate;

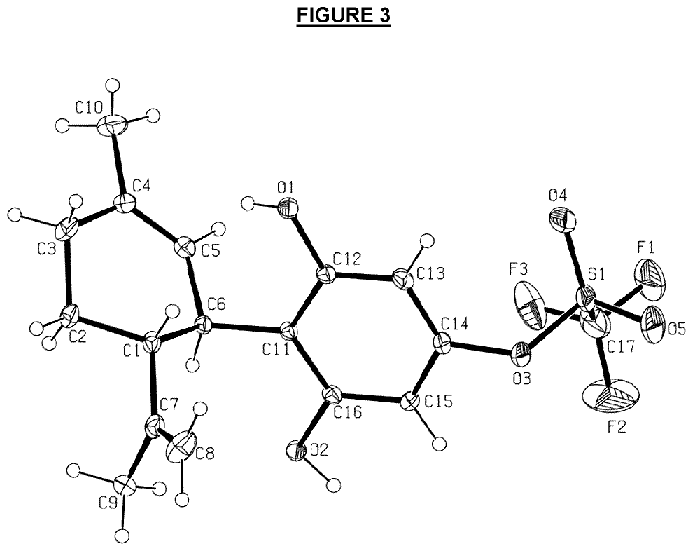

FIG. 4 shows the X-ray crystal structure of Cannabidiol;

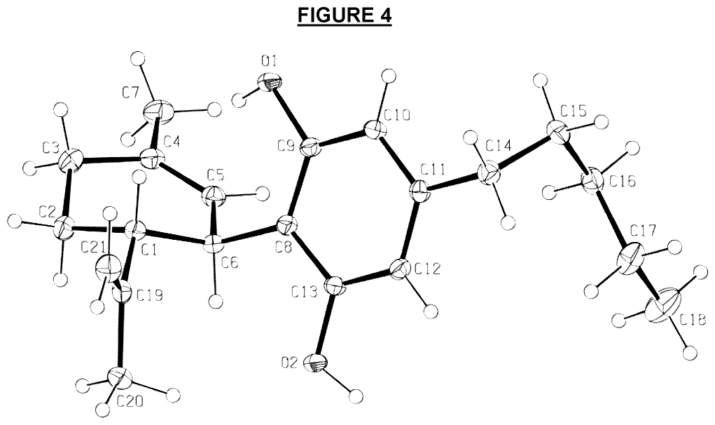

FIG. 5 shows the 1H NMR spectrum of 2-((1R,6R)-3-methyl-6-(prop-1-en-2-yl)cyclohex-2-enyl)benzene-1,3,5-triol;

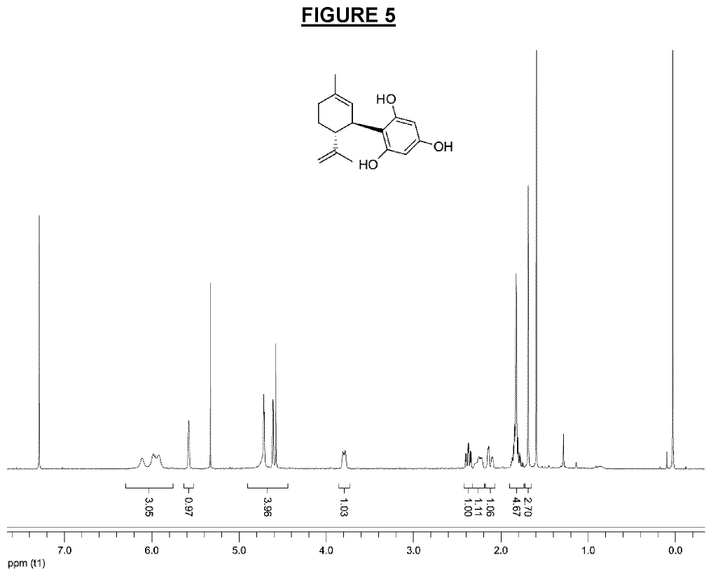

FIG. 6 shows the 1H NMR spectrum of 3,5-dihydroxy-4-((1R,6R)-3-methyl-6-(prop-1-en-2-yl)cyclohex-2-enyl)phenyl trifluoromethanesulfonate;

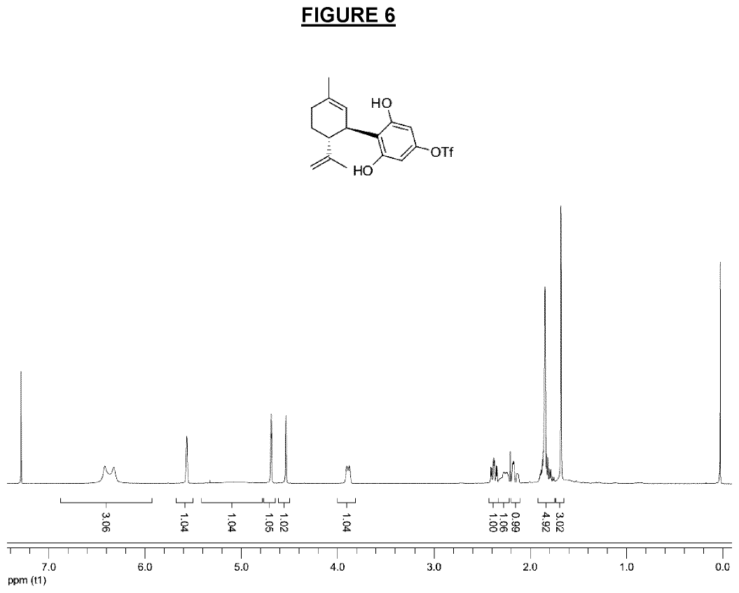

FIG. 7 shows the 1H NMR spectrum of 44(1R,6R)-3-methyl-6-(prop-1-en-2-yl)cyclohex-2-enyl)-3,5-bis(trimethylsilyloxy)phenyl trifluoromethanesulfonate;

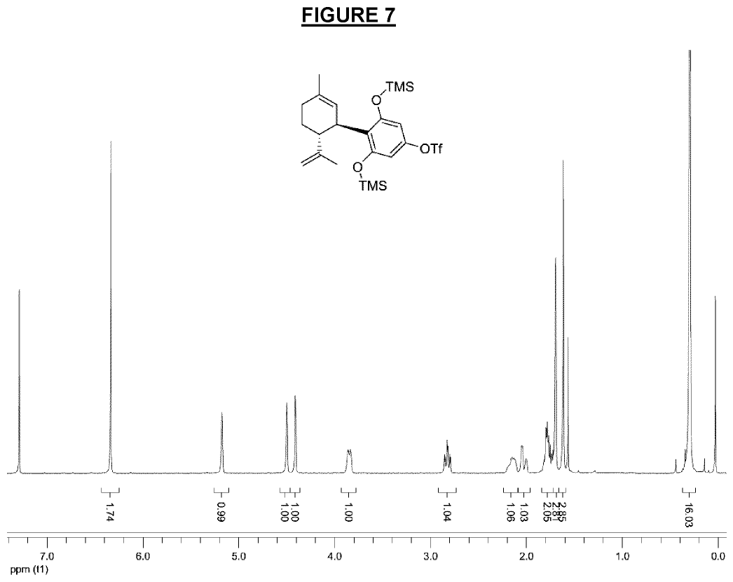

FIG. 8 shows the 1H NMR spectrum of Cannabidiol (CBD);

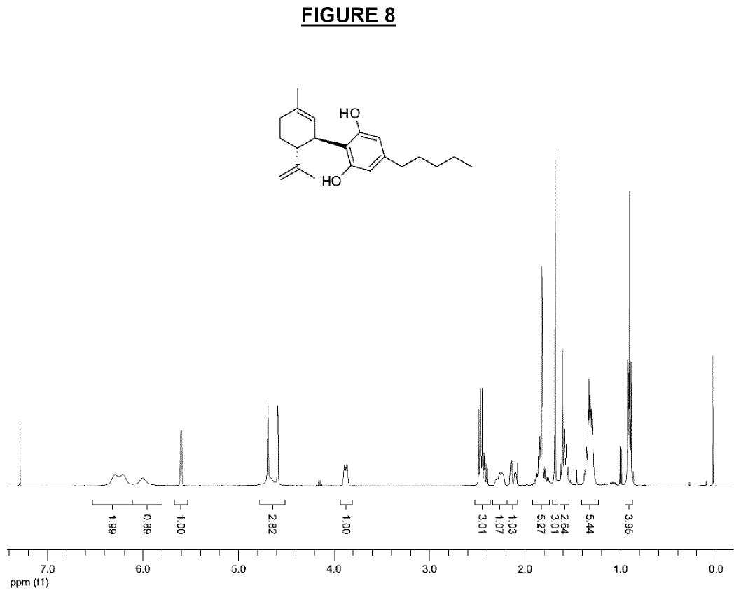

FIG. 9 shows the 1H NMR spectrum of Tetrahydrocannabinol (THC);

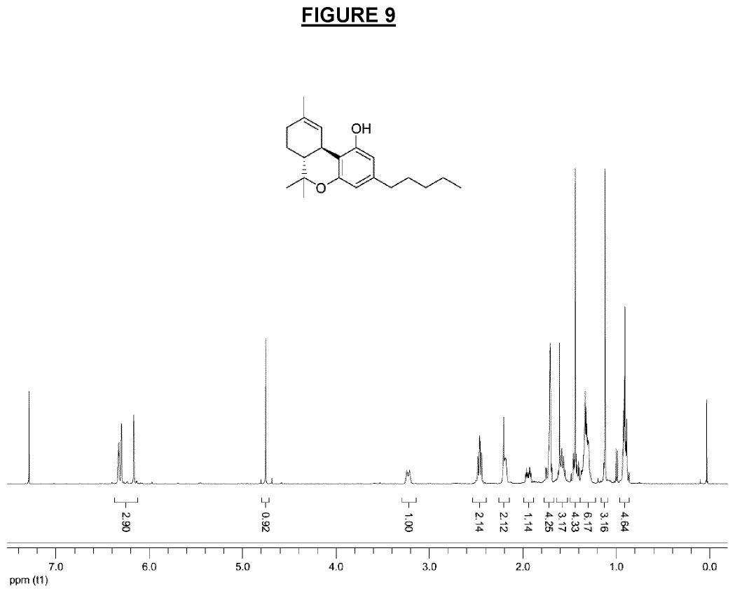

FIG. 10 shows the 1H NMR spectrum of (5-heptyl-2-((1R,6R)-3-methyl-6-(prop-1-en-2-yl)cyclohex-2-enyl)-1,3-phenylene)bis(oxy)bis(trimethylsilane);

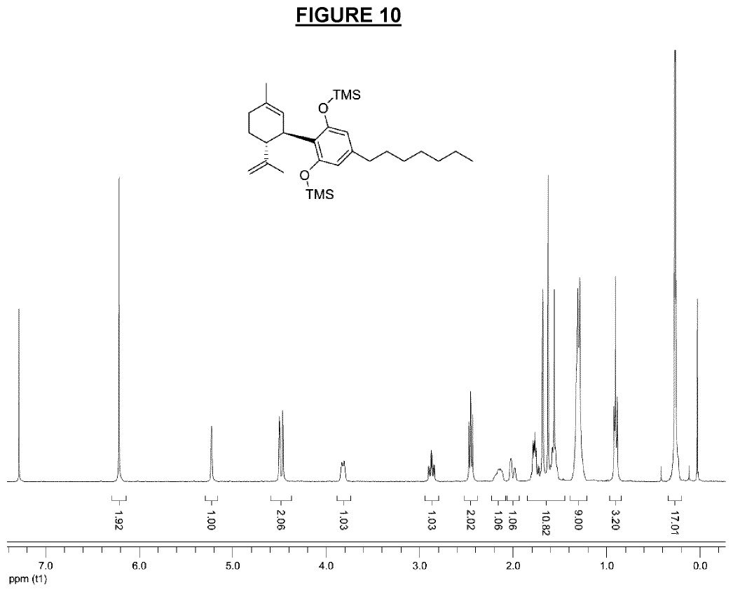

FIG. 11 shows the 1H NMR spectrum of Cannabidiphorol (CBDP);

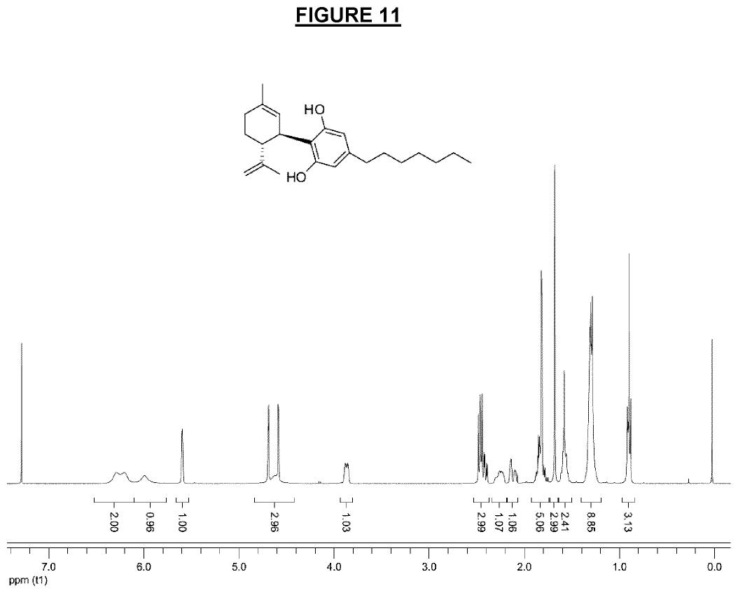

FIG. 12 shows the 1H NMR spectrum of Tetrahydrocannabiphorol (THCP);

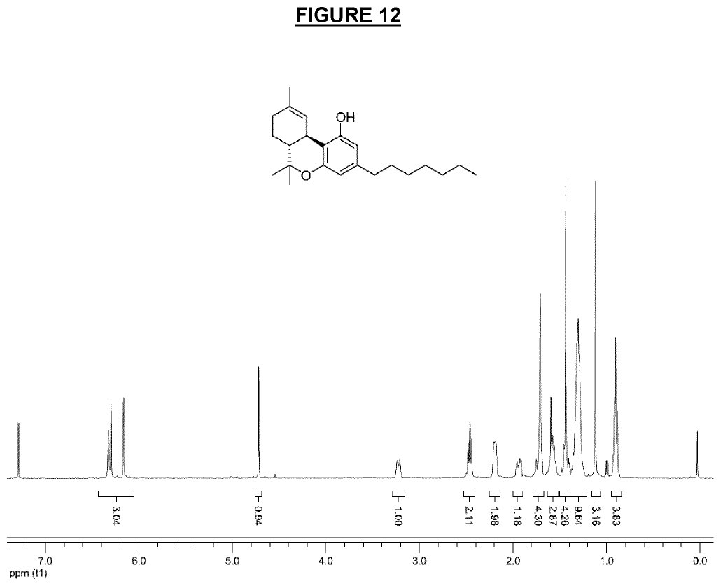

FIG. 13 shows the X-ray crystal structure of 2-((1S,6R)-3-methyl-6-(prop-1-en-2-yl)cyclohex-2-enyl)benzene-1,3,5-triol;

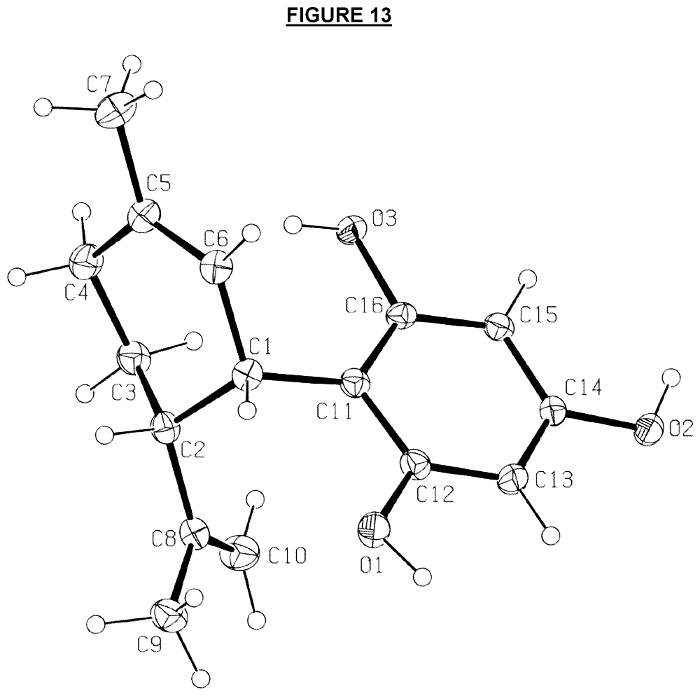

FIG. 14 shows the 1H NMR spectrum of 2-((1S,6R)-3-methyl-6-(prop-1-en-2-yl)cyclohex-2-enyl)benzene-1,3,5-triol; and

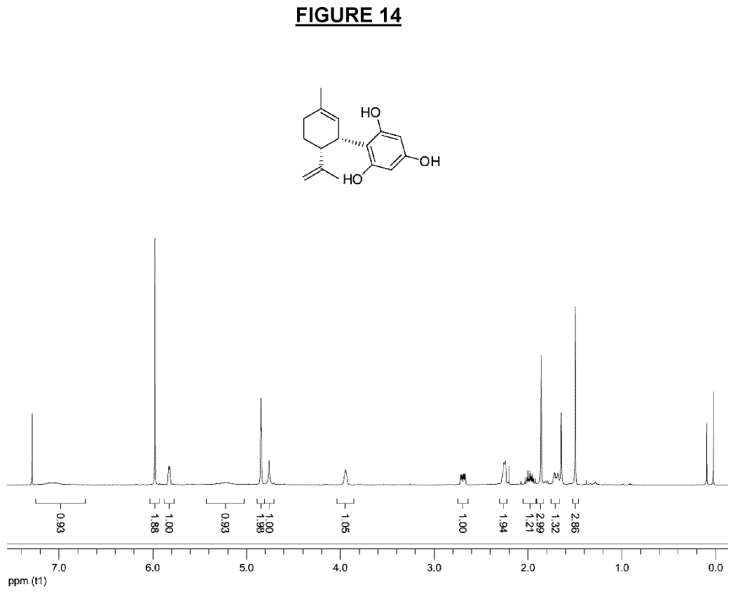

FIG. 15 shows the 1H NMR spectrum of 4-((1S,6R)-3-methyl-6-(prop-1-en-2-yl)cyclohex-2-enyl)-3,5-bis(trimethylsilyloxy)phenyl trifluoromethanesulfonate.

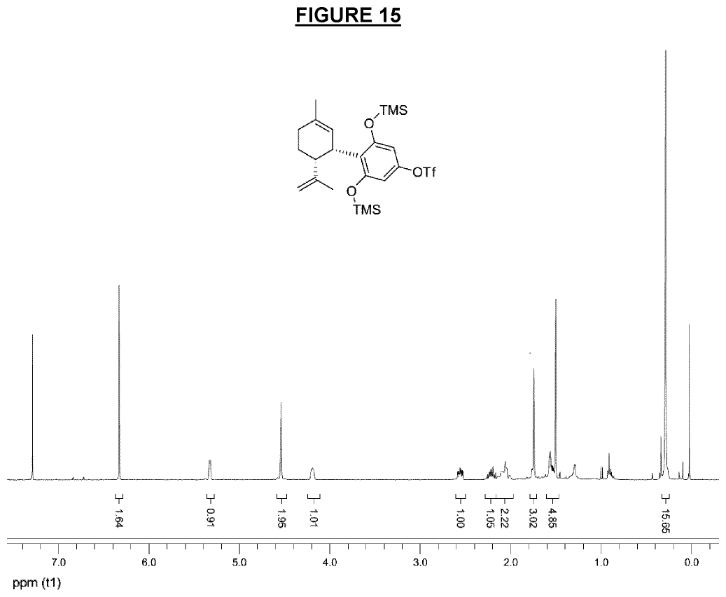

### DETAILED DESCRIPTION OF THE DISCLOSURE

### (I) Definitions

The term “alkyl” as used herein means straight and/or branched chain, saturated alkyl radicals containing one or more carbon atoms and includes (depending on the identity) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like.

The term “alkenyl” as used herein means straight and/or branched chain, unsaturated alkyl radicals containing two or more carbon atoms and one to three double bonds, and includes (depending on the identity) vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like.

The term “alkynyl” as used herein means straight and/or branched chain, unsaturated alkyl radicals containing two or more carbon atoms and one to three triple bonds, and includes (depending on the identity) acetylynyl, propynyl, but-1-ynyl, but-2-ynyl, but-3-ynyl, 3-methylbut-1-enyl, 3-methylpent-1-ynyl, 4-methylpent-1-ynyl, 4-methylpent-2-ynyl, penta-1,3-di-ynyl, hexyn-1-yl and the like.

The term “alkoxy” as used herein means straight and/or branched chain alkoxy group containing one or more carbon atoms and includes (depending on the identity) methoxy, ethoxy, propyloxy, isopropyloxy, t-butoxy, heptoxy, and the like.

The term “cycloalkyl” as used herein means a monocyclic, bicyclic or tricyclic saturated carbocylic group containing three or more carbon atoms and includes (depending on the identity) cyclopropyl, cyclobutyl, cyclopentyl, cyclodecyl and the like.

The term “aryl” as used herein means a monocyclic, bicyclic or tricyclic aromatic ring system containing at least one aromatic ring and 6 or more carbon atoms and includes phenyl, naphthyl, anthracenyl, 1,2-dihydronaphthyl, 1,2,3,4-tetrahydronaphthyl, fluorenyl, indanyl, indenyl and the like.

The term “heteroaryl” as used herein means a monocyclic, bicyclic or tricyclic ring system containing one or two aromatic rings and 5 or more atoms of which, unless otherwise specified, one, two, three, four or five are heteromoieties independently selected from N, NH, N(alkyl), O and S and includes thienyl, furyl, pyrrolyl, pyridinyl, indolyl, quinolyl, isoquinolyl, tetrahydroquinolyl, benzofuryl, benzothienyl and the like.

The term “halo” or “halogen” as used herein means chloro, fluoro, bromo or iodo.

The term “fluoro-substituted” as used herein means that at least one, including all, of the hydrogens on the referenced group is replaced with fluorine.

The suffix “ene” added on to any of the above groups means that the group is divalent, i.e. inserted between two other groups.

The term “ring system” as used herein refers to a carbon-containing ring system, that includes monocycles, fused bicyclic and polycyclic rings, bridged rings and metallocenes. Where specified, the carbons in the rings may be substituted or replaced with heteroatoms.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. For instance, “including” also encompasses “including but not limited to”. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### (II) Compounds of the Disclosure

The present disclosure relates to cannabinoid sulfonate esters of Formula (I) and any stereoisomers or acceptable salts thereof:

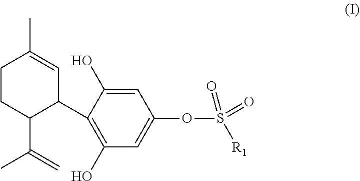

wherein, R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group. In a general way, the compounds of Formula (I) can be prepared and isolated prior to use.

In one embodiment, R₁ represents a hydrogen atom, —OR^{c}, —NR^{c}₂, fluoro-substituted-(C₁-C₂₀)-alkyl, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, or a (C₅-C₁₄)-heteroaryl group, wherein the latter 6 groups are each optionally substituted with one or more halogen atoms (F, Cl, Br or I), —(C₁-C₂₀)-alkyl, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

In another embodiment, R₁ represents a hydrogen atom, fluoro-substituted-(C₁-C₂₀)-alkyl, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, a (C₅-C₁₄)-heteroaryl group, wherein the latter 6 groups are each optionally substituted with one or more halogen atoms (F, Cl, Br or I), —(C₁-C₂₀)-alkyl, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

In another embodiment, R₁ represents a hydrogen atom, fluoro-substituted-(C₁-C₁₀)-alkyl, a (C₁-C₁₀)-alkyl group, a (C₂-C₁₀)-alkenyl group, a (C₂-C₁₀)-alkynyl group, a (C₃-C₁₀)-cycloalkyl group, a (C₆-C₁₀)-aryl group, a (C₅-C₁₀)-heteroaryl group, wherein the latter 6 groups are each optionally substituted with one or more halogen atoms (F, Cl, Br or I), —(C₁-C₂₀)-alkyl, a (C₂-C₂₀)-alkenyl group, or a (C₂-C₂₀)-alkynyl group.

In another embodiment, R₁ represents a hydrogen atom, fluoro-substituted-(C₁-C₆)-alkyl, a (C₁-C₆)-alkyl group, a (C₂-C₆)-alkenyl group, a (C₂-C₆)-alkynyl group, a (C₃-C₆)-cycloalkyl group, a (C₆)-aryl group, a (C₅-C₆)-heteroaryl group, wherein the latter 6 groups are each optionally substituted with one or more halogen atoms (F, Cl, Br or I), or —(C₁-C₂₀)-alkyl.

In another embodiment, R₁ represents a hydrogen atom, fluoro-substituted-(C₁-C₆)-alkyl, a (C₁-C₆)-alkyl group, or a phenyl group, wherein the latter 2 groups are each optionally substituted with one or more halogen atoms (F, Cl, Br or I), or —(C₁-C₁₀)-alkyl.

In another embodiment, R₁ represents a hydrogen atom, —CF₃,

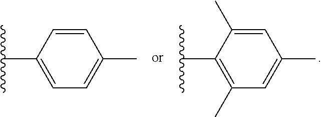

In one embodiment, the compound of Formula (I) is

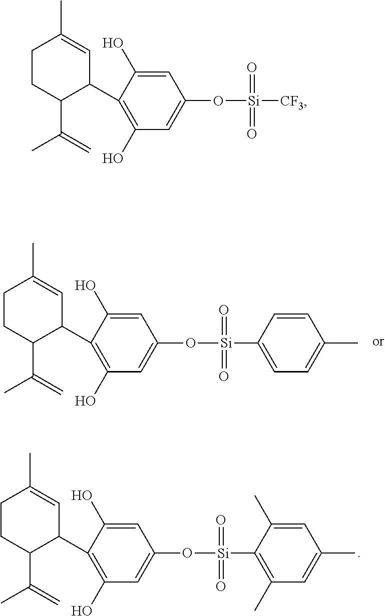

In one embodiment, the compound of the Formula (I) is a compound of Formula (IA)

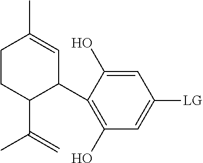

wherein LG is any suitable leaving group, such as a halo group, sulphonates, or boronates. In another embodiment, the boronate leaving group is —B(OR)₂, where R is H, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₄)-aryl group. In another embodiment, the boronate leaving group is —B(OR)₂, where R is H, a (C₁-C₂₀)-alkyl group (such as a (C₁-C₁₀)-alkyl group) or a (C₆-C₁₄)-aryl group (such as a (C₆-C₁₀)-aryl group). In another embodiment, the boronate leaving group is —BF₃K. In another embodiment, the leaving group is a triflate, mesylate or tosylate group.

The present disclosure also relates to cannabinoid sulfonate esters of Formula (II) and any stereoisomers or acceptable salts thereof:

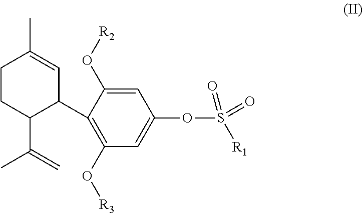

wherein, R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group;
and R₂ and R₃ represents a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more groups. In a general way, the compounds of Formula (II) can be prepared and isolated prior to use.

In another embodiment, R₁ in the compound of Formula (II) is as defined in all embodiments for the compound of Formula (I).

In one embodiment, R₂ and R₃ independently or simultaneously represent a (C₁-C₂₀)-alkyl group, a (C₂-C₂₀)-alkenyl group, a (C₂-C₂₀)-alkynyl group, a (C₃-C₂₀)-cycloalkyl group, a —Si[(C₁-C₂₀)-alkyl]₃ group, a (C₆-C₁₄)-aryl group, or a (C₅-C₁₄)-heteroaryl group, or an acyl group —C(═O)—R′, wherein R′ is a (C₁-C₂₀)-alkyl group, wherein each group is each optionally substituted with one or more halogen atoms (F, Cl, Br or I), a —(C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl, and

wherein one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more halogen (F, Cl, Br or I), or a —(C₁-C₂₀)-alkyl groups.

In one embodiment, R₂ and R₃ independently or simultaneously represent a (C₁-C₁₀)-alkyl group, a (C₂-C₁₀)-alkenyl group, a (C₂-C₁₀)-alkynyl group, a (C₃-C₁₀)-cycloalkyl group, a —Si[(C₁-C₁₀)-alkyl]₃ group, a (C₆-C₁₀)-aryl group, or a (C₅-C₁₀)-heteroaryl group, or an acyl group —C(═O)—R′, wherein R′ is a (C₁-C₁₀)-alkyl group, wherein each group is each optionally substituted with one or more halogen atoms (F, Cl, Br or I), a —(C₁-C₁₀)-alkyl group, a (C₂-C₁₀)-alkenyl group, a (C₂-C₁₀)-alkynyl group, —OR^{d}, or —NR^{d}₂, wherein R^{c} and R^{d} are independently or simultaneously hydrogen, (C₁-C₁₀)-alkyl, (C₂-C₁₀)-alkenyl, or (C₂-C₁₀)-alkynyl, and

wherein one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more halogen (F, Cl, Br or I), or a —(C₁-C₁₀)-alkyl groups.

In one embodiment, R₂ and R₃ independently or simultaneously represent a (C₁-C₆)-alkyl group, a (C₂-C₆)-alkenyl group, a (C₂-C₆)-alkynyl group, a (C₃-C₆)-cycloalkyl group, a —Si[(C₁-C₆)-alkyl]₃ group, a phenyl group, or a (C₅-C₆)-heteroaryl group, or an acyl group —C(═O)—R′, wherein R′ is a (C₁-C₆)-alkyl group, wherein each group is each optionally substituted with one or more halogen atoms (F, Cl, Br or I), a —(C₁-C₆)-alkyl group, a (C₂-C₆)-alkenyl group, a (C₂-C₆)-alkynyl group, —OR^{d}, or —NR^{d}₂, wherein R^{c} and R^{d} are independently or simultaneously hydrogen, (C₁-C₆)-alkyl, (C₂-C₆)-alkenyl, or (C₂-C₆)-alkynyl, and

wherein one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more halogen (F, Cl, Br or I), or a —(C₁-C₁₀₆)-alkyl groups.

In one embodiment, R₂ and R₃ independently or simultaneously represent a (C₁-C₆)-alkyl group, a —Si[(C₁-C₆)-alkyl]₃ group, or a phenyl group.

In one embodiment, R₂ and R₃ independently or simultaneously represent a —Si[(C₁-C₆)-alkyl]₃ group. In one embodiment, R₂ and R₃ independently or simultaneously represent a —Si[(C₁-C₃)-alkyl]₃ group. In one embodiment, R₂ and R₃ represent a —Si(CH₃)₃ group.

In one embodiment, the compound of the Formula (II) is a compound of Formula (IIA)

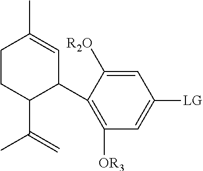

wherein LG is any suitable leaving group. In one embodiment, LG is
(i) an anionic group such as sulphonates, halides or boronates;
(ii) MXₙ groups (M=Li, Mg, Zn, Sn, B, Si; X is halide, OH, OR, (C₁-C₂₀)-alkyl, (C₁-C₂₀)-aryl, etc.; n=0 to 3).

In another embodiment, the boronate leaving group is —B(OR)₂, where R is H, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₄)-aryl group. In another embodiment, the boronate leaving group is —B(OR)₂, where R is H, a (C₁-C₂₀)-alkyl group (such as a (C₁-C₁₀)-alkyl group) or a (C₆-C₁₄)-aryl group (such as a (C₆-C₁₀)-aryl group). In another embodiment, the boronate leaving group is —BF₃K.

In one embodiment, for example, compounds of the Formula (IIA), and subsequently compounds of Formula (II), are prepared as in the following schemes:

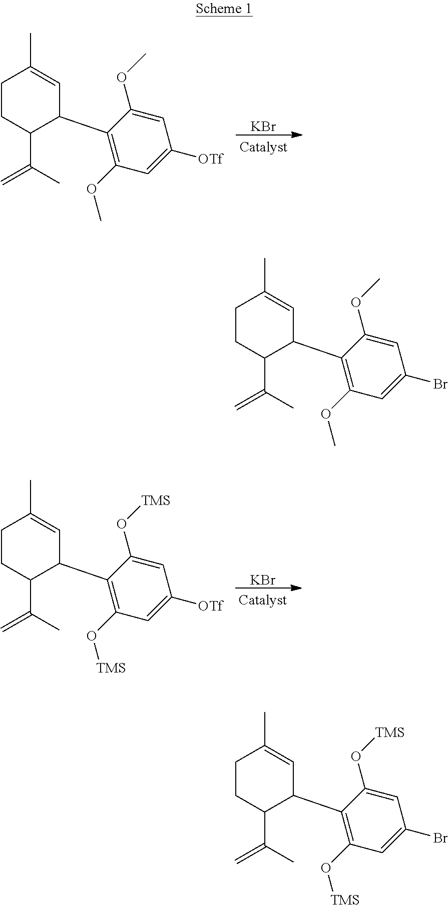

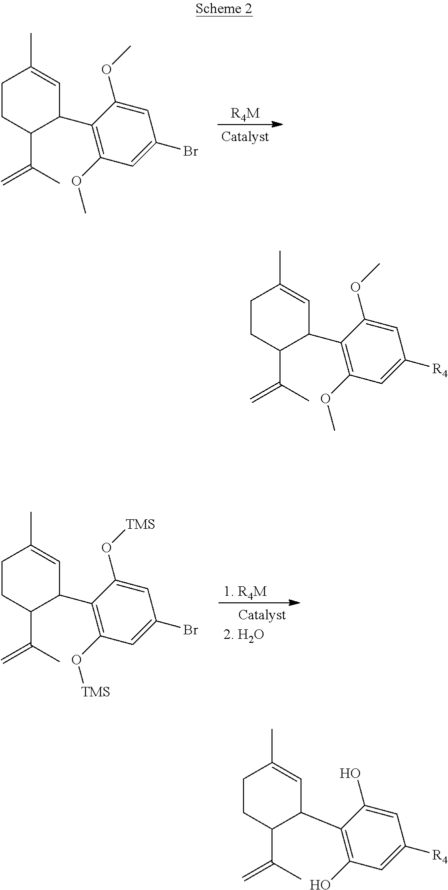

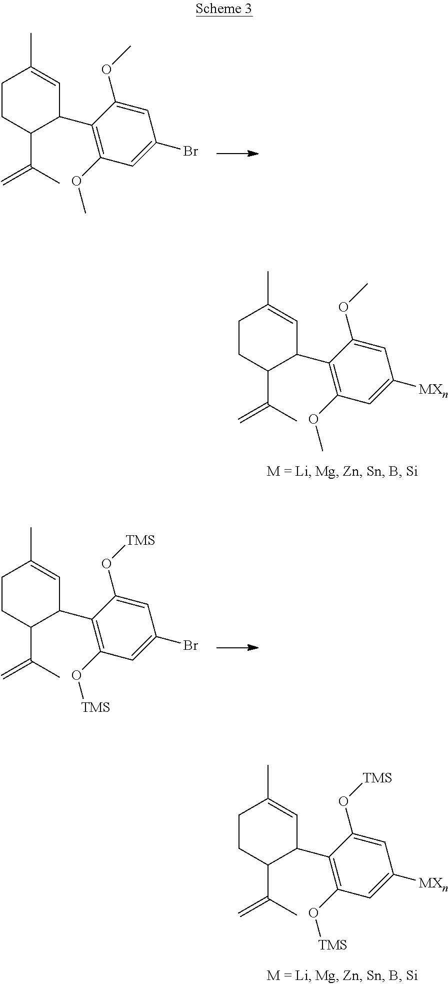

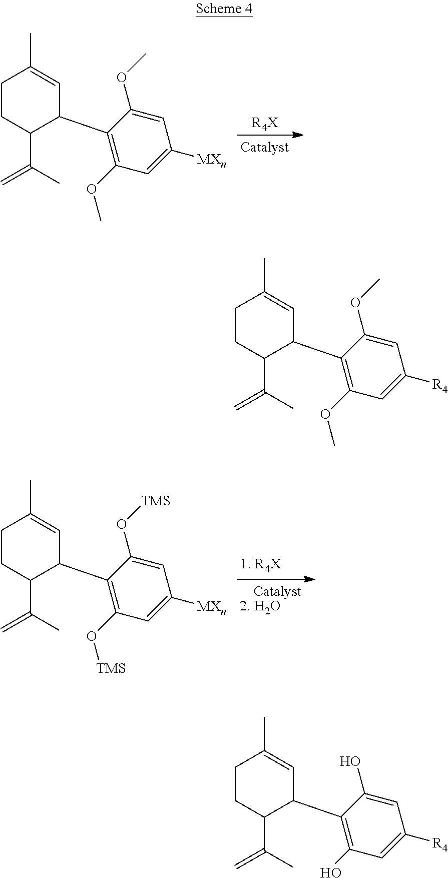

The transformations to which the compounds of the disclosure can be applied include but are not limited to catalytic and non-catalytic carbon-carbon bond forming reactions including Ullman, Suzuki-Miyaura, Negishi, Kumada, Sonogashira and Stille reactions. Such carbon-carbon bond forming reactions include the use of compounds of the disclosure to prepare cannabinoid compounds of Formula (III):

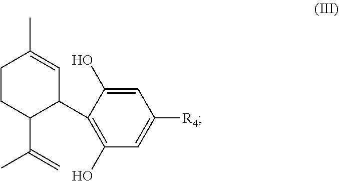

### and Formula (IV):

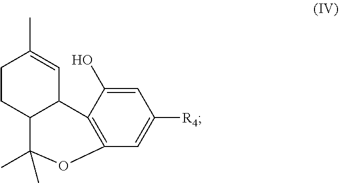

### and Formula (V):

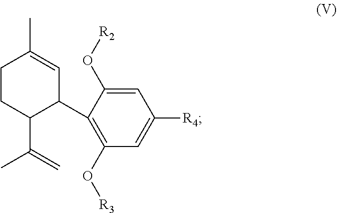

### and Formula (VI):

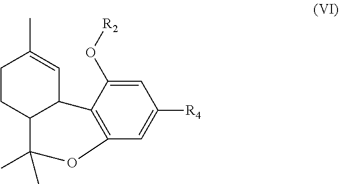

wherein, R₂ and R₃ represents a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more groups; and R₄ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

In one embodiment, R₂ and R₃ in the compounds of Formula (III), (IV) (V) and (VI) are as defined in each embodiment for the compounds of Formula (II).

In one embodiment, R₄ represents a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, wherein the latter 5 groups are each optionally substituted with one or more halogen atoms (F, Cl, Br or I), —(C₁-C₂₀)-alkyl, a (C₂-C₂₀)-alkenyl group, a (C₂-C₂₀)-alkynyl group, (C₆-C₁₄)-aryl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

In one embodiment, R₄ represents a hydrogen atom, a (C₁-C₂₀)-alkyl group, a (C₂-C₂₀)-alkenyl group, a (C₆-C₁₄)-aryl group, wherein the latter 3 groups are each optionally substituted with one or more halogen atoms (F, Cl, Br or I), —(C₁-C₁₀)-alkyl, a (C₂-C₁₀)-alkenyl group, a (C₂-C₁₀)-alkynyl group, or (C₆-C₁₀)-aryl group.

In one embodiment, R₄ represents a hydrogen atom, a (C₁-C₂₀)-alkyl group, a (C₆-C₁₀)-aryl group, wherein the latter 2 groups are each optionally substituted with one or more phenyl groups.

In one embodiment, R₄ represents a hydrogen atom or a (C₁-C₂₀)-alkyl group optionally substituted with a phenyl group.

### (III) Processes of the Disclosure

The present disclosure also relates to a process for the production of compounds of Formula (I) comprising first contacting a compound of Formula (VII)

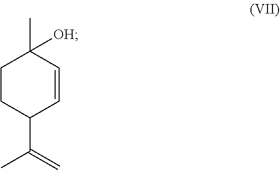

and a compound of Formula (VIII),

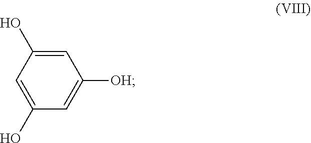

to form a compound of Formula (IX).

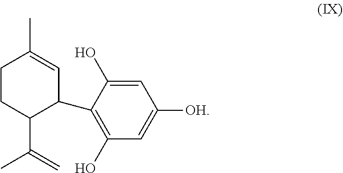 [SMILES: C=C(C)C1CCC(C)=CC1c1c(O)cc(O)cc1O]

Compound (IX) is then transformed to a compound of Formula (I) by contacting a compound of Formula (IX) with the required sulfonylating reagent in the presence of a base.

Compound (I) is then transformed to a compound of Formula (II) by contacting a compound of Formula (I) with a suitable reagent in the presence of a base.

In some aspects, the transformation of Compound (VII) and Compound (VIII) to Compound (IX) requires a suitable acid catalyst. Suitable acid catalysts include but are not limited to Lewis acids, organic acids and inorganic acids.

The disclosure also relates to a process for the catalytic and non-catalytic use of compounds of Formula (I) and Formula (II) to prepare cannabinoid compounds of Formula (III):

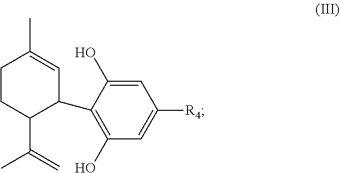

### and Formula (IV):

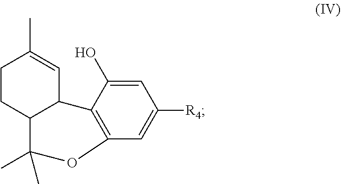

### and Formula (V):

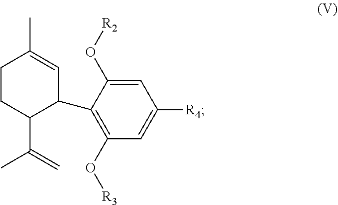

### and Formula (VI):

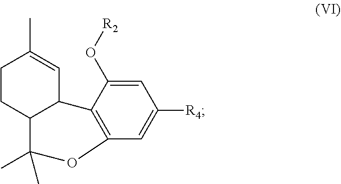

wherein, R₂ and R₃ represents a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more groups; and R₄ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

In one embodiment, R₂, R₃ and R₄ are as defined above.

Carbon-carbon bond forming reactions for the preparation of cannabinoid compounds of Formula (III), Formula (IV), Formula (V) or Formula (VI) include but are not limited to catalytic and non-catalytic Ullman, Suzuki-Miyaura, Negishi, Kumada, Sonogashira and Stille reactions.

In some embodiments of the invention, a compound of Formula (I) or Formula (II) is contacted with a nucleophilic R₄ group, R₄—W wherein R₄ is as defined above and is nucleophilic and W is an electrophilic group, such as a boron containing compound such as R₄—B(OH)₂, R₄—B(OR)₂ or R₄—BF₃K; or a Grignard compound such as R₄—MgX; or an organozinc compound, such as R₄—ZnX, in the presence or absence of a catalyst to produce a compound of Formula (III), Formula (IV), Formula (V) or Formula (VI).

In some embodiments of the invention, the catalytic system characterizing the process of the instant invention may comprise a base. In some embodiments, said base can be any conventional base. In some embodiments, non-limiting examples include: organic non-coordinating bases such as DBU, an alkaline or alkaline-earth metal carbonate, a carboxylate salt such as sodium or potassium acetate, or an alcoholate or hydroxide salt. Preferred bases are the alcoholate or hydroxide salts selected from the group consisting of the compounds of formula (RO)₂M′ and ROM″, wherein M′ is an alkaline-earth metal, M″ is an alkaline metal and R stands for hydrogen or a linear or branched alkyl group.

The catalyst can be added to the reaction medium in a large range of concentrations. As non-limiting examples, one can cite as catalyst concentration values ranging from 0.001% to 50%, relative to the amount of substrate, thus representing respectively a substrate/catalyst (S/cat) ratio of 100,000 to 2. Preferably, the complex concentration will be comprised between 0.01% and 10%, i.e. a S/cat ratio of 10,000 to 10 respectively. In some preferred embodiments, there will be used concentrations in the range of 0.1 to 5%, corresponding to a S/cat ratio of 1000 to 20 respectively.

If required, useful quantities of base, added to the reaction mixture, may be comprised in a relatively large range. In some embodiments, non-limiting examples include: ranges between 1 to 100 molar equivalents relative to the substrate. However, it should be noted that it is also possible to add a small amount of base (e.g. base/substrate=1 to 3) to achieve high yields.

In the processes of this invention, the catalytic reaction can be carried out in the presence or absence of a solvent. When a solvent is required or used for practical reasons, then any solvent currently used in catalytic reactions can be used for the purposes of the invention. Non-limiting examples include aromatic solvents such as benzene, toluene or xylene, hydrocarbon solvents such as hexane or cyclohexane, ethers such as tetrahydrofuran, or yet primary or secondary alcohols, or water, or mixtures thereof. A person skilled in the art is well able to select the solvent most convenient in each case to optimize the catalytic reaction.

The temperature at which the catalytic reaction can be carried out is comprised between −30° C. and 200° C., more preferably in the range of between 0° C. and 100° C. Of course, a person skilled in the art is also able to select the preferred temperature.

Standard catalytic conditions, as used herein, typically implies the mixture of the substrate with the catalyst with or without a base, possibly in the presence of a solvent, and then treating such a mixture with the desired reactant at a chosen temperature in air or under an inert atmosphere of nitrogen or argon gas. Varying the reaction conditions, including for example, catalyst, temperature, solvent and reagent, to optimize the yield of the desired product would be well within the abilities of a person skilled in the art.

The present invention is described in the following Examples, which are set forth to aid in the understanding of the invention, and should not be construed to limit in any way the scope of the invention as defined in the claims which follow thereafter.

### (IV) Compounds of the Formula (X)—Benzyl Cannabidiols

The present disclosure also includes compounds of the Formula (X) which are benzyl cannabidiols having the following structure:

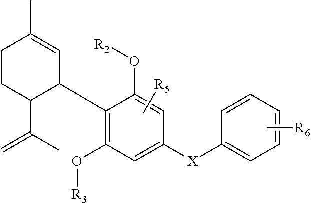

wherein
R₂ and R₃ are as defined above in any paragraph for compounds of the Formula (II);
R₅ and R₆ are one or more substitutents which are hydrogen, halo, —OR^{c}, —NR^{c}₂, carboxylates (—COOR, where R is H or (C₁-C₆)-alkyl), phosphates, sulfates, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, or a (C₅-C₁₄)-heteroaryl group, wherein R^{c} and R^{d} are independently or simultaneously hydrogen, (C₁-C₂₀)-alkyl, (C₂-C₂₀)-alkenyl, or (C₂-C₂₀)-alkynyl;
X is (C₁-C₁₀-alkylene) or (C₂-C₁₀-alkenylene);
and all stereoisomers, and salts thereof.

In one embodiment, R₅ and R₆ are one or more substitutents which are hydrogen, halo, a (C₁-C₁₀)-alkyl group, or a (C₆-C₁₀)-aryl group. In one embodiment, R₅ and R₆ are one or more substitutents which are hydrogen, halo, a (C₁-C₆)-alkyl group, or a phenyl group.

In one embodiment, X is (C₁-C₆-alkylene) or (C₂-C₆-alkenylene). In another embodiment, X is (C₁-C₂-alkylene) or (C₂-alkenylene).

In one embodiment, the compound of the Formula (X) is

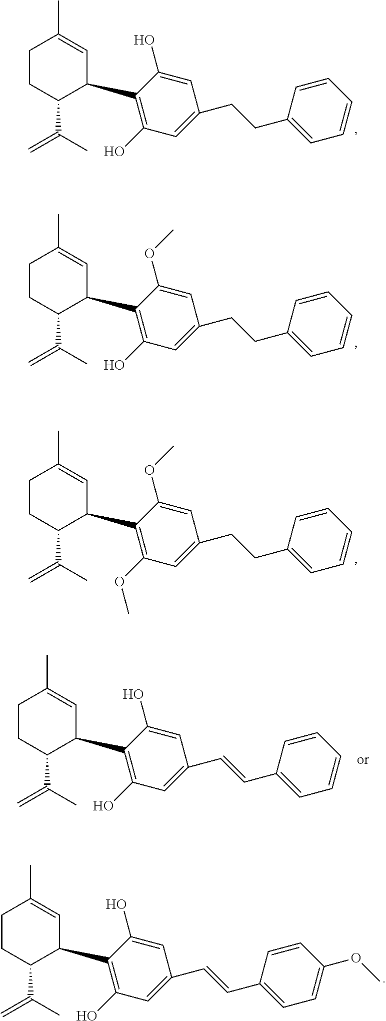 [SMILES: C=C(C)[C@@H]1CCC(C)=C[C@H]1c1c(O)cc(CCc2ccccc2)cc1O ; C=C(C)[C@@H]1CCC(C)=C[C@H]1c1c(O)cc(CCc2ccccc2)cc1OC ; C=C(C)[C@@H]1CCC(C)=C[C@H]1c1c(OC)cc(CCc2ccccc2)cc1OC ; C=C(C)[C@@H]1CCC(C)=C[C@H]1c1c(O)cc(/C=C/c2ccccc2)cc1O ; C=C(C)[C@@H]1CCC(C)=C[C@H]1c1c(O)cc(/C=C/c2ccc(OC)cc2)cc1O]

### EXAMPLES

The disclosure will now be described in further details by way of the following examples, wherein the temperatures are indicated in degrees centigrade and the abbreviations have the usual meaning in the art.

All the procedures described hereafter have been carried out under an inert atmosphere unless stated otherwise. All preparations and manipulations under air-free conditions were carried out under N₂ or Ar atmospheres with the use of standard Schlenk, vacuum line and glove box techniques in dry, oxygen-free solvents. Deuterated solvents were degassed and dried over activated molecular sieves. NMR spectra were recorded on a 300 MHz spectrometer (300 MHz for ¹H, 75 MHz for ¹³C and 121.5 MHz for ³¹P) or a 400 MHz spectrometer (400 MHz for ¹H, 100 MHz for ¹³C and 162 MHz for ³¹P). All ³¹P chemical shifts were measured relative to 85% H₃PO₄ as an external reference. ¹H and ¹³C chemical shifts were measured relative to partially deuterated solvent peaks but are reported relative to tetramethylsilane.

### Example 1. Preparation of 2-((1R,6R)-3-methyl-6-(prop-1-en-2-yl)cyclohex-2-enyl)benzene-1,3,5-triol

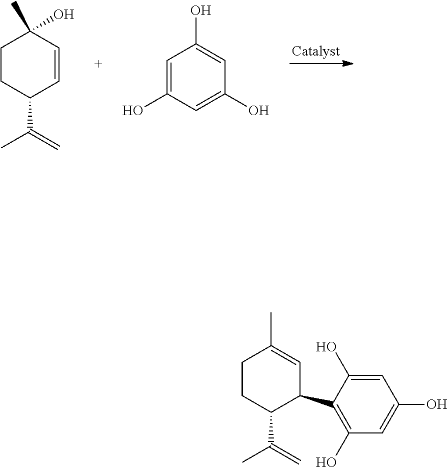 [SMILES: C=C(C)[C@H]1C=C[C@@](C)(O)CC1 ; Oc1cc(O)cc(O)c1 ; C=C(C)[C@@H]1CCC(C)=C[C@H]1c1c(O)cc(O)cc1O]

Anhydrous ethanol (400 ml) and dichloromethane (800 ml) were added to a mixture of 1,3,5-trihydroxybenzene (91.1 g, 722 mmol) and anhydrous magnesium sulfate (100 g, 834 mmol) and the suspension was cooled to 0° C. Tetrafluoroboric acid diethyl ether (7.0 g, 43 mmol) was added slowly with stirring. A solution of (1S,4R)-1-methyl-4-(prop-1-en-2-yl)cyclohex-2-enol (100.0 g, 656 mmol) in dichloromethane (800 ml) was added slowly over 2 hours and 45 minutes at 0° C. with stirring. The mixture was allowed to warm to room temperature and stirred for 1.5 hours. The reaction mixture was filtered and the residue was washed with dichloromethane. The filtrate was washed with water (600 ml) containing NaHCO3 (15 grams). The aqueous portion was extracted with dichloromethane and the combined organic layers were washed with brine (300 ml) then dried (MgSO4). It was filtered and the solvent was removed under reduced pressure to give a viscous, sticky residue. Yield of the crude product=168 grams.

Dichloromethane (470 ml) was added to the crude product and the mixture was stirred for 2 hours. It was filtered and the white crystalline solids were washed with dichloromethane (3×135 ml). The solids were dried under vacuum to give a first crop of product. Yield=70.30 g.

The mother liquor and washings were combined and the solvents were removed and the residue dried under vacuum. Dichloromethane (240 ml) was added and the mixture stirred for 90 minutes. It was filtered and the white crystalline solids were washed with dichloromethane (3×35 ml). The solids were dried under vacuum to give a second crop of product. Yield=18.0 g.

The mother liquor and washings were combined and the solvents were removed and the residue dried under vacuum. Dichloromethane (125 ml) was added and the mixture stirred for 2 hours. It was filtered and the white crystalline solids were washed with dichloromethane (3×12 ml). The solids were dried under vacuum to give a third crop of product. Yield=5.2 g.

Total yield=93.5 g.

### Example 2. Preparation of 3,5-dihydroxy-4-((1R,6R)-3-methyl-6-(prop-1-en-2-yl)cyclohex-2-enyl)phenyl trifluoromethanesulfonate

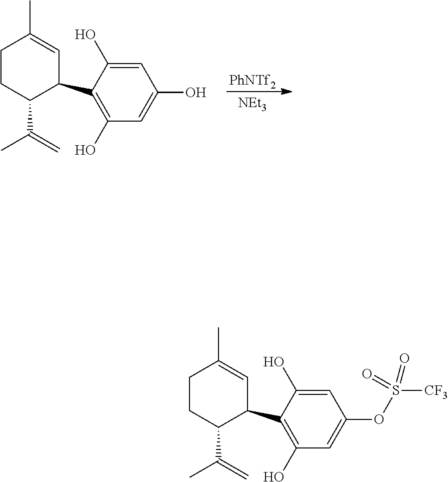 [SMILES: "PhNTf2" ; C=C(C)[C@@H]1CCC(C)=C[C@H]1c1c(O)cc(O)cc1O ; C=C(C)[C@@H]1CCC(C)=C[C@H]1c1c(O)cc(OS(=O)(=O)C(F)(F)F)cc1O ; "NEt3"]

Triethylamine (108.3 g, 1.07 mole) was added to a mixture of 2-((1R,6R)-3-methyl-6-(prop-1-en-2-yl)cyclohex-2-enyl)benzene-1,3,5-triol (93.5 g, 308.8 mmol) in dichloromethane (900 ml) at room temperature while stirring. Solid N-Phenyl-bis(trifluoromethanesulfonimide) (118.61 g, 332 mmol) was added over 1.5 hours and the mixture was kept at room temperature using a water bath. The mixture was stirred at room temperature overnight then quenched with water (350 ml) and the phases separated. The aqueous layer was extracted with dichloromethane (3×100 ml) and the combined organic layers were dried (MgSO₄). It was filtered through a short pad of silica gel and the solvent was removed under reduced pressure. The residue was dissolved in hexanes/CH₂Cl₂ (100 ml of a 1:3 mixture) and filtered through a short silica gel pad, and eluted with hexanes/CH₂Cl₂ (1:3) until no product was detected from the eluent (TLC). The filtrate was evaporated to give the crude product. Yield of crude product=118 g.

Hexanes (120 ml) was added to the crude product and the mixture was stirred for 2 hours. It was filtered and the white, crystalline solids were washed with hexanes and dried under vacuum to give a first crop of product. Yield=72.5 g.

The mother liquor and washings were combined and evaporated to dryness. It was dissolved in EA/hexanes (70 ml of a 3:4 mixture) and filtered through a short silica gel pad, and eluted with EA/hexanes (1:5). The filtrate was evaporated to dryness and hexanes (40 ml) added to the residue (36 g) and the mixture stirred for 1 hour. It was filtered and the white, crystalline solids were washed with hexanes and dried under vacuum to give a second crop of product.

Yield=25.0 g.

The remaining residue was chromatographed using hexanes/EA (6:1) to give a third crop of product. Yield=7.0 grams.

Total yield=104.5 g.

### Example 3. Preparation of 4-((1R,6R)-3-methyl-6-(prop-1-en-2-yl)cyclohex-2-enyl)-3,5-bis(trimethylsilyloxy)phenyl Trifluoromethanesulfonate

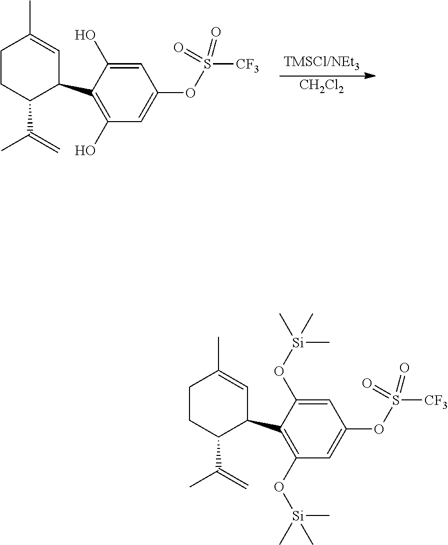

TMSCl (144 g, 1.32 mole) was added to a mixture of 3,5-dihydroxy-4-((1R,6R)-3-methyl-6-(prop-1-en-2-yl)cyclohex-2-enyl)phenyl trifluoromethanesulfonate (104 g, 265 mmol) and NEt₃ (134 g, 1.33 mole) in CH₂Cl₂ (600 ml) at 0° C. The mixture was stirred overnight at room temperature. It was filtered and the solids were washed with dichloromethane. The volatiles were removed from the combined filtrate under reduced pressure. The residue was suspended in hexanes (800 ml) and stirred for 2 hours at room temperature. The mixture was filtered and the solvent was removed under reduced pressure, and the residue was dried under vacuum to give the product as a pale yellow oil. Yield=135 g.

### Example 4. Preparation of 2-((1R,6R)-3-methyl-6-(prop-1-en-2-yl)cyclohex-2-enyl)-5-pentylbenzene-1,3-diol (cannabidiol)

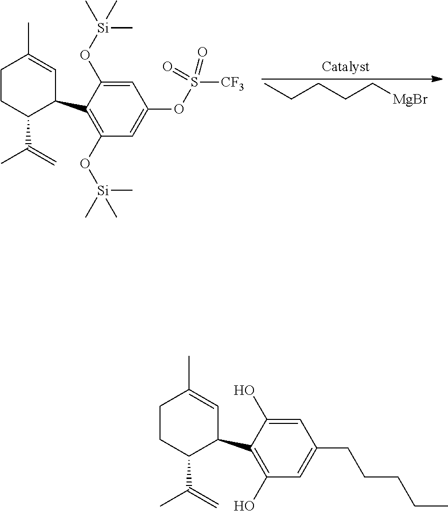

A solution of n-pentylmagnesium bromide (14 ml of a 2.0 M solution in diethyl ether, 28 mmol) was added to a mixture of ZnBr₂ (6.3 g, 28 mmol) and LiBr (3.0 g, 34 mmol) in THF (40 ml) and the suspension was stirred for 30 minutes under argon. A solution of 4-((1R,6R)-3-methyl-6-(prop-1-en-2-yl)cyclohex-2-enyl)-3,5-bis(trimethylsilyloxy)phenyl trifluoromethanesulfonate (10.0 g, 18.6 mmol) and PdCl₂(dppf) (140 mg, 0.19 mmol) in THF (40 ml) was added and the mixture was stirred at room temperature for 2 hours under argon. Water (20 ml) was added followed by 2M H₂SO₄ (10 ml) and the mixture stirred at room temperature for 1 hour. The phases were separated and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale yellow oil which crystallized on standing at room temperature. Yield=5.25 g.

### Example 5. Preparation of (6aR,10aR)-6,6,9-trimethyl-3-pentyl-6a,7,8,10a-tetrahydro-6H-benzo[c]chromen-1-ol (Tetrahydrocannabinol)

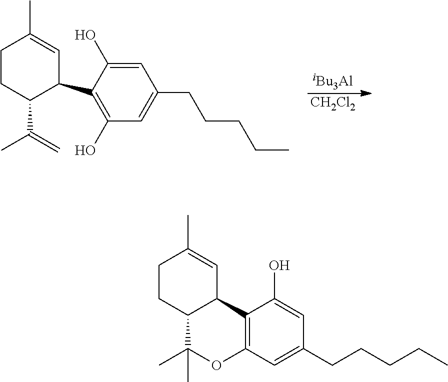 [SMILES: "iBu3Al" ; C=C(C)[C@@H]1CCC(C)=C[C@H]1c1c(O)cc(CCCCC)cc1O ; "CH2Cl2" ; CCCCCc1cc(O)c2c(c1)OC(C)(C)[C@@H]1CCC(C)=C[C@@H]21]

A solution of triisobutylaluminum (0.6 ml of a 1.0 M solution in hexanes, 0.6 mmol) was added to a solution of 2-((1R,6R)-3-methyl-6-(prop-1-en-2-yl)cyclohex-2-enyl)-5-pentylbenzene-1,3-diol (2.0 g, 6.36 mmol) in dichloromethane (35 ml) and the mixture was stirred at room temperature for 24 hours. The reaction was quenched with ammonium chloride solution and diethyl ether was added. The phases were separated and the organic layer was dried (MgSO₄), filtered and evaporated to dryness to give the product as a pale yellow resin. Yield=1.65 g.

### Example 6. Preparation of 5-methyl-2-((1R,6R)-3-methyl-6-(prop-1-en-2-yl)cyclohex-2-enyl)benzene-1,3-diol (Cannabidiorcol)

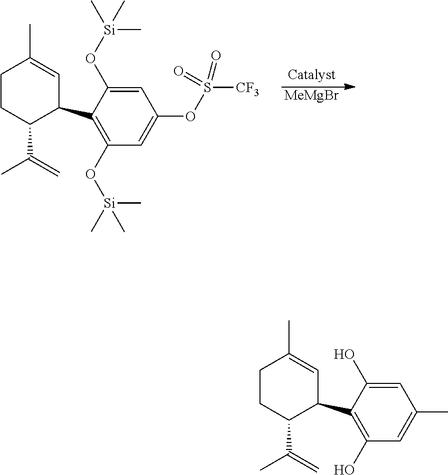 [SMILES: "MeMgBr" ; C=C(C)[C@@H]1CCC(C)=C[C@H]1c1c(O[Si](C)(C)C)cc(OS(=O)(=O)C(F)(F)F)cc1O[Si](C)(C)C ; C=C(C)[C@@H]1CCC(C)=C[C@H]1c1c(O)cc(C)cc1O]

A solution of methylmagnesium bromide (1.4 ml of a 2.0 M solution in diethyl ether, 2.8 mmol) was added to a mixture of ZnBr₂ (0.63 g, 2.8 mmol) and LiBr (0.3 g, 3.4 mmol) in THF (4 ml) and the suspension was stirred for 30 minutes under argon. A solution of 4-((1R,6R)-3-methyl-6-(prop-1-en-2-yl)cyclohex-2-enyl)-3,5-bis(trimethylsilyloxy)phenyl trifluoromethanesulfonate (1.0 g, 1.86 mmol) and PdCl₂(dppf) (14 mg, 0.019 mmol) in THF (4 ml) was added and the mixture was stirred at 40° C. for 24 hours under argon. Water (2 ml) was added followed by 2M H₂SO₄ (1.0 ml) and the mixture stirred at room temperature for 1 hour. The phases were separated and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale yellow oil. Yield=0.46 g.

### Example 7. Preparation of 5-ethyl-2-((1R,6R)-3-methyl-6-(prop-1-en-2-yl)cyclohex-2-enyl)benzene-1,3-diol

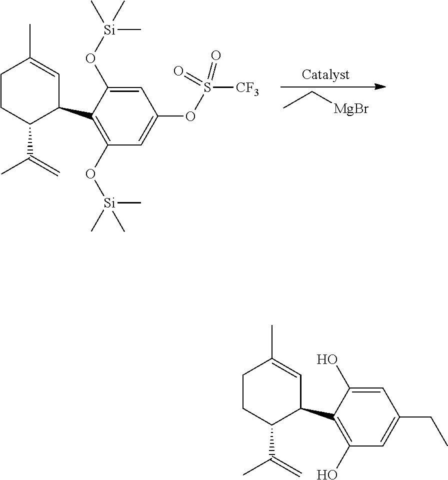

A solution of ethylmagnesium bromide (1.4 ml of a 2.0 M solution in diethyl ether, 2.8 mmol) was added to a mixture of ZnBr₂ (0.63 g, 2.8 mmol) and LiBr (0.3 g, 3.4 mmol) in THF (4 ml) and the suspension was stirred for 30 minutes under argon. A solution of 4-((1R,6R)-3-methyl-6-(prop-1-en-2-yl)cyclohex-2-enyl)-3,5-bis(trimethylsilyloxy)phenyl trifluoromethanesulfonate (1.0 g, 1.86 mmol) and PdCl₂(dppf) (14 mg, 0.019 mmol) in THF (4 ml) was added and the mixture was stirred at room temperature for 12 hours under argon. Water (2 ml) was added followed by 2M H₂SO₄ (1.0 ml) and the mixture stirred at room temperature for 1 hour. The phases were separated and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale yellow oil. Yield=0.48 g.

### Example 8. Preparation of 2-((1R,6R)-3-methyl-6-(prop-1-en-2-yl)cyclohex-2-enyl)-5-propylbenzene-1,3-diol (Cannabidivarin)

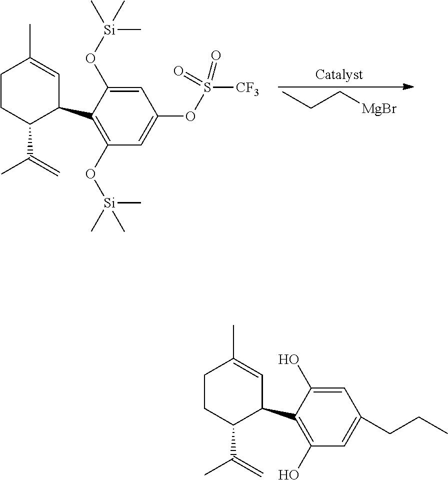

A solution of propylmagnesium bromide (1.4 ml of a 2.0 M solution in diethyl ether, 2.8 mmol) was added to a mixture of ZnBr₂ (0.63 g, 2.8 mmol) and LiBr (0.3 g, 3.4 mmol) in THF (4 ml) and the suspension was stirred for 30 minutes under argon. A solution of 4-((1R,6R)-3-methyl-6-(prop-1-en-2-yl)cyclohex-2-enyl)-3,5-bis(trimethylsilyloxy)phenyl trifluoromethanesulfonate (1.0 g, 1.86 mmol) and PdCl₂(dppf) (14 mg, 0.019 mmol) in THF (4 ml) was added and the mixture was stirred at room temperature for 6 hours under argon. Water (2 ml) was added followed by 2M H₂SO₄ (1.0 ml) and the mixture stirred at room temperature for 1 hour. The phases were separated and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale yellow oil. Yield=0.52 g.

### Example 9. Preparation of 5-butyl-2-((1R,6R)-3-methyl-6-(prop-1-en-2-yl)cyclohex-2-enyl)benzene-1,3-diol (Cannabidibutol)

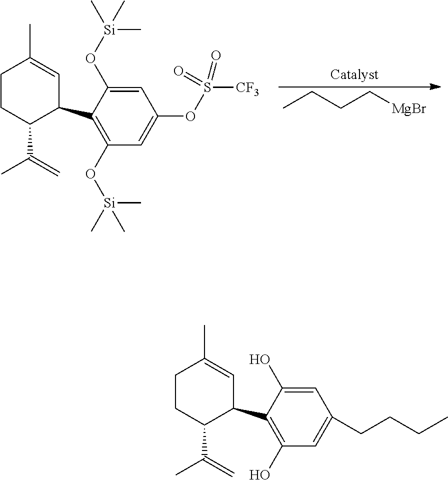

A solution of butylmagnesium bromide (1.4 ml of a 2.0 M solution in diethyl ether, 2.8 mmol) was added to a mixture of ZnBr₂ (0.63 g, 2.8 mmol) and LiBr (0.3 g, 3.4 mmol) in THF (4 ml) and the suspension was stirred for 30 minutes under argon. A solution of 4-((1R,6R)-3-methyl-6-(prop-1-en-2-yl)cyclohex-2-enyl)-3,5-bis(trimethylsilyloxy)phenyl trifluoromethanesulfonate (1.0 g, 1.86 mmol) and PdCl₂(dppf) (14 mg, 0.019 mmol) in THF (4 ml) was added and the mixture was stirred at room temperature for 12 hours under argon. Water (2 ml) was added followed by 2M H₂SO₄ (1.0 ml) and the mixture stirred at room temperature for 1 hour. The phases were separated and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale yellow oil. Yield=0.54 g.

### Example 10. Preparation of 5-hexyl-2-((1R,6R)-3-methyl-6-(prop-1-en-2-yl)cyclohex-2-enyl)benzene-1,3-diol

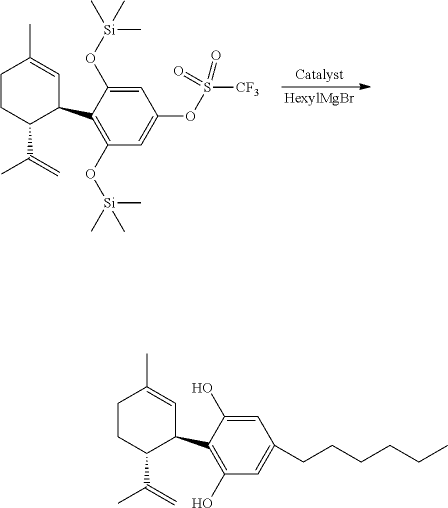 [SMILES: C=C(C)[C@@H]1CCC(C)=C[C@H]1c1c(O[Si](C)(C)C)cc(OS(=O)(=O)C(F)(F)F)cc1O[Si](C)(C)C ; C=C(C)[C@@H]1CCC(C)=C[C@H]1c1c(O)cc(CCCCCC)cc1O]

This was prepared according to the procedure outlined in Example 9 and using hexylmagnesium bromide. The product was isolated as a pale yellow oil. Yield=0.59 g.

### Example 11. Preparation of 5-heptyl-2-((1R,6R)-3-methyl-6-(prop-1-en-2-yl)cyclohex-2-enyl)benzene-1,3-diol (Cannabidiphorol)

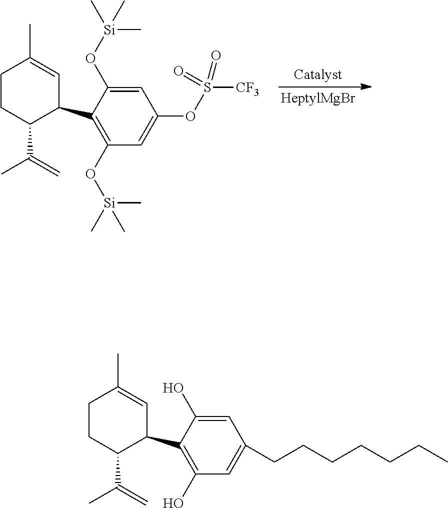 [SMILES: C=C(C)[C@@H]1CCC(C)=C[C@H]1c1c(O[Si](C)(C)C)cc(OS(=O)(=O)C(F)(F)F)cc1O[Si](C)(C)C ; C=C(C)[C@@H]1CCC(C)=C[C@H]1c1c(O)cc(CCCCCCC)cc1O]

This was prepared according to the procedure outlined in Example 9 and using heptylmagnesium bromide. The product was isolated as a pale yellow oil. Yield=0.62 g.

### Example 12. Preparation of 2-((1R,6R)-3-methyl-6-(prop-1-en-2-yl)cyclohex-2-enyl)-5-octylbenzene-1,3-diol

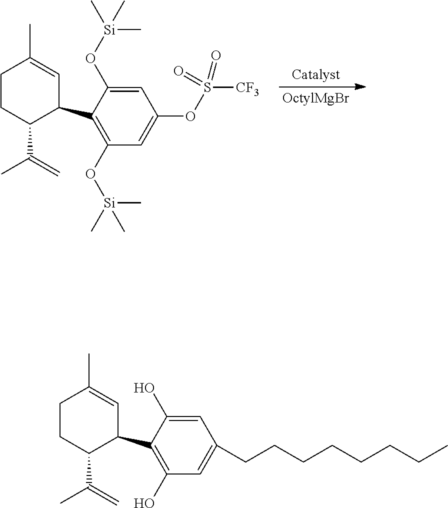 [SMILES: C=C(C)[C@@H]1CCC(C)=C[C@H]1c1c(O[Si](C)(C)C)cc(OS(=O)(=O)C(F)(F)F)cc1O[Si](C)(C)C ; C=C(C)[C@@H]1CCC(C)=C[C@H]1c1c(O)cc(CCCCCCCC)cc1O]

This was prepared according to the procedure outlined in Example 9 and using octylmagnesium bromide. The product was isolated as a pale yellow oil. Yield=0.65 g.

### Example 13. Preparation of 2-((1R,6R)-3-methyl-6-(prop-1-en-2-yl)cyclohex-2-enyl)-5-nonylbenzene-1,3-diol

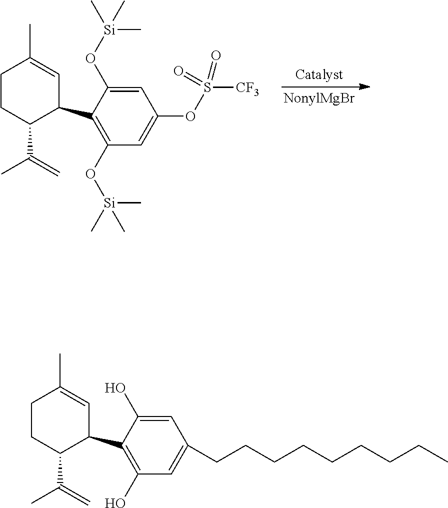 [SMILES: C=C(C)[C@@H]1CCC(C)=C[C@H]1c1c(O[Si](C)(C)C)cc(OS(=O)(=O)C(F)(F)F)cc1O[Si](C)(C)C ; C=C(C)[C@@H]1CCC(C)=C[C@H]1c1c(O)cc(CCCCCCCCC)cc1O]

This was prepared according to the procedure outlined in Example 9 and using nonylmagnesium bromide. The product was isolated as a pale yellow oil. Yield=0.68 g.

### Example 14. Preparation of 5-decyl-2-((1R,6R)-3-methyl-6-(prop-1-en-2-yl)cyclohex-2-enyl)benzene-1,3-diol

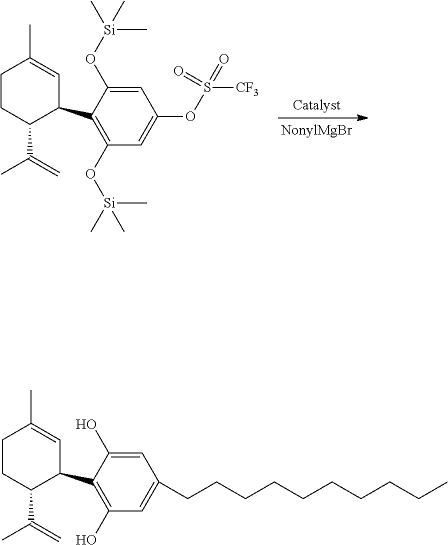 [SMILES: C=C(C)[C@@H]1CCC(C)=C[C@H]1c1c(O[Si](C)(C)C)cc(OS(=O)(=O)C(F)(F)F)cc1O[Si](C)(C)C ; C=C(C)[C@@H]1CCC(C)=C[C@H]1c1c(O)cc(CCCCCCCCCC)cc1O]

This was prepared according to the procedure outlined in Example 9 and using dioctylmagnesium bromide. The product was isolated as a pale yellow oil. Yield=0.70 g.

### Example 15. Preparation of 5-icosyl-2-((1R,6R)-3-methyl-6-(prop-1-en-2-yl)cyclohex-2-enyl)benzene-1,3-diol

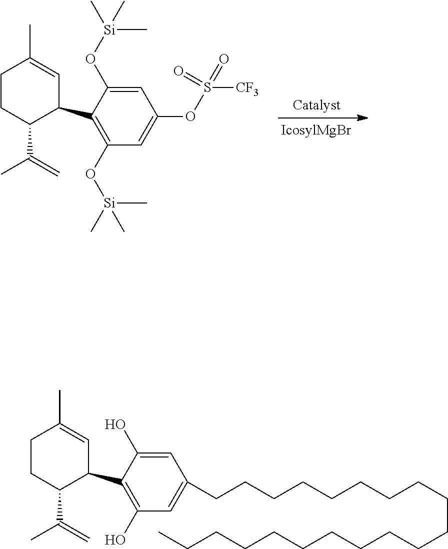 [SMILES: C=C(C)[C@@H]1CCC(C)=C[C@H]1c1c(O[Si](C)(C)C)cc(OS(=O)(=O)C(F)(F)F)cc1O[Si](C)(C)C ; C=C(C)[C@@H]1CCC(C)=C[C@H]1c1c(O)cc(CCCCCCCCCCCCCCCCCCCC)cc1O]

This was prepared according to the procedure outlined in Example 9 and using icosylmagnesium bromide. The product was isolated as a white solid. Yield=0.95 g.

### Example 16. Preparation of 2-((1R,6R)-3-methyl-6-(prop-1-en-2-yl)cyclohex-2-enyl)-5-phenethylbenzene-1,3-diol

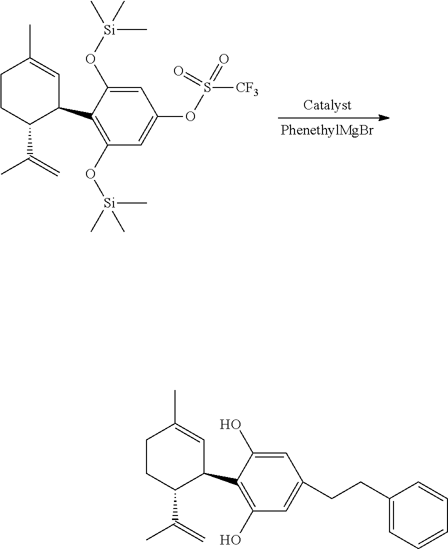 [SMILES: C=C(C)[C@@H]1CCC(C)=C[C@H]1c1c(O[Si](C)(C)C)cc(OS(=O)(=O)C(F)(F)F)cc1O[Si](C)(C)C ; C=C(C)[C@@H]1CCC(C)=C[C@H]1c1c(O)cc(CCc2ccccc2)cc1O]

A solution of phenethylmagnesium bromide (1.4 ml of a 2.0 M solution in diethyl ether, 2.8 mmol) was added to a mixture of ZnBr₂ (0.63 g, 2.8 mmol) and LiBr (0.3 g, 3.4 mmol) in THF (4 ml) and the suspension was stirred for 30 minutes under argon. A solution of 4-((1R,6R)-3-methyl-6-(prop-1-en-2-yl)cyclohex-2-enyl)-3,5-bis(trimethylsilyloxy)phenyl trifluoromethanesulfonate (1.0 g, 1.86 mmol) and PdCl₂(dppf) (14 mg, 0.019 mmol) in THF (4 ml) was added and the mixture was stirred at 50° C. for 24 hours under argon. Water (2 ml) was added followed by 2M H₂SO₄ (1.0 ml) and the mixture stirred at room temperature for 1 hour. The phases were separated and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale yellow oil which was purified by chromatography. Yield=0.61 g.

### Example 17. Preparation of 2-((1R,6R)-3-methyl-6-(prop-1-en-2-yl)cyclohex-2-enyl)-5-styrylbenzene-1,3-diol

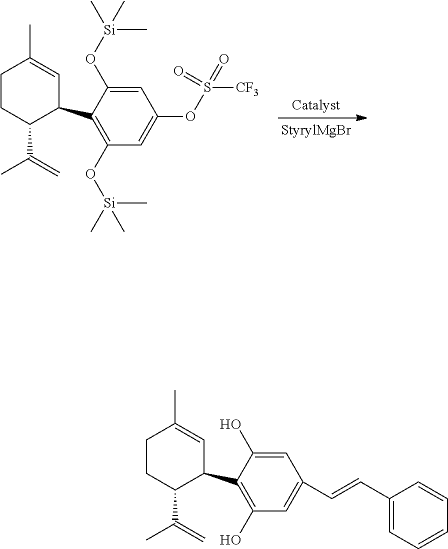 [SMILES: C=C(C)[C@@H]1CCC(C)=C[C@H]1c1c(O[Si](C)(C)C)cc(OS(=O)(=O)C(F)(F)F)cc1O[Si](C)(C)C ; C=C(C)[C@@H]1CCC(C)=C[C@H]1c1c(O)cc(/C=C/c2ccccc2)cc1O]

This was prepared according to the procedure outlined in Example 16 and using styrylmagnesium bromide. The product was isolated as a pale yellow oil. Yield=0.58 g.

### Example 18. Preparation of 5-(4-methoxystyryl)-2-((1R,6R)-3-methyl-6-(prop-1-en-2-yl)cyclohex-2-enyl)benzene-1,3-diol

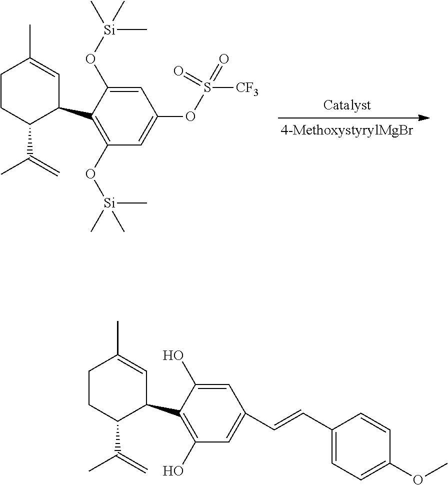 [SMILES: C=C(C)[C@@H]1CCC(C)=C[C@H]1c1c(O[Si](C)(C)C)cc(OS(=O)(=O)C(F)(F)F)cc1O[Si](C)(C)C ; C=C(C)[C@@H]1CCC(C)=C[C@H]1c1c(O)cc(/C=C/c2ccc(OC)cc2)cc1O]

This was prepared according to the procedure outlined in Example 16 and using 4-methoxystyrylmagnesium bromide. The product was isolated as yellow oil which was purified by chromatography. Yield=0.63 g.

### Example 19. Preparation of (6aR,10aR)-3,6,6,9-tetramethyl-6a,7,8,10a-tetrahydro-6H-benzo[c]chromen-1-ol (Tetrahydrocannabiorcol)

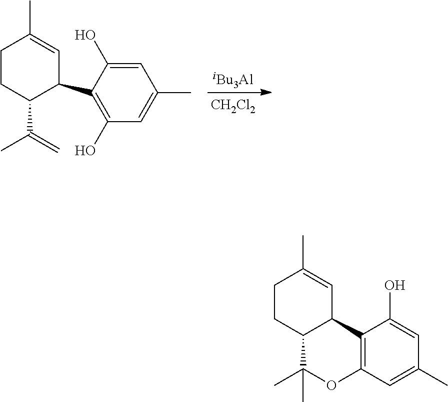

A solution of triisobutylaluminum (0.15 ml of a 1.0 M solution in hexanes, 0.15 mmol) was added to a solution of 5-methyl-2-((1R,6R)-3-methyl-6-(prop-1-en-2-yl)cyclohex-2-enyl)benzene-1,3-diol (413 mg, 1.6 mmol) in dichloromethane (10 ml) and the mixture was stirred at room temperature for 24 hours. The reaction was quenched with ammonium chloride solution and diethyl ether was added. The phases were separated and the organic layer was dried (MgSO₄), filtered and evaporated to dryness to give the product as a pale yellow resin. Yield=340 mg.

### Example 20. Preparation of (6aR,10aR)-3-ethyl-6,6,9-trimethyl-6a,7,8,10a-tetrahydro-6H-benzo[c]chromen-1-ol

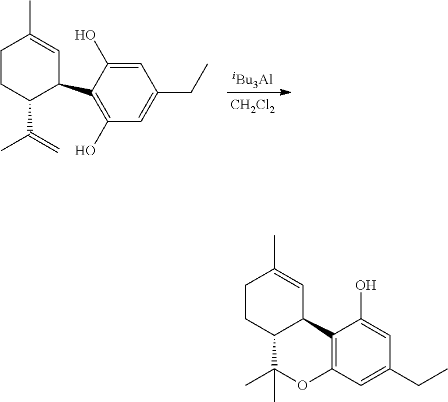

This was prepared according to the procedure outlined in Example 19 and using 5-ethyl-2-((1R,6R)-3-methyl-6-(prop-1-en-2-yl)cyclohex-2-enyl)benzene-1,3-diol (436 mg, 1.6 mmol). The product was isolated as a pale yellow oil. Yield=346 mg.

### Example 21. Preparation of (6aR,10aR)-6,6,9-trimethyl-3-propyl-6a,7,8,10a-tetrahydro-6H-benzo[c]chromen-1-ol (Tetrahydrocannabivarin)

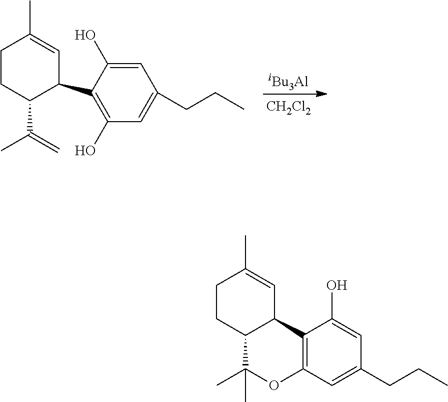

This was prepared according to the procedure outlined in Example 19 and using 2-((1R,6R)-3-methyl-6-(prop-1-en-2-yl)cyclohex-2-enyl)-5-propylbenzene-1,3-diol (458 mg, 1.6 mmol). The product was isolated as a pale yellow oil. Yield=362 mg.

### Example 22. Preparation of (6aR,10aR)-3-butyl-6,6,9-trimethyl-6a,7,8,10a-tetrahydro-6H-benzo[c]chromen-1-ol (Tetrahydrocannabutol)

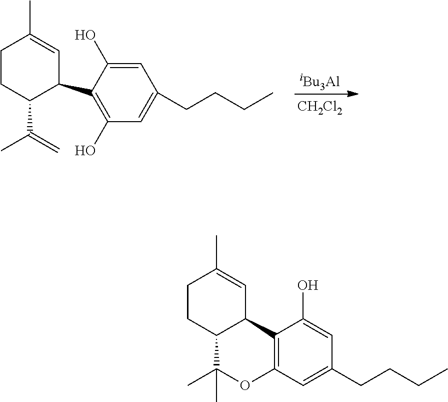

This was prepared according to the procedure outlined in Example 19 and using 5-butyl-2-((1R,6R)-3-methyl-6-(prop-1-en-2-yl)cyclohex-2-enyl)benzene-1,3-diol (481 mg, 1.6 mmol). The product was isolated as a pale yellow oil. Yield=367 mg.

### Example 23. Preparation of (6aR,10aR)-3-hexyl-6,6,9-trimethyl-6a,7,8,10a-tetrahydro-6H-benzo[c]chromen-1-ol

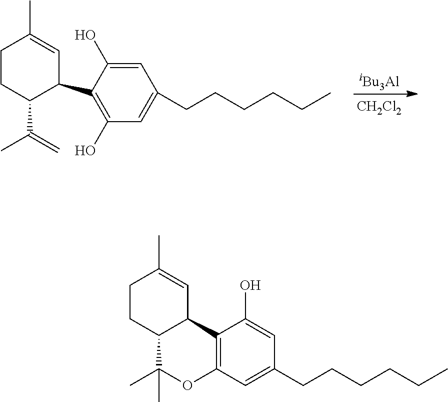

This was prepared according to the procedure outlined in Example 19 and using 5-hexyl-2-((1R,6R)-3-methyl-6-(prop-1-en-2-yl)cyclohex-2-enyl)benzene-1,3-diol (526 mg, 1.6 mmol). The product was isolated as a pale yellow resin. Yield=452 mg.

### Example 24. Preparation of (6aR,10aR)-3-heptyl-6,6,9-trimethyl-6a,7,8,10a-tetrahydro-6H-benzo[c]chromen-1-ol (Tetrahydrocannabiphorol)

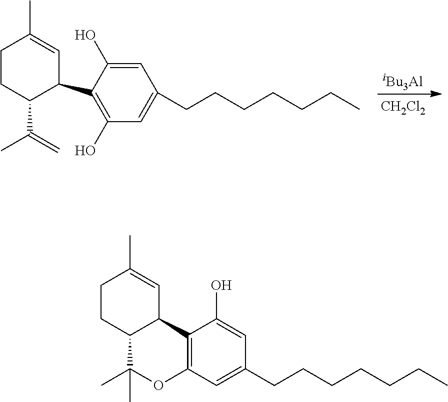

This was prepared according to the procedure outlined in Example 19 and using 5-heptyl-2-((1R,6R)-3-methyl-6-(prop-1-en-2-yl)cyclohex-2-enyl)benzene-1,3-diol (548 mg, 1.6 mmol). The product was isolated as a pale yellow resin. Yield=475 mg.

### Example 25. Preparation of (6aR,10aR)-6,6,9-trimethyl-3-octyl-6a,7,8,10a-tetrahydro-6H-benzo[c]chromen-1-ol

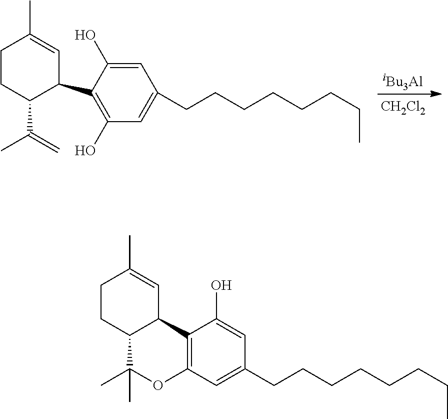

This was prepared according to the procedure outlined in Example 19 and using 2-((1R,6R)-3-methyl-6-(prop-1-en-2-yl)cyclohex-2-enyl)-5-octylbenzene-1,3-diol (570 mg, 1.6 mmol). The product was isolated as a pale yellow resin. Yield=494 mg.

### Example 26. Preparation of (6aR,10aR)-6,6,9-trimethyl-3-nonyl-6a,7,8,10a-tetrahydro-6H-benzo[c]chromen-1-ol

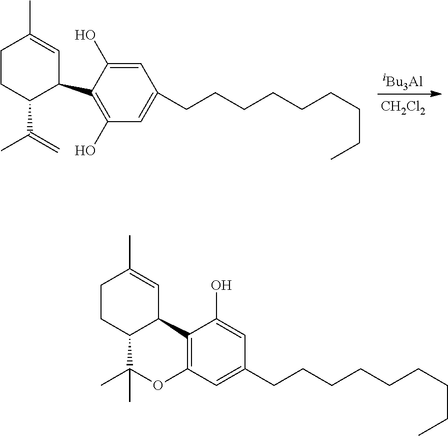

This was prepared according to the procedure outlined in Example 19 and using 2-((1R,6R)-3-methyl-6-(prop-1-en-2-yl)cyclohex-2-enyl)-5-nonylbenzene-1,3-diol (593 mg, 1.6 mmol). The product was isolated as a pale yellow resin. Yield=532 mg.

### Example 27. Preparation of (6aR,10aR)-3-decyl-6,6,9-trimethyl-6a,7,8,10a-tetrahydro-6H-benzo[c]chromen-1-ol

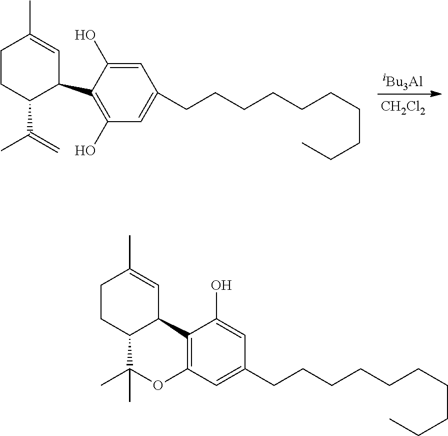

This was prepared according to the procedure outlined in Example 19 and using 5-decyl-2-((1R,6R)-3-methyl-6-(prop-1-en-2-yl)cyclohex-2-enyl)benzene-1,3-diol (615 mg, 1.6 mmol). The product was isolated as a pale yellow resin. Yield=565 mg.

### Example 28. Preparation of (6aR,10aR)-3-icosyl-6,6,9-trimethyl-6a,7,8,10a-tetrahydro-6H-benzo[c]chromen-1-ol

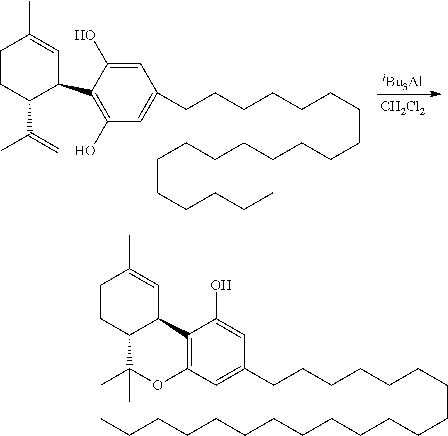

This was prepared according to the procedure outlined in Example 19 and using 5-icosyl-2-((1R,6R)-3-methyl-6-(prop-1-en-2-yl)cyclohex-2-enyl)benzene-1,3-diol (840 mg, 1.6 mmol). The product was isolated as a white solid. Yield=802 mg.

### Example 29. Preparation of (6aR,10aR)-6,6,9-trimethyl-3-phenethyl-6a,7,8,10a-tetrahydro-6H-benzo[c]chromen-1-ol

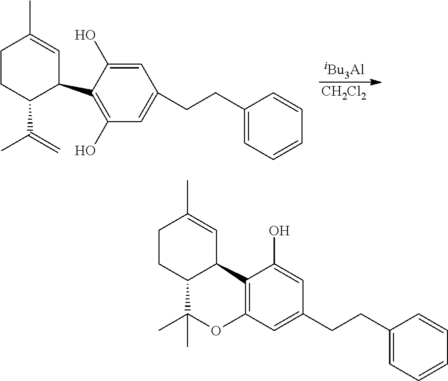

This was prepared according to the procedure outlined in Example 19 and using 2-((1R,6R)-3-methyl-6-(prop-1-en-2-yl)cyclohex-2-enyl)-5-phenethylbenzene-1,3-diol (558 mg, 1.6 mmol). The product was isolated as pale yellow resin. Yield=492 mg.

### Example 30. Preparation of 2-((1S,6R)-3-methyl-6-(prop-1-en-2-yl)cyclohex-2-enyl)benzene-1,3,5-triol

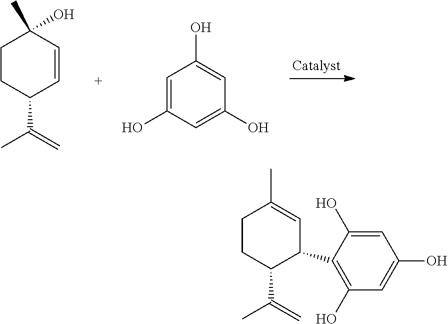

The mother liquor from Example 1 contained approximately 5% of 2-((1S,6R)-3-methyl-6-(prop-1-en-2-yl)cyclohex-2-enyl)benzene-1,3,5-triol. This was isolated by silica gel chromatography. Yield=2.5 g.

### Example 31. Preparation of 3,5-dihydroxy-4-((1S,6R)-3-methyl-6-(prop-1-en-2-yl)cyclohex-2-enyl)phenyl Trifluoromethanesulfonate

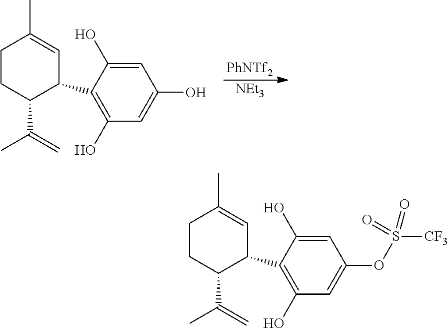 [SMILES: "PhNTf2" ; C=C(C)[C@@H]1CCC(C)=C[C@@H]1c1c(O)cc(O)cc1O ; C=C(C)[C@@H]1CCC(C)=C[C@@H]1c1c(O)cc(OS(=O)(=O)C(F)(F)F)cc1O ; "NEt3"]

Triethylamine (10.8 g, 107 mmol) was added to a mixture of 2-((1S,6R)-3-methyl-6-(prop-1-en-2-yl)cyclohex-2-enyl)benzene-1,3,5-triol (9.35 g, 30.9 mmol) in dichloromethane (100 ml) at room temperature while stirring. Solid N-Phenyl-bis(trifluoromethanesulfonimide) (12.0 g, 33.6 mmol) was added over 1.5 hours and the mixture was kept at room temperature using a water bath. The mixture was stirred at room temperature overnight then quenched with water (40 ml) and the phases separated. The aqueous layer was extracted with dichloromethane (3×25 ml) and the combined organic layers were dried (MgSO₄). It was filtered through a short pad of silica gel and the solvent was removed under reduced pressure. The residue was dissolved in hexanes/CH₂Cl₂ (100 ml of a 1:3 mixture) and filtered through a short silica gel pad, and eluted with hexanes/CH₂Cl₂ (1:3) until no product was detected from the eluent (TLC). The filtrate was evaporated to dryness and the residue was chromatographed to give the product as a white solid. Yield=9.6 grams.

### Example 32. Preparation of 4-((1S,6R)-3-methyl-6-(prop-1-en-2-yl)cyclohex-2-enyl)-3,5-bis(trimethylsilyloxy)phenyl Trifluoromethanesulfonate

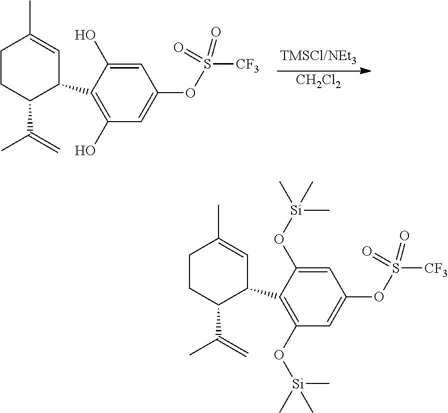

TMSCl (14 g, 128 mol) was added to a mixture of 3,5-dihydroxy-4-((1S,6R)-3-methyl-6-(prop-1-en-2-yl)cyclohex-2-enyl)phenyl trifluoromethanesulfonate (9.5 g, 24 mmol) and NEt₃ (12 g, 120 mmol) in CH₂Cl₂ (60 ml) at 0° C. The mixture was stirred overnight at room temperature. It was filtered and the solids were washed with dichloromethane. The volatiles were removed from the combined filtrate under reduced pressure. The residue was suspended in hexanes (100 ml) and stirred for 2 hours at room temperature. The mixture was filtered and the solvent was removed under reduced pressure, and the residue was dried under vacuum to give the product as a pale yellow oil. Yield=12.3 g.

### Example 33. Preparation of 2-((1S,6R)-3-methyl-6-(prop-1-en-2-yl)cyclohex-2-enyl)-5-pentylbenzene-1,3-diol

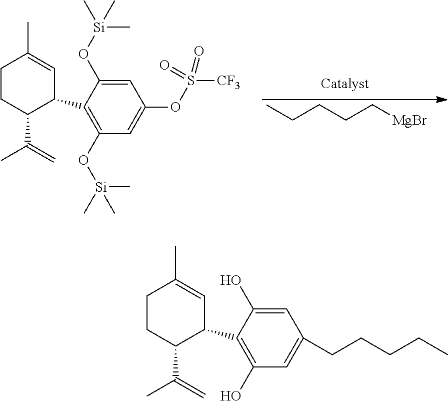

A solution of pentylmagnesium bromide (1.4 ml of a 2.0 M solution in diethyl ether, 2.8 mmol) was added to a mixture of ZnBr₂ (0.63 g, 2.8 mmol) and LiBr (0.3 g, 3.4 mmol) in THF (4 ml) and the suspension was stirred for 30 minutes under argon. A solution of 4-((1S,6R)-3-methyl-6-(prop-1-en-2-yl)cyclohex-2-enyl)-3,5-bis(trimethylsilyloxy)phenyl trifluoromethanesulfonate (1.0 g, 1.86 mmol) and PdCl₂(dppf) (14 mg, 0.019 mmol) in THF (4 ml) was added and the mixture was stirred at room temperature for 12 hours under argon. Water (2 ml) was added followed by 2M H₂SO₄ (1.0 ml) and the mixture stirred at room temperature for 1 hour. The phases were separated and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale yellow oil. Yield=0.55 g.

### Example 34. Preparation of (6aR,10aS)-6,6,9-trimethyl-3-pentyl-6a,7,8,10a-tetrahydro-6H-benzo[c]chromen-1-ol

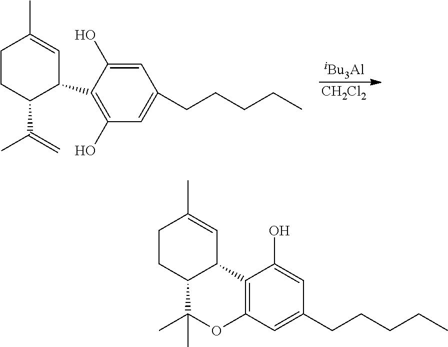

A solution of triisobutylaluminum (0.15 ml of a 1.0 M solution in hexanes, 0.15 mmol) was added to a solution of 2-((1S,6R)-3-methyl-6-(prop-1-en-2-yl)cyclohex-2-enyl)-5-pentylbenzene-1,3-diol (503 mg, 1.6 mmol) in dichloromethane (10 ml) and the mixture was stirred at room temperature for 24 hours. The reaction was quenched with ammonium chloride solution and diethyl ether was added. The phases were separated and the organic layer was dried (MgSO₄), filtered and evaporated to dryness to give the product as a pale yellow resin. Yield=432 mg.

### Example 35. Preparation of 5-icosyl-2-((1S,6R)-3-methyl-6-(prop-1-en-2-yl)cyclohex-2-enyl)benzene-1,3-diol

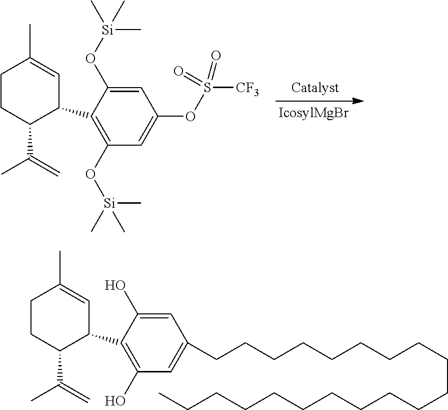

This was prepared according to the procedure outlined in Example 33 and using icosylmagnesium bromide. The product was isolated as a pale yellow solid. Yield=0.86 g.

### Example 36. Preparation of 2-((1S,6R)-3-methyl-6-(prop-1-en-2-yl)cyclohex-2-enyl)-5-phenethylbenzene-1,3-diol

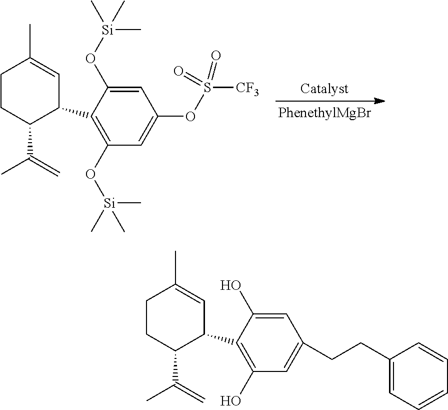 [SMILES: C=C(C)[C@@H]1CCC(C)=C[C@@H]1c1c(O[Si](C)(C)C)cc(OS(=O)(=O)C(F)(F)F)cc1O[Si](C)(C)C ; C=C(C)[C@@H]1CCC(C)=C[C@@H]1c1c(O)cc(CCc2ccccc2)cc1O]

A solution of phenethylmagnesium bromide (1.4 ml of a 2.0 M solution in diethyl ether, 2.8 mmol) was added to a mixture of ZnBr₂ (0.63 g, 2.8 mmol) and LiBr (0.3 g, 3.4 mmol) in THF (4 ml) and the suspension was stirred for 30 minutes under argon. A solution of 4-((1S,6R)-3-methyl-6-(prop-1-en-2-yl)cyclohex-2-enyl)-3,5-bis(trimethylsilyloxy)phenyl trifluoromethanesulfonate (1.0 g, 1.86 mmol) and PdCl₂(dppf) (14 mg, 0.019 mmol) in THF (4 ml) was added and the mixture was stirred at 50° C. for 24 hours under argon. Water (2 ml) was added followed by 2M H₂SO₄ (1.0 ml) and the mixture stirred at room temperature for 1 hour. The phases were separated and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale yellow oil which was purified by chromatography. Yield=0.54 g.

### Example 37. Preparation of (6aR,10aS)-3-icosyl-6,6,9-trimethyl-6a,7,8,10a-tetrahydro-6H-benzo[c]chromen-1-ol

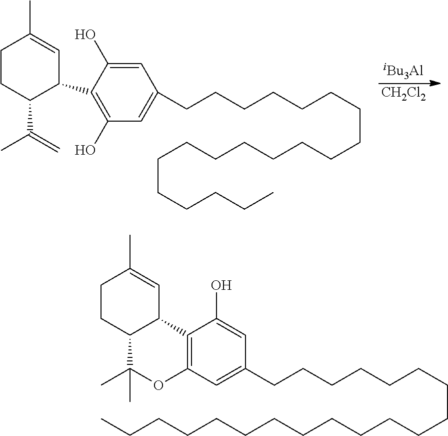

This was prepared according to the procedure outlined in Example 34 and using 5-icosyl-2-((1S,6R)-3-methyl-6-(prop-1-en-2-yl)cyclohex-2-enyl)benzene-1,3-diol (840 mg, 1.6 mmol). The product was isolated as a white solid. Yield=735 mg.

### Example 38. Preparation of (6aR,10aS)-6,6,9-trimethyl-3-phenethyl-6a,7,8,10a-tetrahydro-6H-benzo[c]chromen-1-ol

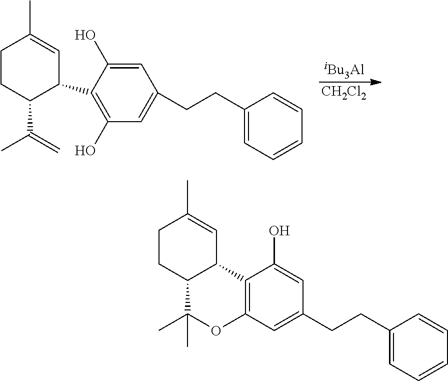

This was prepared according to the procedure outlined in Example 34 and using 2-((1S,6R)-3-methyl-6-(prop-1-en-2-yl)cyclohex-2-enyl)-5-phenethylbenzene-1,3-diol (558 mg, 1.6 mmol). The product was isolated as a pale yellow resin. Yield=450 mg.

### Example 39. Preparation of 2-((1S,6S)-3-methyl-6-(prop-1-en-2-yl)cyclohex-2-enyl)benzene-1,3,5-triol

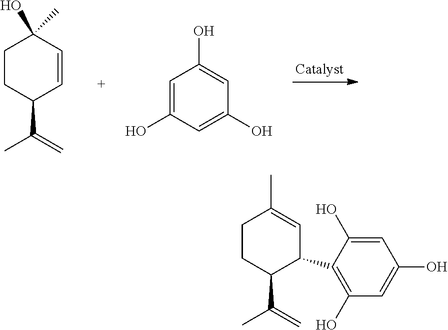

This was prepared according to the procedure described in Example 1 using 1,3,5-trihydroxybenzene and (1R,4S)-1-methyl-4-(prop-1-en-2-yl)cyclohex-2-enol.

### Example 40. Preparation of 3,5-dihydroxy-4-((1S,6S)-3-methyl-6-(prop-1-en-2-yl)cyclohex-2-enyl)phenyl Trifluoromethanesulfonate

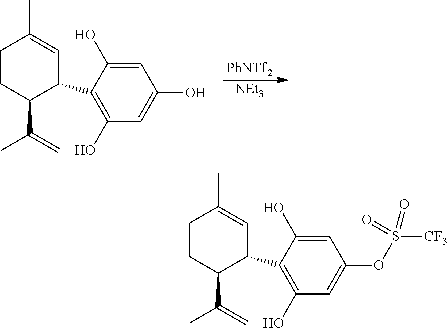 [SMILES: "PhNTf2" ; C=C(C)[C@H]1CCC(C)=C[C@@H]1c1c(O)cc(O)cc1O ; C=C(C)[C@H]1CCC(C)=C[C@@H]1c1c(O)cc(OS(=O)(=O)C(F)(F)F)cc1O ; "NEt3"]

This was prepared according to the procedure described in Example 2 using 2-((1S,6S)-3-methyl-6-(prop-1-en-2-yl)cyclohex-2-enyl)benzene-1,3,5-triol.

### Example 41. Preparation of 4-((1S,6S)-3-methyl-6-(prop-1-en-2-yl)cyclohex-2-enyl)-3,5-bis(trimethylsilyloxy)phenyl Trifluoromethanesulfonate

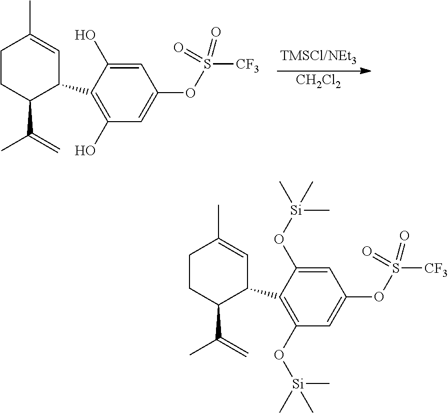

This was prepared according to the procedure described in Example 3 using 3,5-dihydroxy-4-((1S,6S)-3-methyl-6-(prop-1-en-2-yl)cyclohex-2-enyl)phenyl trifluoromethanesulfonate.

### Example 42. Preparation of 2-((1S,6S)-3-methyl-6-(prop-1-en-2-yl)cyclohex-2-enyl)-5-pentylbenzene-1,3-diol (S,S-Cannabidiol)

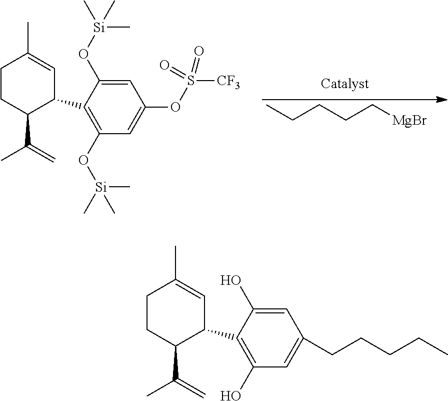

This was prepared according to the procedure described in Example 4 using 4-((1S,6S)-3-methyl-6-(prop-1-en-2-yl)cyclohex-2-enyl)-3,5-bis(trimethylsilyloxy)phenyl trifluoromethanesulfonate.

### Example 43. Preparation of 2-((1S,6S)-3-methyl-6-(prop-1-en-2-yl)cyclohex-2-enyl)-5-propylbenzene-1,3-diol (S,S-Cannabidivarin)

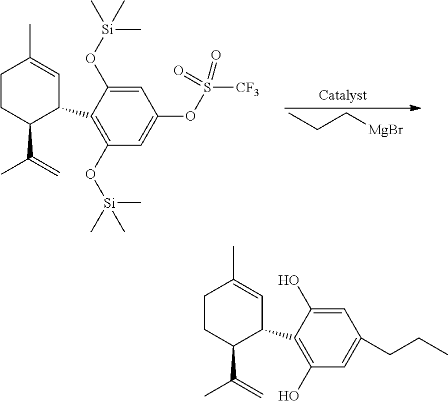

This was prepared according to the procedure described in Example 8 using 4-((1S,6S)-3-methyl-6-(prop-1-en-2-yl)cyclohex-2-enyl)-3,5-bis(trimethylsilyloxy)phenyl trifluoromethanesulfonate.

### Example 44. Preparation of 5-butyl-2-((1S,6S)-3-methyl-6-(prop-1-en-2-yl)cyclohex-2-enyl)benzene-1,3-diol (S,S-Cannabidibutol)

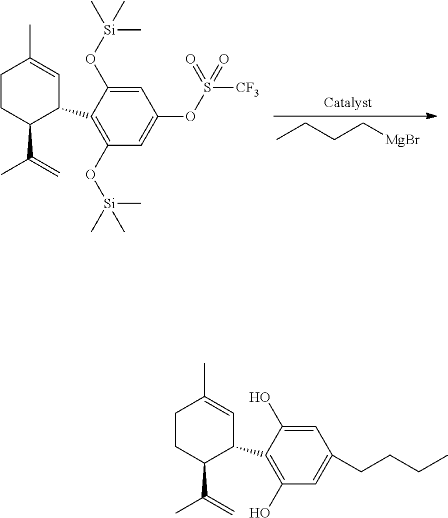

This was prepared according to the procedure described in Example 43 using butylmagnesium bromide.

### Example 45. Preparation of 5-hexyl-2-((1S,6S)-3-methyl-6-(prop-1-en-2-yl)cyclohex-2-enyl)benzene-1,3-diol

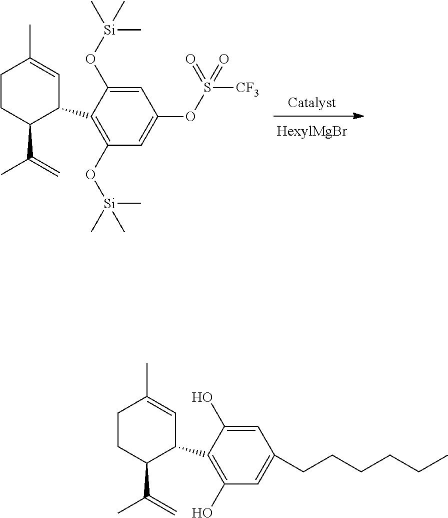 [SMILES: C=C(C)[C@H]1CCC(C)=C[C@@H]1c1c(O[Si](C)(C)C)cc(OS(=O)(=O)C(F)(F)F)cc1O[Si](C)(C)C ; C=C(C)[C@H]1CCC(C)=C[C@@H]1c1c(O)cc(CCCCCC)cc1O]

This was prepared according to the procedure outlined in Example 43 and using hexylmagnesium bromide.

### Example 46. Preparation of 5-heptyl-2-((1S,6S)-3-methyl-6-(prop-1-en-2-yl)cyclohex-2-enyl)benzene-1,3-diol (S,S-Cannabidiphorol)

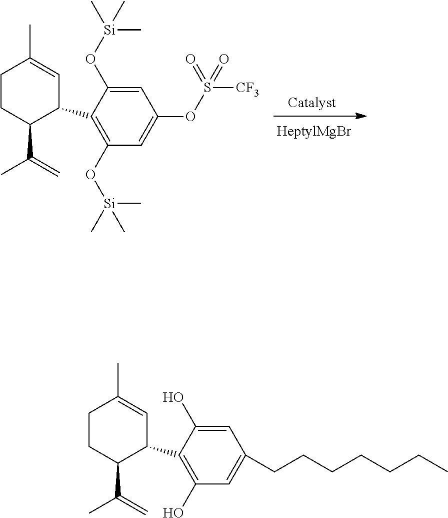 [SMILES: C=C(C)[C@H]1CCC(C)=C[C@@H]1c1c(O[Si](C)(C)C)cc(OS(=O)(=O)C(F)(F)F)cc1O[Si](C)(C)C ; C=C(C)[C@H]1CCC(C)=C[C@@H]1c1c(O)cc(CCCCCCC)cc1O]

This was prepared according to the procedure outlined in Example 43 and using heptylmagnesium bromide.

### Example 47. Preparation of 2-((1S,6S)-3-methyl-6-(prop-1-en-2-yl)cyclohex-2-enyl)-5-phenethylbenzene-1,3-diol

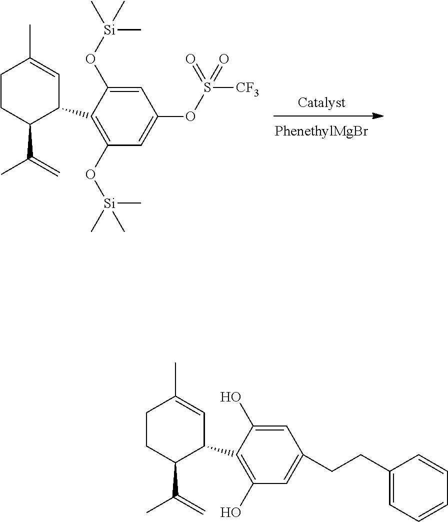 [SMILES: C=C(C)[C@H]1CCC(C)=C[C@@H]1c1c(O[Si](C)(C)C)cc(OS(=O)(=O)C(F)(F)F)cc1O[Si](C)(C)C ; C=C(C)[C@H]1CCC(C)=C[C@@H]1c1c(O)cc(CCc2ccccc2)cc1O]

This was prepared according to the procedure outlined in Example 43 and using phenethylmagnesium bromide.

### Example 48. Preparation of (6aS,10aS)-6,6,9-trimethyl-3-pentyl-6a,7,8,10a-tetrahydro-6H-benzo[c]chromen-1-ol (S,S-Tetrahydrocannabinol)

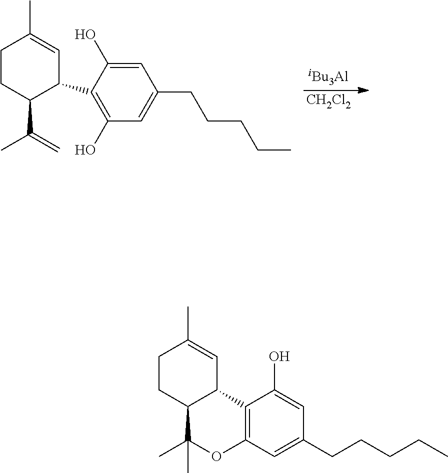 [SMILES: "iBu3Al" ; C=C(C)[C@H]1CCC(C)=C[C@@H]1c1c(O)cc(CCCCC)cc1O ; "CH2Cl2" ; CCCCCc1cc(O)c2c(c1)OC(C)(C)[C@H]1CCC(C)=C[C@H]21]

This was prepared according to the procedure outlined in Example 5 and using 2-((1S,6S)-3-methyl-6-(prop-1-en-2-yl)cyclohex-2-enyl)-5-pentylbenzene-1,3-diol.

### Example 49. Preparation of (6aS,10aS)-6,6,9-trimethyl-3-propyl-6a,7,8,10a-tetrahydro-6H-benzo[c]chromen-1-ol (S,S-Tetrahydrocannabivarin)

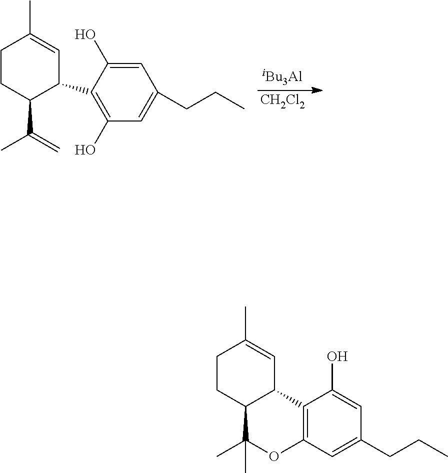

This was prepared according to the procedure outlined in Example 21 and using 2-((1S,6S)-3-methyl-6-(prop-1-en-2-yl)cyclohex-2-enyl)-5-propylbenzene-1,3-diol.

### Example 50. Preparation of (6aS,10aS)-3-butyl-6,6,9-trimethyl-6a,7,8,10a-tetrahydro-6H-benzo[c]chromen-1-ol (S,S-Tetrahydrocannabutol)

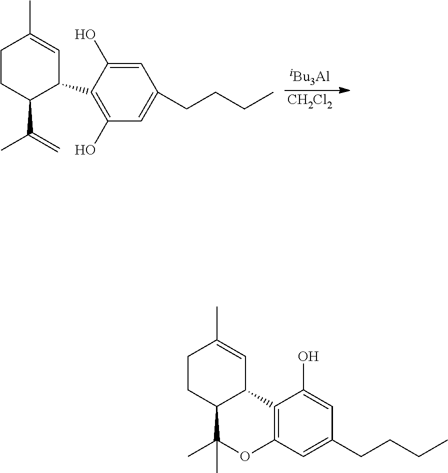

This was prepared according to the procedure outlined in Example 22 and using 5-butyl-2-((1S,6S)-3-methyl-6-(prop-1-en-2-yl)cyclohex-2-enyl)benzene-1,3-diol.

### Example 51. Preparation of (6aS,10aS)-3-hexyl-6,6,9-trimethyl-6a,7,8,10a-tetrahydro-6H-benzo[c]chromen-1-ol

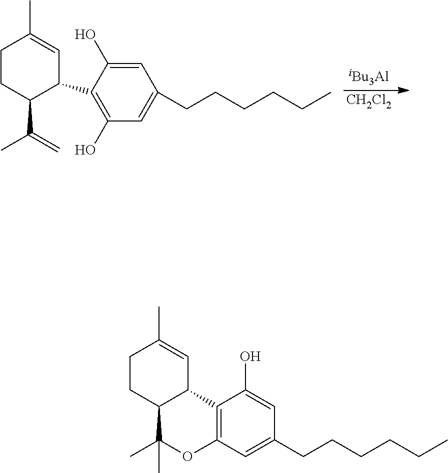

This was prepared according to the procedure outlined in Example 23 and using 5-hexyl-2-((1S,6S)-3-methyl-6-(prop-1-en-2-yl)cyclohex-2-enyl)benzene-1,3-diol.

### Example 52. Preparation of (6aS,10aS)-3-heptyl-6,6,9-trimethyl-6a,7,8,10a-tetrahydro-6H-benzo[c]chromen-1-ol (S,S-Tetrahydrocannabiphorol)

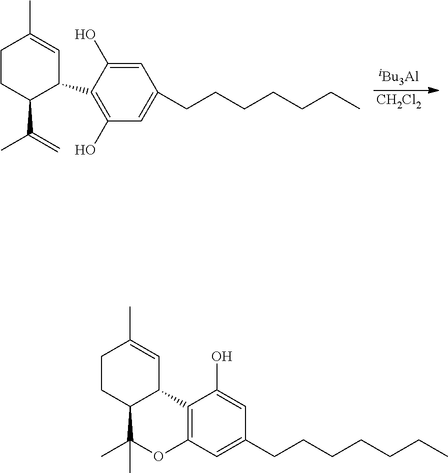

This was prepared according to the procedure outlined in Example 24 and using 5-heptyl-2-((1S,6S)-3-methyl-6-(prop-1-en-2-yl)cyclohex-2-enyl)benzene-1,3-diol.

### Example 53. Preparation of (6aS,10aS)-6,6,9-trimethyl-3-phenethyl-6a,7,8,10a-tetrahydro-6H-benzo[c]chromen-1-ol

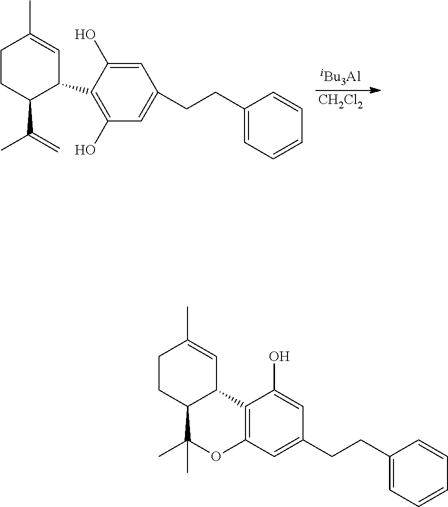

This was prepared according to the procedure outlined in Example 29 and using 2-((1S,6S)-3-methyl-6-(prop-1-en-2-yl)cyclohex-2-enyl)-5-phenethylbenzene-1,3-diol.

### Example 54. Preparation of 2-((1R,6S)-3-methyl-6-(prop-1-en-2-yl)cyclohex-2-enyl)benzene-1,3,5-triol

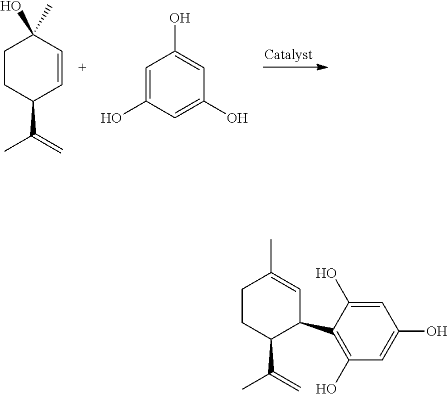 [SMILES: C=C(C)[C@@H]1C=C[C@](C)(O)CC1 ; Oc1cc(O)cc(O)c1 ; C=C(C)[C@H]1CCC(C)=C[C@H]1c1c(O)cc(O)cc1O]

The mother liquor from Example 39 contained approximately 5% of 2-((1R,6S)-3-methyl-6-(prop-1-en-2-yl)cyclohex-2-enyl)benzene-1,3,5-triol. This was isolated using the procedure described in Example 30.

### Example 55. Preparation of 3,5-dihydroxy-4-((1R,6S)-3-methyl-6-(prop-1-en-2-yl)cyclohex-2-enyl)phenyl Trifluoromethanesulfonate

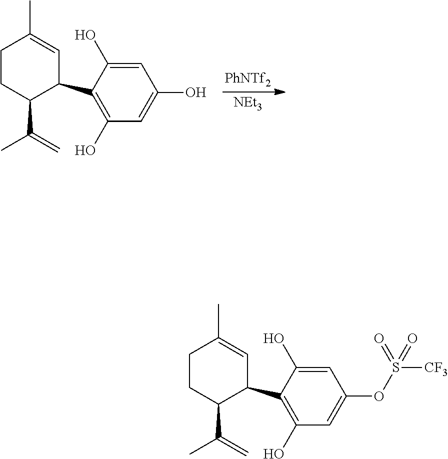 [SMILES: "PhNTf2" ; C=C(C)[C@H]1CCC(C)=C[C@H]1c1c(O)cc(O)cc1O ; C=C(C)[C@H]1CCC(C)=C[C@H]1c1c(O)cc(OS(=O)(=O)C(F)(F)F)cc1O ; "NEt3"]

This was prepared from 2-((1R,6S)-3-methyl-6-(prop-1-en-2-yl)cyclohex-2-enyl)benzene-1,3,5-triol using the procedure described in Example 31.

### Example 56. Preparation of 4-((1R,6S)-3-methyl-6-(prop-1-en-2-yl)cyclohex-2-enyl)-3,5-bis(trimethylsilyloxy)phenyl Trifluoromethanesulfonate

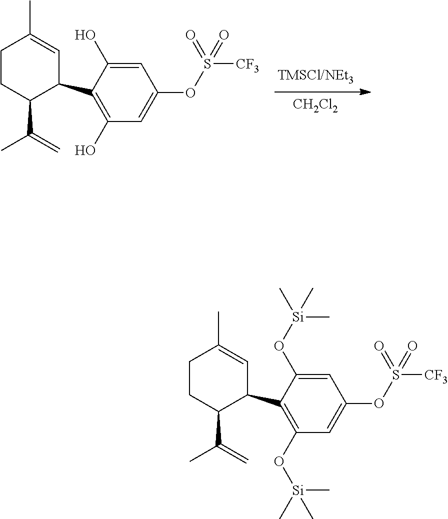

This was prepared from 3,5-dihydroxy-4-((1R,6S)-3-methyl-6-(prop-1-en-2-yl)cyclohex-2-enyl)phenyl trifluoromethanesulfonate using the procedure described in Example 32.

### Example 57. Preparation of 2-((1R,6S)-3-methyl-6-(prop-1-en-2-yl)cyclohex-2-enyl)-5-pentylbenzene-1,3-diol

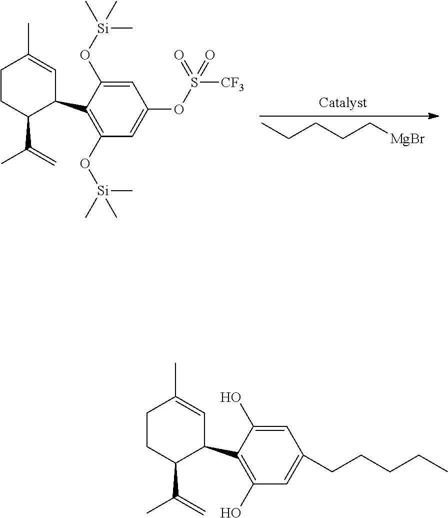

This was prepared from 4-((1R,6S)-3-methyl-6-(prop-1-en-2-yl)cyclohex-2-enyl)-3,5-bis(trimethylsilyloxy)phenyl trifluoromethanesulfonate using the procedure described in Example 33.

### Example 58. Preparation of (6aS,10aR)-6,6,9-trimethyl-3-pentyl-6a,7,8,10a-tetrahydro-6H-benzo[c]chromen-1-ol

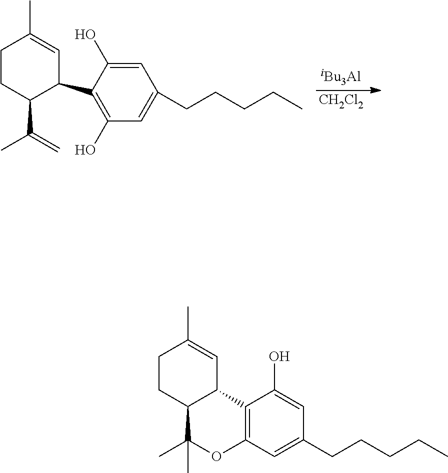

This was prepared from 2-((1R,6S)-3-methyl-6-(prop-1-en-2-yl)cyclohex-2-enyl)-5-pentylbenzene-1,3-diol using the procedure described in Example 34.

### Example 59. Preparation of 5-icosyl-2-((1R,6S)-3-methyl-6-(prop-1-en-2-yl)cyclohex-2-enyl)benzene-1,3-diol

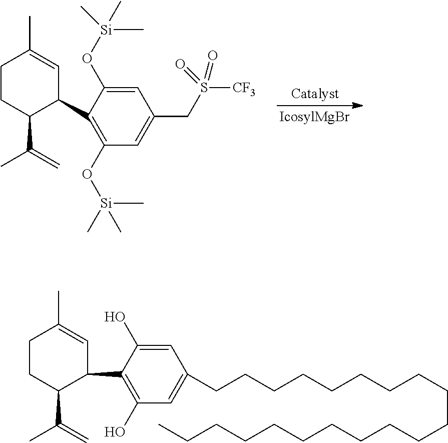 [SMILES: C=C(C)[C@H]1CCC(C)=C[C@H]1c1c(O[Si](C)(C)C)cc(CS(=O)(=O)C(F)(F)F)cc1O[Si](C)(C)C ; C=C(C)[C@H]1CCC(C)=C[C@H]1c1c(O)cc(CCCCCCCCCCCCCCCCCCCC)cc1O]

This was prepared from 4-((1R,6S)-3-methyl-6-(prop-1-en-2-yl)cyclohex-2-enyl)-3,5-bis(trimethylsilyloxy)phenyl trifluoromethanesulfonate using the procedure described in Example 35.

### Example 60. Preparation of 2-((1R,6S)-3-methyl-6-(prop-1-en-2-yl)cyclohex-2-enyl)-5-phenethylbenzene-1,3-diol

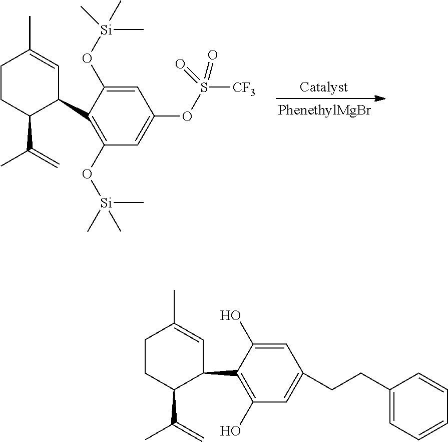 [SMILES: C=C(C)[C@H]1CCC(C)=C[C@H]1c1c(O[Si](C)(C)C)cc(OS(=O)(=O)C(F)(F)F)cc1O[Si](C)(C)C ; C=C(C)[C@H]1CCC(C)=C[C@H]1c1c(O)cc(CCc2ccccc2)cc1O]

This was prepared from 4-((1R,6S)-3-methyl-6-(prop-1-en-2-yl)cyclohex-2-enyl)-3,5-bis(trimethylsilyloxy)phenyl trifluoromethanesulfonate using the procedure described in Example 36.

### Example 61. Preparation of (6aS,10aR)-3-icosyl-6,6,9-trimethyl-6a,7,8,10a-tetrahydro-6H-benzo[c]chromen-1-ol

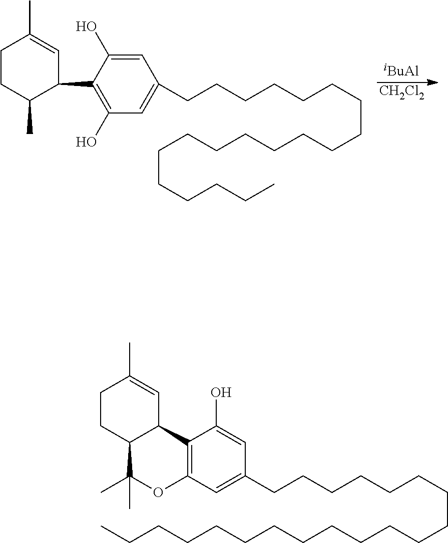

This was prepared according to the procedure outlined in Example 37 and using 5-icosyl-2-((1R,6S)-3-methyl-6-(prop-1-en-2-yl)cyclohex-2-enyl)benzene-1,3-diol.

### Example 62. Preparation of (6aS,10aR)-6,6,9-trimethyl-3-phenethyl-6a,7,8,10a-tetrahydro-6H-benzo[c]chromen-1-ol (Perrottetinene)

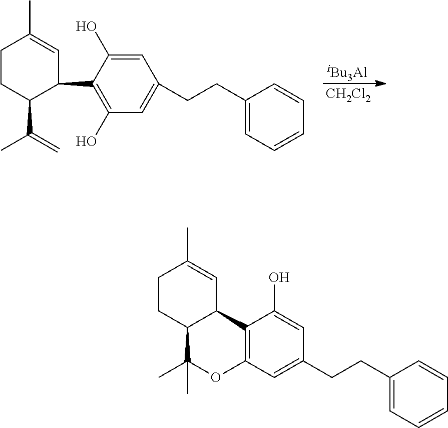

This was prepared according to the procedure outlined in Example 38 and using 2-((1R,6S)-3-methyl-6-(prop-1-en-2-yl)cyclohex-2-enyl)-5-phenethylbenzene-1,3-diol.

### Example 63. Preparation of 3,5-dimethoxy-4-((1R,6R)-3-methyl-6-(prop-1-en-2-yl)cyclohex-2-enyl)phenyl Trifluoromethanesulfonate

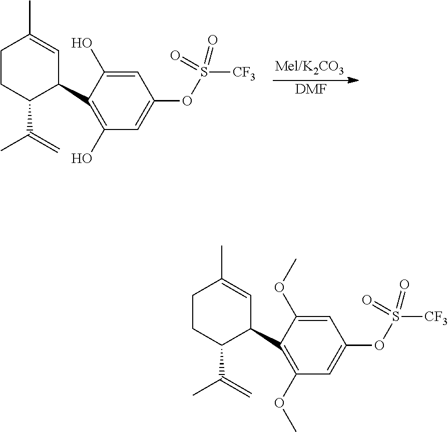

Anhydrous DMF (25 ml) was added to a mixture of 3,5-dihydroxy-4-((1R,6R)-3-methyl-6-(prop-1-en-2-yl)cyclohex-2-enyl)phenyl trifluoromethanesulfonate (5.0 g, 12.7 mmol), methyl iodide (3.77 g, 26.5 mmol), and potassium carbonate (4.2 g, 30.4 mmol) in a Schlenk flask and the suspension stirred vigorously under argon for 12 hours at room temperature. Water (100 ml) was added and the mixture was extracted with ethyl acetate (3×25 ml). The organic layer was washed with water, brine, and dried (MgSO₄). It was filtered and the solvent removed under reduced pressure. The residue was chromatographed using hexanes/CH₂Cl₂ and the pure product was isolated as a yellow oil. Yield=4.3 grams.

### Example 64. Reaction of 3,5-dimethoxy-4-((1R,6R)-3-methyl-6-(prop-1-en-2-yl)cyclohex-2-enyl)phenyl Trifluoromethanesulfonate with n-Pentylzinc Bromide

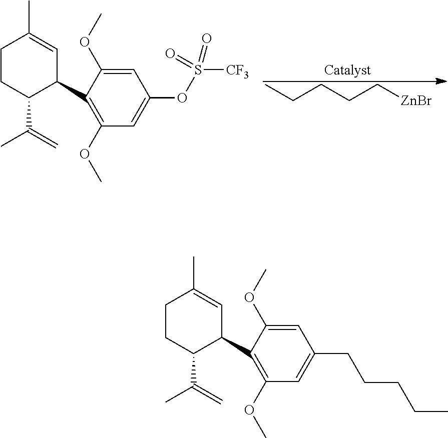

A solution of n-pentylzinc bromide (5.0 ml of a 0.5 M solution in THF, 2.50 mmol) was added to a mixture of 3,5-dimethoxy-4-((1R,6R)-3-methyl-6-(prop-1-en-2-yl)cyclohex-2-enyl)phenyl trifluoromethanesulfonate (1.0 g, 2.38 mmol) and PdCl₂(dppf) (40 mg, 0.06 mmol, 2.5%) and the mixture stirred at room temperature for 1 hour under argon. It was quenched with ammonium chloride solution and diethyl ether added. The phases were separated and the organic layer was dried (MgSO₄), filtered and evaporated to dryness. The NMR spectrum of the residue shows 100% conversion of the substrate to the product. Flash chromatography using hexanes/ethyl acetate yielded the product as a pale yellow oil. Yield=0.70 g.

### Example 65. Reaction of 3,5-dimethoxy-4-((1R,6R)-3-methyl-6-(prop-1-en-2-yl)cyclohex-2-enyl)phenyl Trifluoromethanesulfonate with n-Propylzinc Bromide

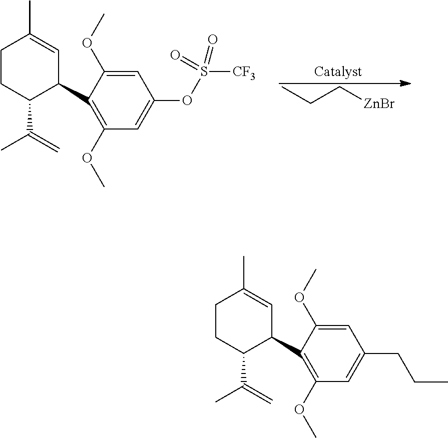

A solution of n-propylzinc bromide (12.0 ml of a 0.5 M solution in THF, 6.0 mmol) was added to a mixture of 3,5-dimethoxy-4-((1R,6R)-3-methyl-6-(prop-1-en-2-yl)cyclohex-2-enyl)phenyl trifluoromethanesulfonate (1.68 g, 4.0 mmol) and PdCl₂(dppf) (30 mg, 0.04 mmol, 1.0%) and the mixture stirred at room temperature for 3 hours under argon. It was quenched with ammonium chloride solution and diethyl ether added. The phases were separated and the organic layer was dried (MgSO₄), filtered and evaporated to dryness. The NMR spectrum of the residue shows 100% conversion of the substrate to the product. Flash chromatography using hexanes/ethyl acetate yielded the product as a pale yellow oil. Yield=1.20 g.

### Example 66. Reaction of 3,5-dimethoxy-4-((1R,6R)-3-methyl-6-(prop-1-en-2-yl)cyclohex-2-enyl)phenyl Trifluoromethanesulfonate with Phenethylzinc Bromide

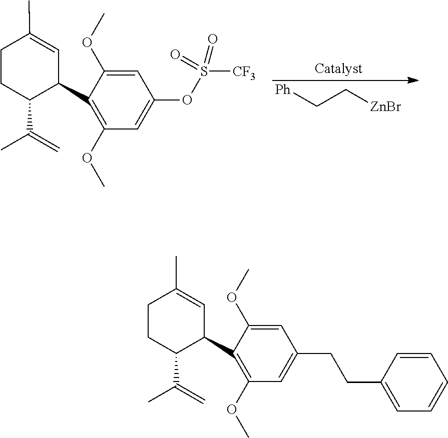

A solution of phenethylzinc bromide (12.0 ml of a 0.5 M solution in THF, 6.0 mmol) was added to a mixture of 3,5-dimethoxy-4-((1R,6R)-3-methyl-6-(prop-1-en-2-yl)cyclohex-2-enyl)phenyl trifluoromethanesulfonate (1.68 g, 4.0 mmol) and PdCl₂(dppf) (30 mg, 0.04 mmol, 1.0%) and the mixture stirred at 50° C. for 24 hours under argon. It was quenched with ammonium chloride solution and diethyl ether added. The phases were separated and the organic layer was dried (MgSO₄), filtered and evaporated to dryness. The NMR spectrum of the residue shows 100% conversion of the substrate to the product. Flash chromatography using hexanes/ethyl acetate yielded the product as a pale yellow oil. Yield=1.42 g.

### Example 67. Preparation of 3,5-dihydroxy-4-((1R,6R)-3-methyl-6-(prop-1-en-2-yl)cyclohex-2-enyl)phenyl 4-methylbenzenesulfonate

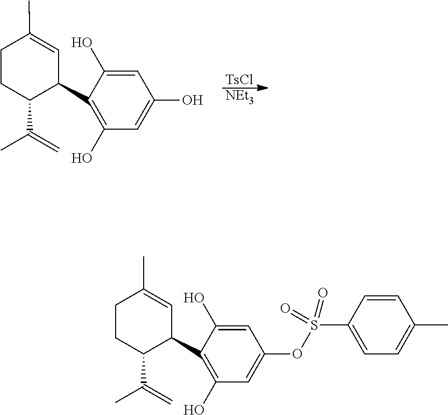 [SMILES: C=C(C)[C@@H]1CCC(C)=C[C@H]1c1c(O)cc(O)cc1O ; "TsCl" ; C=C(C)[C@@H]1CCC(C)=C[C@H]1c1c(O)cc(OS(=O)(=O)c2ccc(C)cc2)cc1O ; "NEt3"]

Triethylamine (31 ml, 222 mmol) was added to a solution of 2-((1R,6R)-3-methyl-6-(prop-1-en-2-yl)cyclohex-2-enyl)benzene-1,3,5-triol (38.5 g, 148 mmol) in dichloromethane (200 ml) and the mixture was cooled to 0° C. A solution of toluenesulfonyl chloride (29.6 g, 155 mmol) was added slowly and the mixture allowed to warm to room temperature and stirred overnight. The reaction was quenched with saturated sodium bicarbonate solution and the phases separated. The aqueous layer was extracted with dichloromethane (3×50 ml) and the combined organic layers dried (MgSO₄), filtered and the solvent removed under reduced pressure. The residue was chromatographed using hexanes/CH₂Cl₂ and the pure product was isolated as a white, crystalline solid. Yield=40.2 grams.

### Example 68. Preparation of 3,5-dihydroxy-4-((1R,6R)-3-methyl-6-(prop-1-en-2-yl)cyclohex-2-enyl)phenyl Trifluoromethanesulfonate

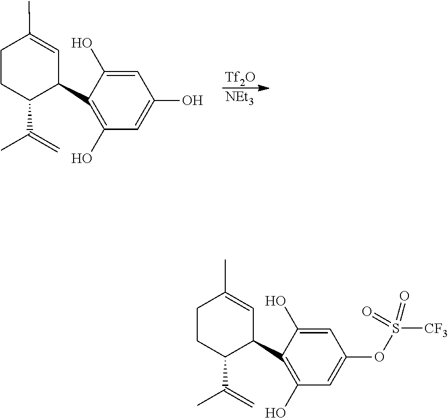 [SMILES: "Tf2O" ; C=C(C)[C@@H]1CCC(C)=C[C@H]1c1c(O)cc(O)cc1O ; C=C(C)[C@@H]1CCC(C)=C[C@H]1c1c(O)cc(OS(=O)(=O)C(F)(F)F)cc1O ; "NEt3"]

Triethylamine (3.1 ml, 22.2 mmol) was added to a solution of 2-((1R,6R)-3-methyl-6-(prop-1-en-2-yl)cyclohex-2-enyl)benzene-1,3,5-triol (3.85 g, 14.8 mmol) in dichloromethane (50 ml) and the mixture was cooled to 0° C. A solution of trifluoromethanesulfonyl anhydride (4.51 g, 16.0 mmol) was added slowly and the mixture allowed to warm to room temperature and stirred overnight. The reaction was quenched with saturated sodium bicarbonate solution and the phases separated. The aqueous layer was extracted with dichloromethane (3×25 ml) and the combined organic layers dried (MgSO₄), filtered and the solvent removed under reduced pressure. The residue was chromatographed using hexanes/CH₂Cl₂ and the pure product was isolated as an orange-red oil. Yield=4.2 grams.

### Example 69. Preparation of 3,5-dimethoxy-4-((1R,6R)-3-methyl-6-(prop-1-en-2-yl)cyclohex-2-enyl)phenyl 4-methylbenzenesulfonate

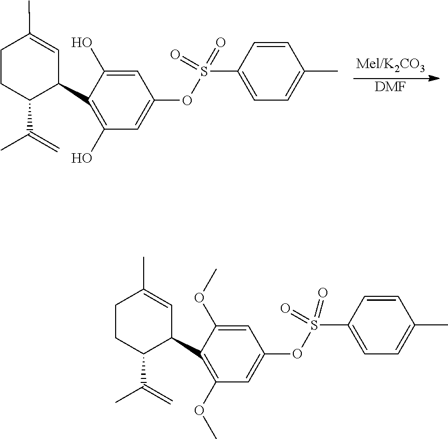

Anhydrous DMF (25 ml) was added to a mixture of 3,5-dihydroxy-4-((1R,6R)-3-methyl-6-(prop-1-en-2-yl)cyclohex-2-enyl)phenyl 4-methylbenzenesulfonate (5.0 g, 12.1 mmol), methyl iodide (3.77 g, 26.5 mmol), and potassium carbonate (4.2 g, 30.4 mmol) in a Schlenk flask and the suspension stirred vigorously under argon for 12 hours at room temperature. Water (100 ml) was added and the mixture was extracted with ethyl acetate (3×25 ml). The organic layer was washed with water, brine, and dried (MgSO₄). It was filtered and the solvent removed under reduced pressure. The residue was chromatographed using hexanes/CH₂Cl₂ and the pure product was isolated as a viscous, pale yellow oil. Yield=4.8 grams.

### Example 70. Preparation of 2-((1R,6R)-3-methyl-6-(prop-1-en-2-yl)cyclohex-2-enyl)-5-(tosyloxy)-1,3-phenylene Diacetate

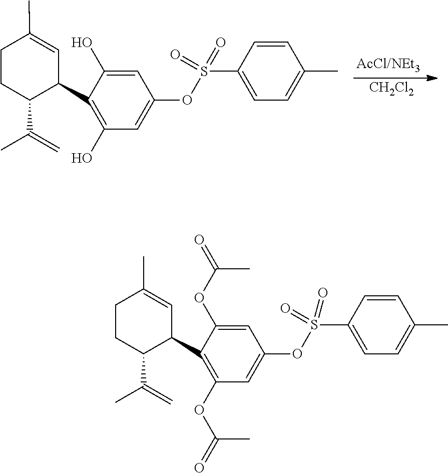

Acetyl chloride (0.39 g, 4.94 mmol) was added to a mixture of 3,5-dihydroxy-4-((1R,6R)-3-methyl-6-(prop-1-en-2-yl)cyclohex-2-enyl)phenyl 4-methylbenzenesulfonate (1.0 g, 2.41 mmol) and NEt₃ (0.73 g, 7.24 mmol) in CH₂Cl₂ (10 ml) at 0° C. under argon. The mixture was stirred at room temperature for 4 hours. The reaction was quenched with water and the phases separated. The aqueous layer was extracted with dichloromethane (3×10 ml) and the combined organic layers washed with dilute sodium bicarbonate solution, then dried (MgSO₄), filtered and the solvent removed under reduced pressure. The residue was chromatographed using hexanes/CH₂Cl₂ and the pure product was isolated as a pale yellow oil. Yield=1.12 grams.

### Example 71. Preparation of 2-((1R,6R)-3-methyl-6-(prop-1-en-2-yl)cyclohex-2-enyl)-5-(trifluoromethylsulfonyloxy)-1,3-phenylene Diacetate

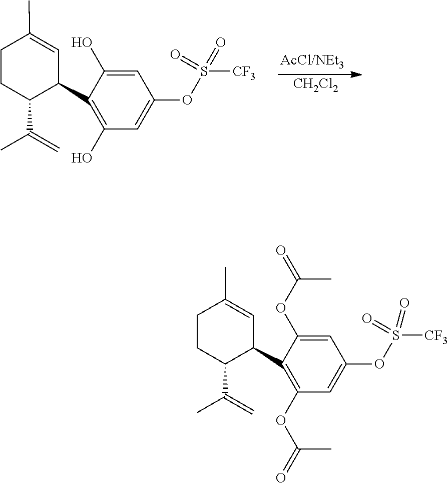

Acetyl chloride (2.05 g, 26.1 mmol) was added to a mixture of 3,5-dihydroxy-4-((1R,6R)-3-methyl-6-(prop-1-en-2-yl)cyclohex-2-enyl)phenyl trifluoromethanesulfonate (5.0 g, 12.7 mmol) and NEt₃ (3.86 g, 38.2 mmol) in CH₂Cl₂ (50 ml) at 0° C. under argon. The mixture was stirred at room temperature for 15 hours. Another portion of acetyl chloride (2.0 g) was added and the reaction was stirred at room temperature until completion (TLC). The reaction was quenched with sodium bicarbonate solution and the phases separated. The aqueous layer was extracted with dichloromethane (3×25 ml) and the combined organic layers washed with brine, then dried (MgSO₄), filtered and the solvent removed under reduced pressure. The residue was chromatographed using hexanes/CH₂Cl₂ and the pure product was isolated as a pale yellow oil. Yield=5.51 grams.

### Example 72. Preparation of 4-((1R,6R)-3-methyl-6-(prop-1-en-2-yl)cyclohex-2-enyl)-3,5-bis(trimethylsilyloxy)phenyl 4-methyl Benzenesulfonate

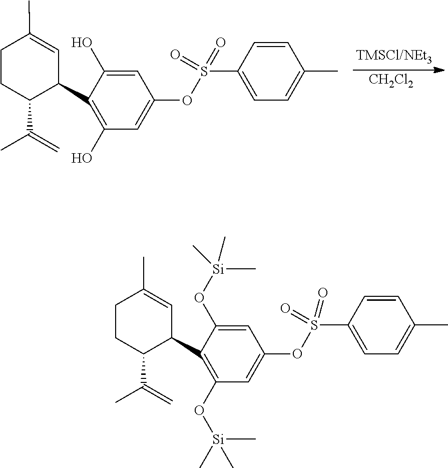

Trimethylsilyl chloride (2.5 g, 23.0 mmol) was added to a mixture of 3,5-dihydroxy-4-((1R,6R)-3-methyl-6-(prop-1-en-2-yl)cyclohex-2-enyl)phenyl 4-methylbenzenesulfonate (3.0 g, 7.2 mmol) and NEt₃ (2.8 g, 27.7 mmol) in CH₂Cl₂ (25 ml) at 0° C. under argon. The mixture was stirred at room temperature for 12 hours. It was filtered and the solvent was removed from the filtrate. It was then suspended in hexanes (25 ml) and stirred for 4 hours. It was filtered and the solvent removed under reduced pressure and the product dried under vacuum to give a yellow brown oil. Yield=4.00 g.

### Example 73. Reaction of 3,5-dihydroxy-4-((1R,6R)-3-methyl-6-(prop-1-en-2-yl)cyclohex-2-enyl)phenyl Trifluoromethanesulfonate with n-Pentylzinc Bromide

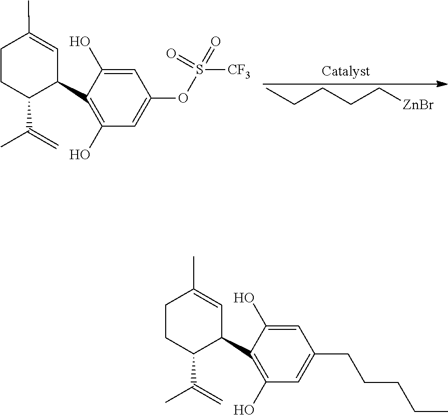

A solution of n-pentylzinc bromide (2.1 ml of a 0.5 M solution in THF, 1.04 mmol) was added to a mixture of 3,5-dihydroxy-4-((1R,6R)-3-methyl-6-(prop-1-en-2-yl)cyclohex-2-enyl)phenyl trifluoromethanesulfonate (100 mg, 0.26 mmol), ZnBr₂ (117 mg, 0.52 mmol) and PdCl₂(dppf) (10 mg, 0.03 mmol, 5%) and the mixture stirred at 60° C. for 12 hours under argon. It was cooled to room temperature and quenched with ammonium chloride solution and diethyl ether added. The phases were separated and the organic layer was dried (MgSO₄), filtered and evaporated to dryness. The NMR spectrum of the residue shows 15% conversion of the substrate to the product.

### Example 74. Reaction of 3,5-dihydroxy-4-((1R,6R)-3-methyl-6-(prop-1-en-2-yl)cyclohex-2-enyl)phenyl Trifluoromethanesulfonate with n-Propylzinc Bromide

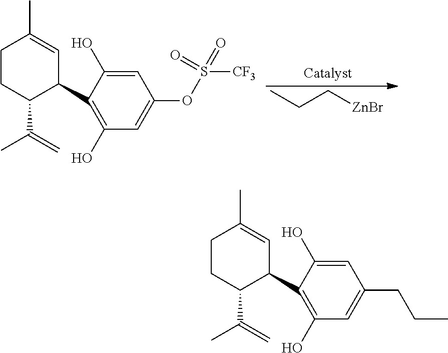

A solution of n-propylzinc bromide (2.1 ml of a 0.5 M solution in THF, 1.04 mmol) was added to a mixture of 3,5-dihydroxy-4-((1R,6R)-3-methyl-6-(prop-1-en-2-yl)cyclohex-2-enyl)phenyl trifluoromethanesulfonate (100 mg, 0.26 mmol), ZnBr₂ (117 mg, 0.52 mmol) and PdCl₂(dppf) (10 mg, 0.03 mmol, 5%) and the mixture stirred at 60° C. for 12 hours under argon. It was cooled to room temperature and quenched with ammonium chloride solution and diethyl ether added. The phases were separated and the organic layer was dried (MgSO₄), filtered and evaporated to dryness. The NMR spectrum of the residue shows 12% conversion of the substrate to the product.

### Example 75. Reaction of 2-((1R,6R)-3-methyl-6-(prop-1-en-2-yl)cyclohex-2-enyl)-5-(trifluoromethylsulfonyloxy)-1,3-phenylene Diacetate with n-Pentyl-Zinc Bromide

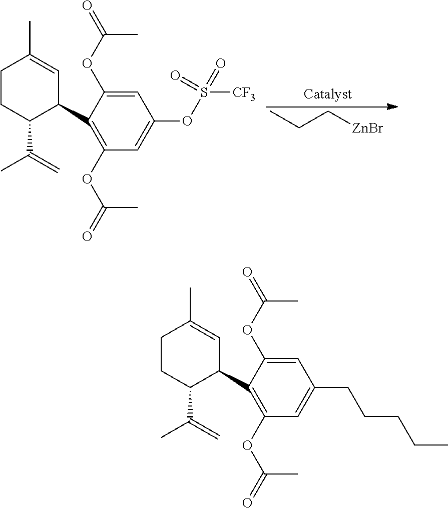

A solution of n-pentylzinc bromide (6.3 ml of a 0.5 M solution in THF, 3.15 mmol) was added to a mixture of 2-((1R,6R)-3-methyl-6-(prop-1-en-2-yl)cyclohex-2-enyl)-5-(trifluoromethylsulfonyloxy)-1,3-phenylene diacetate (1.0 g, 2.10 mmol) and PdCl₂(dppf) (35 mg, 0.05 mmol, 2.3%) and the mixture stirred at 60° C. for 12 hours under argon. It was cooled to room temperature, quenched with ammonium chloride solution and diethyl ether added. The phases were separated and the organic layer was dried (MgSO₄), filtered and evaporated to dryness. The NMR spectrum of the residue shows 100% conversion of the substrate to the product. Flash chromatography using hexanes/ethyl acetate yielded the product as a pale yellow oil. Yield=0.67 g.

### Example 76. Reaction of 2-((1R,6R)-3-methyl-6-(prop-1-en-2-yl)cyclohex-2-enyl)-5-(trifluoromethylsulfonyloxy)-1,3-phenylene Diacetate with n-Propyl-Zinc Bromide

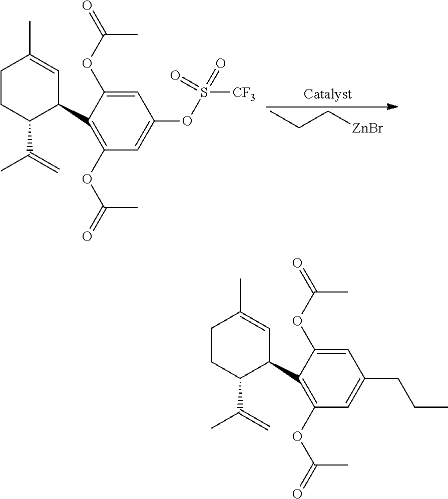

A solution of n-propylzinc bromide (6.3 ml of a 0.5 M solution in THF, 3.15 mmol) was added to a mixture of 2-((1R,6R)-3-methyl-6-(prop-1-en-2-yl)cyclohex-2-enyl)-5-(trifluoromethylsulfonyloxy)-1,3-phenylene diacetate (1.0 g, 2.10 mmol) and PdCl₂(dppf) (35 mg, 0.05 mmol, 2.3%) and the mixture stirred at 60° C. for 12 hours under argon. It was cooled to room temperature, quenched with ammonium chloride solution and diethyl ether added. The phases were separated and the organic layer was dried (MgSO₄), filtered and evaporated to dryness. The NMR spectrum of the residue shows 100% conversion of the substrate to the product. Flash chromatography using hexanes/ethyl acetate yielded the product as a pale yellow oil. Yield=0.65 g.

### Example 77. Preparation of (2-((1R,6R)-3-methyl-6-(prop-1-en-2-yl)cyclohex-2-enyl)-5-pentyl-1,3-phenylene)bis(oxy)bis(trimethylsilane)

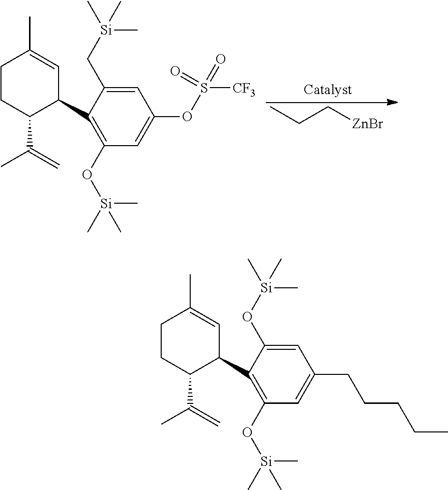

A solution of n-pentylzinc bromide (5.6 ml of a 0.5 M solution in THF, 2.80 mmol) was added to a mixture of 4-((1R,6R)-3-methyl-6-(prop-1-en-2-yl)cyclohex-2-enyl)-3,5-bis(trimethylsilyloxy)phenyl trifluoromethanesulfonate (1.0 g, 1.87 mmol) and PdCl₂(dppf) (34 mg, 0.047 mmol, 2.5%) and the mixture stirred at room temperature for 1 hour under argon. It was quenched with ammonium chloride solution and diethyl ether added. The phases were separated and the organic layer was dried (MgSO₄), filtered and evaporated to dryness. The NMR spectrum of the residue shows 100% conversion of the substrate to the product. Yield=0.83 g.

### Example 78. Preparation of (2-((1R,6R)-3-methyl-6-(prop-1-en-2-yl)cyclohex-2-enyl)-5-propyl-1,3-phenylene)bis(oxy)bis(trimethylsilane)

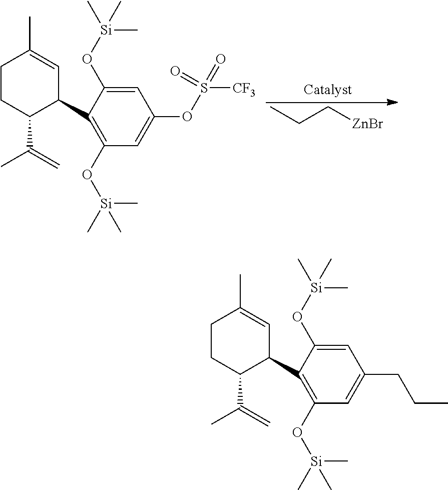

A solution of n-propylzinc bromide (5.6 ml of a 0.5 M solution in THF, 2.80 mmol) was added to a mixture of 4-((1R,6R)-3-methyl-6-(prop-1-en-2-yl)cyclohex-2-enyl)-3,5-bis(trimethylsilyloxy)phenyl trifluoromethanesulfonate (1.0 g, 1.87 mmol) and PdCl₂(dppf) (34 mg, 0.047 mmol, 2.5%) and the mixture stirred at room temperature for 2 hours under argon. It was quenched with ammonium chloride solution and diethyl ether added. The phases were separated and the organic layer was dried (MgSO₄), filtered and evaporated to dryness. The NMR spectrum of the residue shows 100% conversion of the substrate to the product. Yield=0.79 g.

### Example 79. Hydrolysis of (2-((1R,6R)-3-methyl-6-(prop-1-en-2-yl)cyclohex-2-enyl)-5-pentyl-1,3-phenylene)bis(oxy)bis(trimethylsilane)

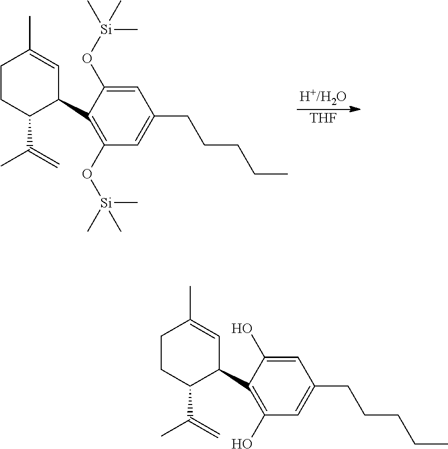 [SMILES: C=C(C)[C@@H]1CCC(C)=C[C@H]1c1c(O[Si](C)(C)C)cc(CCCCC)cc1O[Si](C)(C)C ; C=C(C)[C@@H]1CCC(C)=C[C@H]1c1c(O)cc(CCCCC)cc1O ; "THF"]

Ethanol (10 ml) and dilute H₂SO₄ (5 ml of a 2M solution) was added to a solution of (2-((1R,6R)-3-methyl-6-(prop-1-en-2-yl)cyclohex-2-enyl)-5-pentyl-1,3-phenyl-ene)bis(oxy)bis(trimethylsilane) (0.83 g) in THF (5 ml) and the mixture stirred for 1 hour at room temperature. It was extracted with ether (3×10 ml) and the combined extracts dried (MgSO₄) then evaporated to dryness. The product was purified by flash chromatography using hexanes/ethylacetate. Yield=0.55 g.

### Example 80. Hydrolysis of (2-((1R,6R)-3-methyl-6-(prop-1-en-2-yl)cyclohex-2-enyl)-5-propyl-1,3-phenylene)bis(oxy)bis(trimethylsilane)

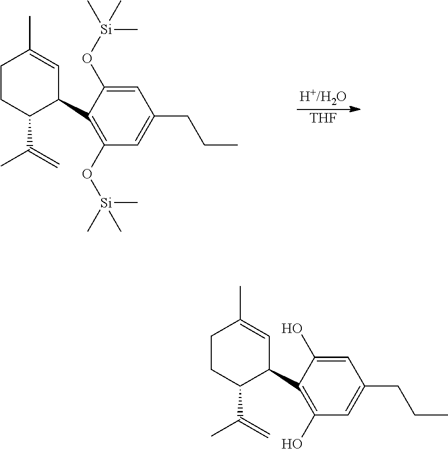 [SMILES: C=C(C)[C@@H]1CCC(C)=C[C@H]1c1c(O[Si](C)(C)C)cc(CCC)cc1O[Si](C)(C)C ; C=C(C)[C@@H]1CCC(C)=C[C@H]1c1c(O)cc(CCC)cc1O ; "THF"]

Ethanol (10 ml) and dilute H₂SO₄ (5 ml of a 2M solution) was added to a solution of (2-((1R,6R)-3-methyl-6-(prop-1-en-2-yl)cyclohex-2-enyl)-5-pentyl-1,3-phenyl-ene)bis(oxy)bis(trimethylsilane) (0.79 g) in THF (5 ml) and the mixture stirred for 1 hour at room temperature. It was extracted with ether (3×10 ml) and the combined extracts dried (MgSO₄) then evaporated to dryness. The product was purified by flash chromatography using hexanes/ethylacetate. Yield=0.53 g.

### Example 81. Preparation of (2-((1R,6R)-3-methyl-6-(prop-1-en-2-yl)cyclohex-2-enyl)-5-pentyl-1,3-phenylene)bis(oxy)bis(trimethylsilane)

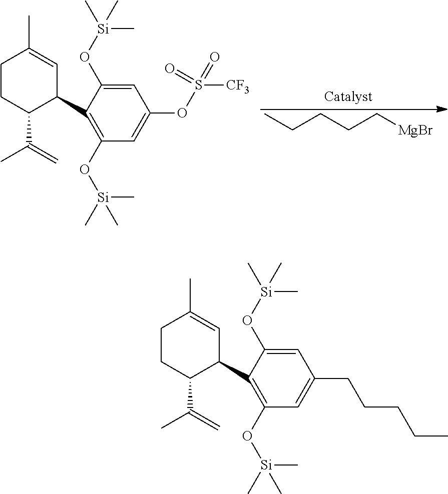

A solution of n-pentylmagnesium bromide (2.0 ml of a 1.0 M solution in diethyl ether, 2.0 mmol) was added to a mixture of 4-((1R,6R)-3-methyl-6-(prop-1-en-2-yl)cyclohex-2-enyl)-3,5-bis(trimethylsilyloxy)phenyl trifluoromethanesulfonate (200 mg, 0.37 mmol) and PdCl₂(dppf) (10 mg, 0.014 mmol) and the mixture stirred at room temperature for 1 hour under argon. It was quenched with ammonium chloride solution and diethyl ether added. The phases were separated and the organic layer was dried (MgSO₄), filtered and evaporated to dryness. The NMR spectrum of the residue shows 80% conversion of the substrate to the product.

### Example 82. Preparation of (2-((1R,6R)-3-methyl-6-(prop-1-en-2-yl)cyclohex-2-enyl)-5-propyl-1,3-phenylene)bis(oxy)bis(trimethylsilane)

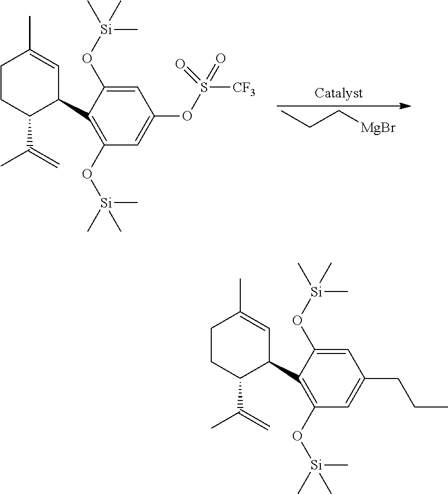

A solution of n-propylmagnesium bromide (2.0 ml of a 1.0 M solution in diethyl ether, 2.0 mmol) was added to a mixture of 4-((1R,6R)-3-methyl-6-(prop-1-en-2-yl)cyclohex-2-enyl)-3,5-bis(trimethylsilyloxy)phenyl trifluoromethanesulfonate (200 mg, 0.37 mmol) and PdCl₂(dppf) (10 mg, 0.014 mmol) and the mixture stirred at room temperature for 2 hours under argon. It was quenched with ammonium chloride solution and diethyl ether added. The phases were separated and the organic layer was dried (MgSO₄), filtered and evaporated to dryness. The NMR spectrum of the residue shows 85% conversion of the substrate to the product.

### Example 83. Reaction of 3,5-dimethoxy-4-((1R,6R)-3-methyl-6-(prop-1-en-2-yl)cyclohex-2-enyl)phenyl 4-methyl Benzenesulfonate with n-Pentylzinc Bromide Using [1,1′-Bis(diphenylphosphino)ferrocene]dichloropalladium(II) Chloride and Zinc Bromide as Catalyst

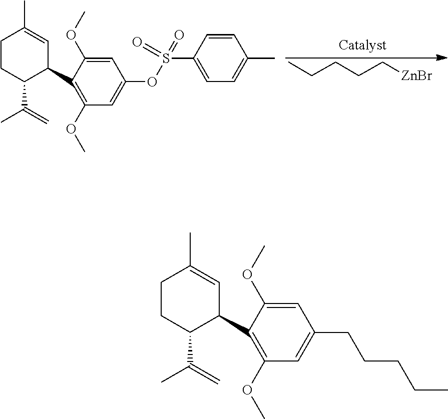

A solution of n-pentylzinc bromide (1.8 ml of a 0.5 M solution in THF, 0.90 mmol) was added to a mixture of 3,5-dimethoxy-4-((1R,6R)-3-methyl-6-(prop-1-en-2-yl)cyclohex-2-enyl)phenyl 4-methylbenzenesulfonate (200 mg, 0.45 mmol), ZnBr₂ (102 mg, 0.45 mmol) and PdCl₂(dppf) (16 mg, 0.022 mmol, 5%) and the mixture stirred at 60° C. for 15 hours under argon. It was cooled to room temperature and quenched with ammonium chloride solution and diethyl ether added. The phases were separated and the organic layer was dried (MgSO₄), filtered and evaporated to dryness. The NMR spectrum of the residue shows 12% conversion of the substrate to the product.

### Example 84. Reaction of 3,5-dimethoxy-4-((1R,6R)-3-methyl-6-(prop-1-en-2-yl)cyclohex-2-enyl)phenyl 4-methyl Benzenesulfonate with n-Pentylzinc Bromide Using [1,1-Bis(diphenylphosphino)ferrocene]dichloropalladium(II) Chloride and Zinc Triflate as Catalyst

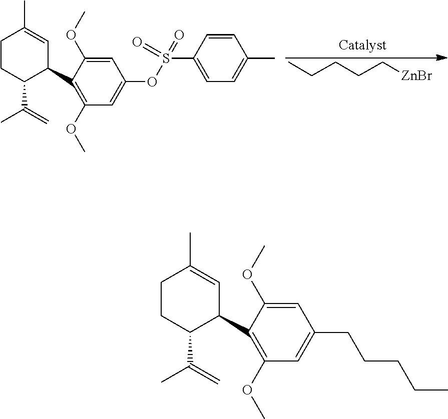

A solution of n-pentylzinc bromide (1.8 ml of a 0.5 M solution in THF, 0.90 mmol) was added to a mixture of 3,5-dimethoxy-4-((1R,6R)-3-methyl-6-(prop-1-en-2-yl)cyclohex-2-enyl)phenyl 4-methylbenzenesulfonate (200 mg, 0.45 mmol), Zn(OTf)₂ (164 mg, 0.45 mmol) and PdCl₂(dppf) (16 mg, 0.022 mmol, 5%) and the mixture stirred at 60° C. for 15 hours under argon. It was cooled to room temperature and quenched with ammonium chloride solution and diethyl ether added. The phases were separated and the organic layer was dried (MgSO₄), filtered and evaporated to dryness. The NMR spectrum of the residue shows 15% conversion of the substrate to the product.

### Example 85. Reaction of 3,5-dimethoxy-4-((1R,6R)-3-methyl-6-(prop-1-en-2-yl)cyclohex-2-enyl)phenyl 4-methyl Benzenesulfonate with n-Pentylzinc Bromide Using [1,1-Bis(diphenylphosphino)ferrocene]dichloropalladium(II) Chloride and Copper(II) Bromide as Catalyst

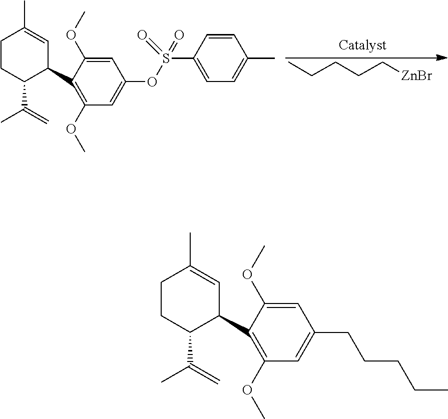

A solution of n-pentylzinc bromide (1.8 ml of a 0.5 M solution in THF, 0.90 mmol) was added to a mixture of 3,5-dimethoxy-4-((1R,6R)-3-methyl-6-(prop-1-en-2-yl)cyclohex-2-enyl)phenyl 4-methylbenzenesulfonate (200 mg, 0.45 mmol), CuBr₂ (101 mg, 0.45 mmol) and PdCl₂(dppf) (16 mg, 0.022 mmol, 5%) and the mixture stirred at 60° C. for 15 hours under argon. It was cooled to room temperature and quenched with ammonium chloride solution and diethyl ether added. The phases were separated and the organic layer was dried (MgSO₄), filtered and evaporated to dryness. The NMR spectrum of the residue shows 26% conversion of the substrate to the product.

### Example 86. Reaction of 3,5-dimethoxy-4-((1R,6R)-3-methyl-6-(prop-1-en-2-yl)cyclohex-2-enyl)phenyl 4-methyl Benzenesulfonate with n-Propylzinc Bromide Using [1,1′-Bis(diphenylphosphino)ferrocene]dichloropalladium(II) Chloride and Zinc Bromide as Catalyst

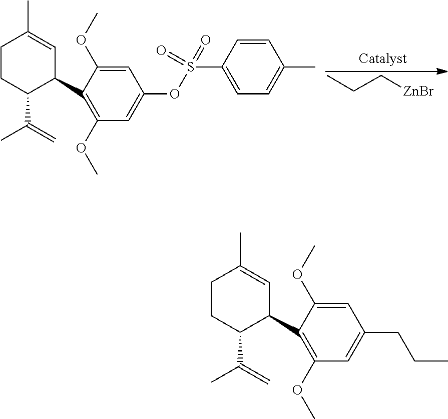

A solution of n-propylzinc bromide (1.8 ml of a 0.5 M solution in THF, 0.90 mmol) was added to a mixture of 3,5-dimethoxy-4-((1R,6R)-3-methyl-6-(prop-1-en-2-yl)cyclohex-2-enyl)phenyl 4-methylbenzenesulfonate (200 mg, 0.45 mmol), ZnBr₂ (102 mg, 0.45 mmol) and PdCl₂(dppf) (16 mg, 0.022 mmol, 5%) and the mixture stirred at 60° C. for 15 hours under argon. It was cooled to room temperature and quenched with ammonium chloride solution and diethyl ether added. The phases were separated and the organic layer was dried (MgSO₄), filtered and evaporated to dryness. The NMR spectrum of the residue shows 15% conversion of the substrate to the product.

### Example 87. Reaction of 3,5-dihydroxy-4-((1R,6R)-3-methyl-6-(prop-1-en-2-yl)cyclohex-2-enyl)phenyl Trifluoromethanesulfonate with n-Pentylmagnesium Bromide Using [1,1′-Bis(diphenylphosphino)ferrocene]dichloro-palladium(II) as Catalyst

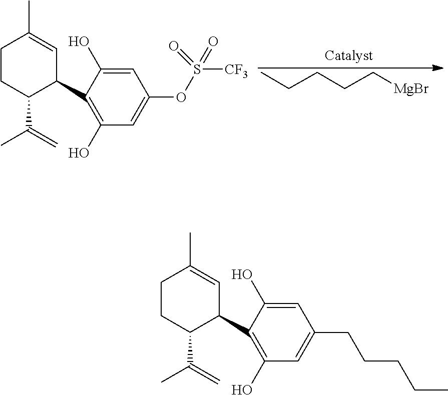

A solution of n-pentylmagnesium bromide (2.0 ml of a 1.0 M solution in diethyl ether, 2.0 mmol) was added to a mixture of 3,5-dihydroxy-4-((1R,6R)-3-methyl-6-(prop-1-en-2-yl)cyclohex-2-enyl)phenyl trifluoromethanesulfonate (200 mg, 0.45 mmol) and PdCl₂(dppf) (10 mg, 0.014 mmol, 3%) and the mixture stirred at 60° C. for 15 hours under argon. It was cooled to room temperature and quenched with ammonium chloride solution and diethyl ether added. The phases were separated and the organic layer was dried (MgSO₄), filtered and evaporated to dryness. The NMR spectrum of the residue shows 20% conversion of the substrate to the product.

### Example 88. Reaction of 3,5-dihydroxy-4-((1R,6R)-3-methyl-6-(prop-1-en-2-yl)cyclohex-2-enyl)phenyl Trifluoromethanesulfonate with n-Propylmagnesium Bromide Using [1,1′-Bis(diphenylphosphino)ferrocene]dichloro-palladium(II) as Catalyst

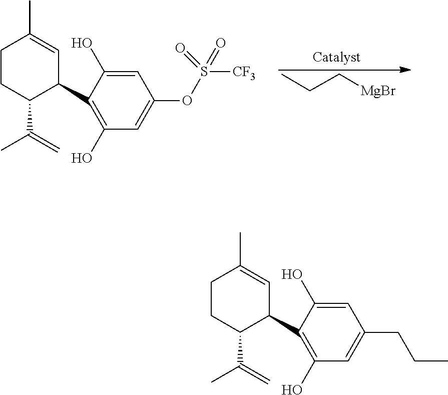

A solution of n-propylmagnesium bromide (2.0 ml of a 1.0 M solution in diethyl ether, 2.0 mmol) was added to a mixture of 3,5-dihydroxy-4-((1R,6R)-3-methyl-6-(prop-1-en-2-yl)cyclohex-2-enyl)phenyl trifluoromethanesulfonate (200 mg, 0.45 mmol) and PdCl₂(dppf) (10 mg, 0.014 mmol, 3%) and the mixture stirred at 60° C. for 15 hours under argon. It was cooled to room temperature and quenched with ammonium chloride solution and diethyl ether added. The phases were separated and the organic layer was dried (MgSO₄), filtered and evaporated to dryness. The NMR spectrum of the residue shows 18% conversion of the substrate to the product.

### Example 89. Reaction of 1,3-dimethoxy-2-((1R,6R)-3-methyl-6-(prop-1-en-2-yl)cyclohex-2-enyl)-5-pentylbenzene with Sodium Ethylthiolate

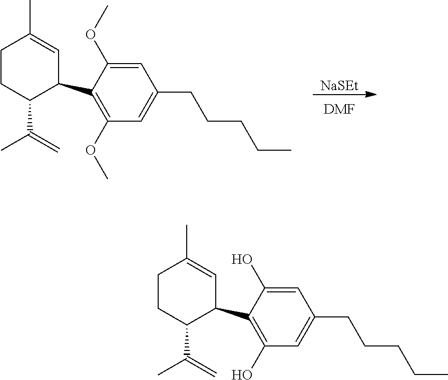

Sodium ethylthiolate (1.33 g, 15.8 mmol) was added to a solution of 1,3-dimethoxy-2-((1R,6R)-3-methyl-6-(prop-1-en-2-yl)cyclohex-2-enyl)-5-pentyl-benzene (1.35 g, 3.94 mmol) in DMF (10 ml) and the mixture heated at 150° C. for 5 hours under argon. It was cooled to room temperature and quenched with ammonium chloride solution and diethyl ether added. The phases were separated and the organic layer was dried (MgSO₄), filtered and evaporated to dryness. The residue was purified by flash chromatography. Yield=1.02 g.

### Example 90. Reaction of 1,3-dimethoxy-2-((1R,6R)-3-methyl-6-(prop-1-en-2-yl)cyclohex-2-enyl)-5-pentylbenzene with Sodium Ethylthiolate

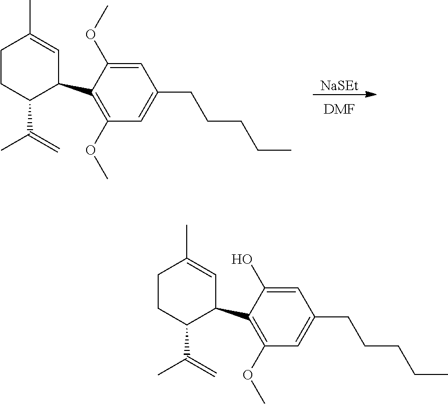

Sodium ethylthiolate (133 mg, 1.6 mmol) was added to a solution of 1,3-dimethoxy-2-((1R,6R)-3-methyl-6-(prop-1-en-2-yl)cyclohex-2-enyl)-5-pentyl-benzene (135 mg, 0.40 mmol) in DMF (5 ml) and the mixture heated at 120° C. for 3 hours under argon. It was cooled to room temperature and quenched with ammonium chloride solution and diethyl ether added. The phases were separated and the organic layer was dried (MgSO₄), filtered and evaporated to dryness. The residue was purified by flash chromatography. Yield=95 mg.

### Example 91. Reaction of 1,3-dimethoxy-2-((1R,6R)-3-methyl-6-(prop-1-en-2-yl)cyclohex-2-enyl)-5-pentylbenzene with Sodium Dodecylthiolate

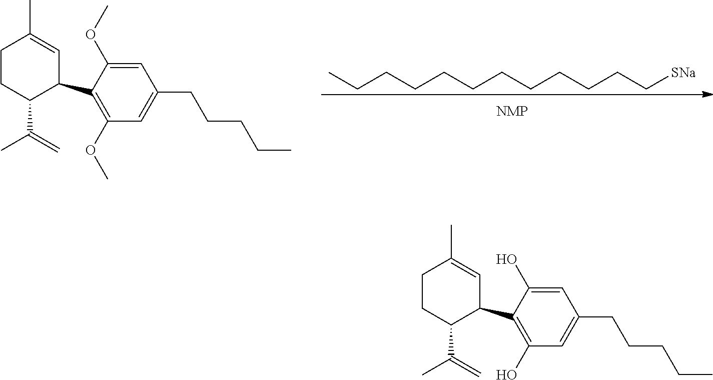

Sodium dodecylthiolate (494 mg, 2.2 mmol) was added to a solution of 1,3-dimethoxy-2-((1R,6R)-3-methyl-6-(prop-1-en-2-yl)cyclohex-2-enyl)-5-pentyl-benzene (250 mg, 0.73 mmol) in NMP (5 ml) and the mixture heated at 160° C. for 6 hours under argon. It was cooled to room temperature and neutralized with dilute sulfuric acid. The mixture was extracted with ethyl acetate (3×10 ml) and the combined organic layer was dried (MgSO₄), filtered and evaporated to dryness. The residue was purified by flash chromatography. Yield=195 mg.

### Example 92. Reaction of 1,3-dimethoxy-2-((1R,6R)-3-methyl-6-(prop-1-en-2-yl)cyclohex-2-enyl)-5-propylbenzene with Sodium Dodecylthiolate

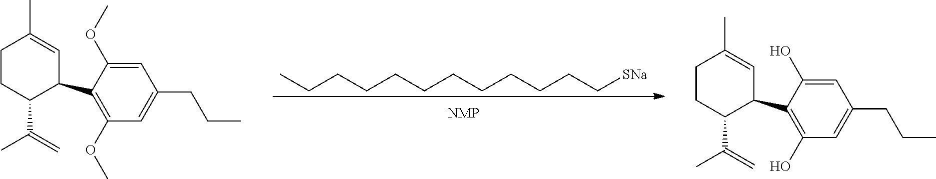

Sodium dodecylthiolate (494 mg, 2.2 mmol) was added to a solution of 1,3-dimethoxy-2-((1R,6R)-3-methyl-6-(prop-1-en-2-yl)cyclohex-2-enyl)-5-pentyl-benzene (250 mg, 0.79 mmol) in NMP (5 ml) and the mixture heated at 160° C. for 6 hours under argon. It was cooled to room temperature and neutralized with dilute sulfuric acid. The mixture was extracted with ethyl acetate (3×10 ml) and the combined organic layer was dried (MgSO₄), filtered and evaporated to dryness. The residue was purified by flash chromatography. Yield=182 mg.

### Example 93. Reaction of 1,3-dimethoxy-2-((1R,6R)-3-methyl-6-(prop-1-en-2-yl)cyclohex-2-enyl)-5-pentylbenzene with Methylmagnesium Iodide

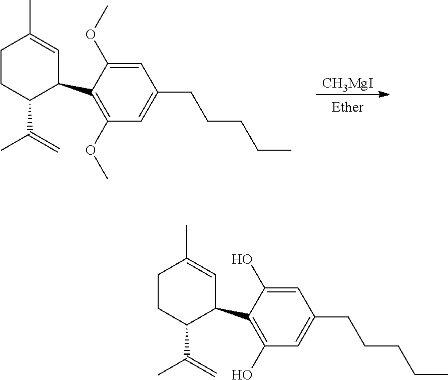 [SMILES: C=C(C)[C@@H]1CCC(C)=C[C@H]1c1c(OC)cc(CCCCC)cc1OC ; "CH3MgI" ; "Ether" ; C=C(C)[C@@H]1CCC(C)=C[C@H]1c1c(O)cc(CCCCC)cc1O]

A solution of methylmagnesium iodide (5.25 ml of a 3.0 M solution in diethyl ether, 15.8 mmol) was added to 1,3-dimethoxy-2-((1R,6R)-3-methyl-6-(prop-1-en-2-yl)cyclohex-2-enyl)-5-pentylbenzene (1.35 g, 3.94 mmol) and the mixture stirred for 30 minutes at room temperature under argon. It was then heated to 160° C. slowly under reduced pressure. It was heated at 160° C. for 3 hours under vacuum. The reaction was cooled to room temperature and ether added, followed by ammonium chloride solution (slowly). The phases were separated and the aqueous layer extracted with ether. The combined organic layers were dried (MgSO₄), filtered and evaporated to dryness. The residue was purified by flash chromatography. Yield=0.94 g.

### Example 94. Reaction of 3,5-dihydroxy-4-((1R,6R)-3-methyl-6-(prop-1-en-2-yl)cyclohex-2-enyl)phenyl Trifluoromethanesulfonate with n-Pentylmagnesium Bromide Using bis(diphenylphosphino)ethane]nickel(II) Chloride as Catalyst

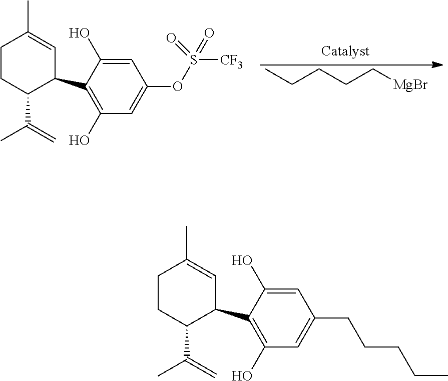

A solution of n-pentylmagnesium bromide (1.0 ml of a 1.0 M solution in diethyl ether, 1.0 mmol) was added to a mixture of 3,5-dihydroxy-4-((1R,6R)-3-methyl-6-(prop-1-en-2-yl)cyclohex-2-enyl)phenyl trifluoromethanesulfonate (100 mg, 0.25 mmol), NiCl₂(dppe) (13 mg, 0.025 mmol, 10%) and K₃PO₄ (54 mg, 0.24 mmol) and the mixture stirred at room temperature for 20 hours under argon. It was cooled to room temperature and quenched with ammonium chloride solution and diethyl ether added. The phases were separated and the organic layer was dried (MgSO₄), filtered and evaporated to dryness. The NMR spectrum of the residue showed 22% conversion of the substrate to the product.

### Example 95. Preparation of (6aR,10aR)-6,6,9-trimethyl-3-pentyl-6a,7,10,10a-tetrahydro-6H-benzo[c]chromen-1-ol (Δ⁸-tetrahydrocannabinol)

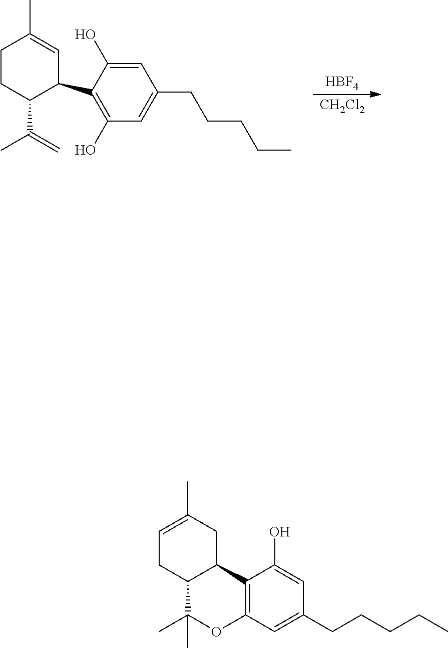 [SMILES: C=C(C)[C@@H]1CCC(C)=C[C@H]1c1c(O)cc(CCCCC)cc1O ; "HBF4" ; "CH2Cl2" ; CCCCCc1cc(O)c2c(c1)OC(C)(C)[C@@H]1CC=C(C)C[C@@H]21]

A solution of HBF₄.Et₂O (40 mg, 0.25 mmol) was added to a solution of 2-((1R,6R)-3-methyl-6-(prop-1-en-2-yl)cyclohex-2-enyl)-5-pentylbenzene-1,3-diol (1.0 g, 3.18 mmol) in dichloromethane (10 ml) and the mixture was stirred at room temperature for 16 hours. The reaction was quenched with water and the phases separated. The organic layer was washed with NaHCO₃ solution, dried (MgSO₄), filtered and evaporated to dryness to give the product as a yellow resin. Yield=0.84 g.

### Example 96. Preparation of (6aR,10aR)-3-heptyl-6,6,9-trimethyl-6a,7,10,10a-tetrahydro-6H-benzo[c]chromen-1-ol (Δ⁸-tetrahydrocannabiphorol)

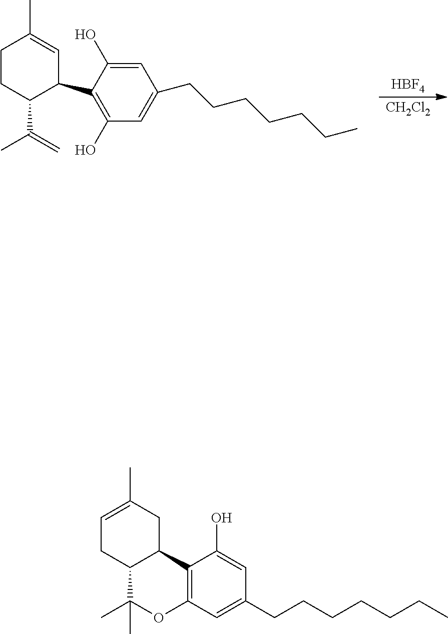

This was prepared according to the procedure outlined in Example 95 and using 5-heptyl-2-((1R,6R)-3-methyl-6-(prop-1-en-2-yl)cyclohex-2-enyl)benzene-1,3-diol. The product was isolated as a yellow resin.

### Example 97. Preparation of (6aR,10aR)-6,6,9-trimethyl-3-phenethyl-6a,7,10,10a-tetrahydro-6H-benzo[c]chromen-1-ol

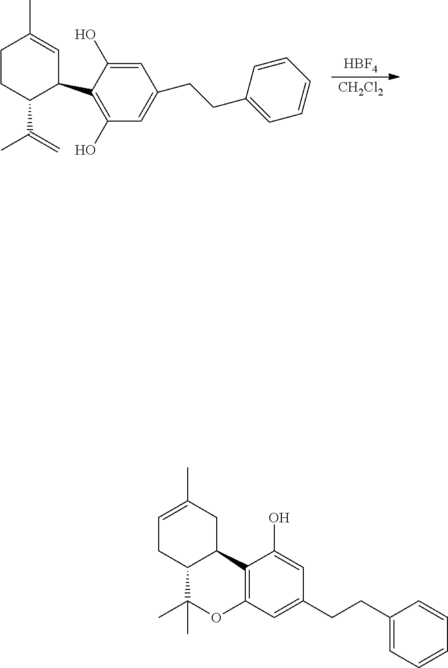

This was prepared according to the procedure outlined in Example 95 and using 2-((1R,6R)-3-methyl-6-(prop-1-en-2-yl)cyclohex-2-enyl)-5-phenethylbenzene-1,3-diol. The product was isolated as a yellow resin.

### Example 98. Preparation of 5-bromo-1,3-dimethoxy-2-((1R,6R)-3-methyl-6-(prop-1-en-2-yl)cyclohex-2-enyl)benzene

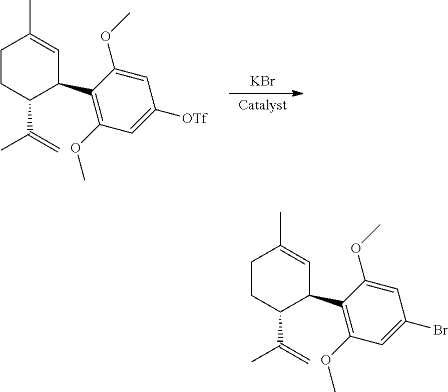 [SMILES: C=C(C)[C@@H]1CCC(C)=C[C@H]1c1c(OC)cc(OS(=O)(=O)C(F)(F)F)cc1OC ; "KBr" ; "Catalyst" ; C=C(C)[C@@H]1CCC(C)=C[C@H]1c1c(OC)cc(Br)cc1OC]

A solution of tBuBrettPhos (4.8 mg, 0.01 mmol) and Pd₂(dba)₃ (4.6 mg, 0.005 mmol) in Dioxane (5 ml) was added to a mixture of 3,5-dimethoxy-4-((1R,6R)-3-methyl-6-(prop-1-en-2-yl)cyclohex-2-enyl)phenyl trifluoromethanesulfonate (210 mg, 0.5 mmol), KBr (120 mg, 1.0 mmol) and KF (15 mg, 0.25 mmol) under argon and the mixture was stirred vigorously at 120° C. for 16 hours. It was cooled to room temperature and filtered through a pad of silica gel and concentrated under reduced pressure. The product was purified by silica gel chromatography. Yield=105 mg.

### Example 99. Preparation of (5-bromo-2-((1R,6R)-3-methyl-6-(prop-1-en-2-yl)cyclohex-2-enyl)-1,3-phenylene)bis(oxy)bis(trimethylsilane)

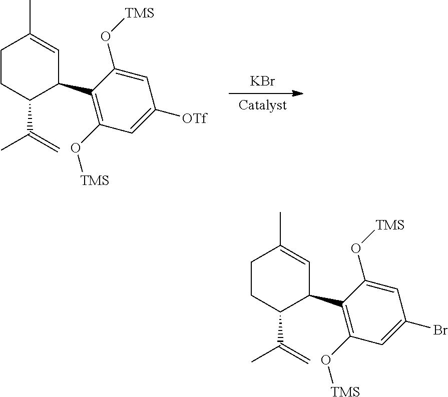 [SMILES: C=C(C)[C@@H]1CCC(C)=C[C@H]1c1c(O[Si](C)(C)C)cc(OS(=O)(=O)C(F)(F)F)cc1O[Si](C)(C)C ; "KBr" ; "Catalyst" ; C=C(C)[C@@H]1CCC(C)=C[C@H]1c1c(O[Si](C)(C)C)cc(Br)cc1O[Si](C)(C)C]

A solution of tBuBrettPhos (19 mg, 0.04 mmol) and Pd₂(dba)₃ (18 mg, 0.02 mmol) in Dioxane (5 ml) was added to a mixture of 4-((1R,6R)-3-methyl-6-(prop-1-en-2-yl)cyclohex-2-enyl)-3,5-bis(trimethylsilyloxy)phenyl trifluoromethanesulfonate (540 mg, 1.0 mmol) and KBr (240 mg, 2.0 mmol) under argon and the mixture was stirred vigorously at 120° C. for 20 hours. It was cooled to room temperature and filtered through a pad of silica gel and concentrated under reduced pressure. The product was purified by silica gel chromatography. Yield=182 mg.

### Example 100. Reaction of 5-bromo-1,3-dimethoxy-2-((1R,6R)-3-methyl-6-(prop-1-en-2-yl)cyclohex-2-enyl)benzene with n-Pentylzinc Bromide

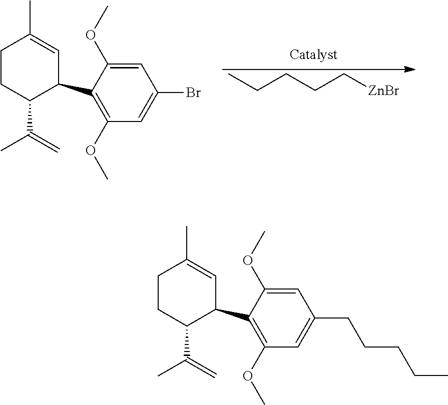

A solution of n-pentylzinc bromide (0.67 ml of a 0.5 M solution in THF, 0.34 mmol) was added to a mixture of 5-bromo-1,3-dimethoxy-2-((1R,6R)-3-methyl-6-(prop-1-en-2-yl)cyclohex-2-enyl)benzene (100 mg, 0.28 mmol) and PdCl₂(dppf) (5 mg, 0.007 mmol) and the mixture stirred at room temperature for 6 hours under argon. It was quenched with ammonium chloride solution and diethyl ether added. The phases were separated and the organic layer was dried (MgSO₄), filtered and evaporated to dryness. The NMR spectrum of the residue shows 100% conversion of the substrate to the product. Flash chromatography using hexanes/ethyl acetate yielded the product as a pale yellow oil. Yield=92 mg.

### Example 101. Preparation of 3,5-dimethoxy-4-((1R,6R)-3-methyl-6-(prop-1-en-2-yl)cyclohex-2-enyl)phenylboronic Acid

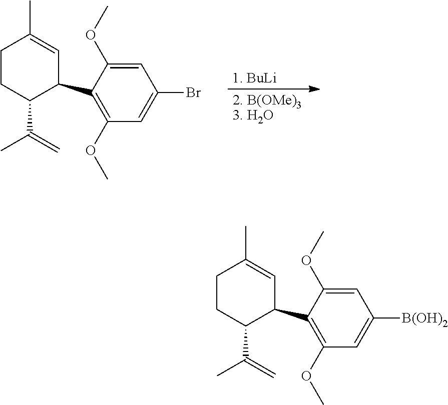

A solution of 5-bromo-1,3-dimethoxy-2-((1R,6R)-3-methyl-6-(prop-1-en-2-yl)cyclohex-2-enyl)benzene (100 mg, 0.28 mmol) in THF (5 ml) was cooled to −70° C. and butyllithium (0.2 ml of a 1.6 M solution in hexanes, 0.32 mmol) added. The mixture was stirred for 1 hour under argon, and trimethylborate (35 mg, 0.34 mmol) added. The mixture was then allowed to warm to room temperature and stirred for 4 hours under argon. It was quenched with ammonium chloride solution and stirred overnight. Ethyl acetate was added and the phases were separated. The organic layer was dried (MgSO₄), filtered and evaporated to dryness. The residue was recrystallized from ethyl acetate and hexanes. Yield=82 mg.

### Example 102. Preparation of (3,5-dimethoxy-4-((1R,6R)-3-methyl-6-(prop-1-en-2-yl)cyclohex-2-enyl)phenyl)magnesium Bromide

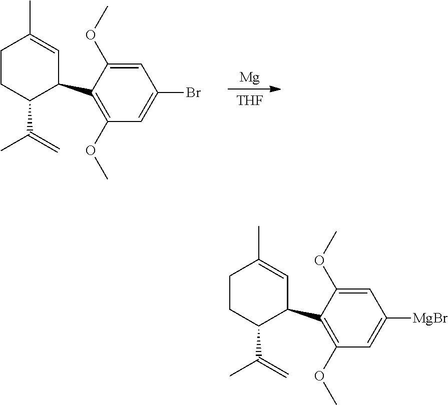

A solution of 5-bromo-1,3-dimethoxy-2-((1R,6R)-3-methyl-6-(prop-1-en-2-yl)cyclohex-2-enyl)benzene (485 mg, 1.38 mmol) in THF (5 ml) was added to magnesium turnings (40 mg, 1.7 mmol) and the mixture heated at 60° C. for 2 hours.

### Example 103. Reaction of (3,5-dimethoxy-4-((1R,6R)-3-methyl-6-(prop-1-en-2-yl)cyclohex-2-enyl)phenyl)magnesium Bromide with n-Pentyl Bromide

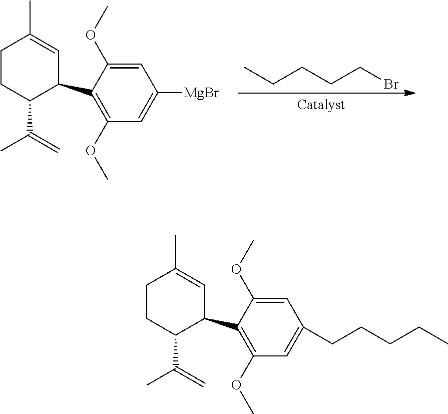

The solution of (3,5-dimethoxy-4-((1R,6R)-3-methyl-6-(prop-1-en-2-yl)cyclohex-2-enyl)phenyl)magnesium bromide from Example 102 (1.38 mmol) was added to a mixture of ZnBr₂ (622 mg, 2.76 mmol) and LiBr (240 mg, 2.76 mmol) in THF (40 ml) and the suspension was stirred for 30 minutes under argon. A solution of n-pentyl bromide (250 mg, 1.66 mmol) and PdCl₂(dppf) (14 mg, 0.019 mmol) in THF (5 ml) was added and the mixture was stirred at room temperature for 4 hours under argon. Water (10 ml) was added followed by dilute H₂SO₄ (1.0 ml) and the mixture stirred at room temperature for 1 hour. The phases were separated and the organic layer was dried (MgSO₄), filtered and evaporated to dryness. The residue was dissolved in hexanes and filtered through a short pad of silica gel. The silica was washed with hexanes and the combined filtrate was evaporated to dryness to give a pale yellow oil. Yield=435 mg.

### Example 104. Purification of CANNABIDIOL

Crude cannabidiol (10.0 g) was dissolved in isooctane (40 ml) and heated to 40° C. The solution was cooled to 32° C., seeded with cannabidiol crystals and stirred at 32° C. for 1 hour. The suspension was slowly cooled to −20° C. and stirred for 2 hours. The crystals were filtered and washed with cold (−20° C.) isooctane (40 ml). The product was dried under vacuum to give pure and crystalline cannabidiol. Yield=9.4 g.

While the foregoing invention has been described in some detail for purposes of clarity and understanding, it will be appreciated by one skilled in the art, from a reading of the disclosure that various changes in form and detail can be made without departing from the true scope of the invention in the appended claims.

All publications, patents, and patent applications are herein incorporated by reference in their entirety to the same extent as if each individual publication, patent or patent application was specifically and individually indicated to be incorporated by reference in its entirety.

1. A compound of Formula (I): 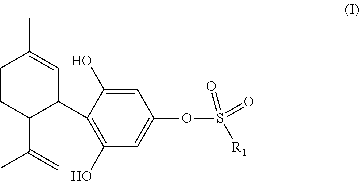
R₁ represents a hydrogen atom, —OR^{c}, —NR^{c}₂, fluoro-substituted-(C₁-C₂₀)-alkyl, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, or a (C₅-C₁₄)-heteroaryl group, wherein the latter 6 groups are each optionally substituted with one or more halogen atoms, —(C₁-C₂₀)-alkyl, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl;
and any stereoisomers or acceptable salts thereof.

2. The compound of Formula (I) of claim 1, wherein R₁ represents a hydrogen atom, fluoro-substituted-(C₁-C₂₀)-alkyl, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, a (C₅-C₁₄)-heteroaryl group, wherein the latter 6 groups are each optionally substituted with one or more halogen atoms, a —(C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

3. The compound of Formula (I) of claim 1, wherein R₁ represents a hydrogen atom, fluoro-substituted-(C₁-C₁₀)-alkyl, a (C₁-C₁₀)-alkyl group, a (C₂-C₁₀)-alkenyl group, a (C₂-C₁₀)-alkynyl group, a (C₃-C₁₀)-cycloalkyl group, a (C₆-C₁₀)-aryl group, a (C₅-C₁₀)-heteroaryl group, wherein the latter 6 groups are each optionally substituted with one or more halogen atoms (F, Cl, Br or I), —(C₁-C₂₀)-alkyl, a (C₂-C₂₀)-alkenyl group, or a (C₂-C₂₀)-alkynyl group.

4. The compound of Formula (I) of claim 1, wherein R₁ represents a hydrogen atom, fluoro-substituted-(C₁-C₆)-alkyl, a (C₁-C₆)-alkyl group, a (C₂-C₆)-alkenyl group, a (C₂-C₆)-alkynyl group, a (C₃-C₆)-cycloalkyl group, a (C₆)-aryl group, a (C₅-C₆)-heteroaryl group, wherein the latter 6 groups are each optionally substituted with one or more halogen atoms, or —(C₁-C₂₀)-alkyl.

5. The compound of Formula (I) of claim 1, wherein R₁ represents a hydrogen atom, fluoro-substituted-(C₁-C₆)-alkyl, a (C₁-C₆)-alkyl group, a (C₂-C₆)-alkenyl group, a (C₂-C₆)-alkynyl group, a (C₃-C₆)-cycloalkyl group, a (C₆)-aryl group, a (C₅-C₆)-heteroaryl group, wherein the latter 6 groups are each optionally substituted with one or more halogen atoms, or —(C₁-C₂₀)-alkyl.

6. The compound of Formula (I) of claim 1, wherein R₁ represents a hydrogen atom, —CF₃,

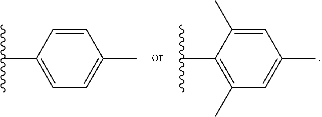

7. The compound of Formula (I) of claim 1, wherein the compound of Formula (I) is

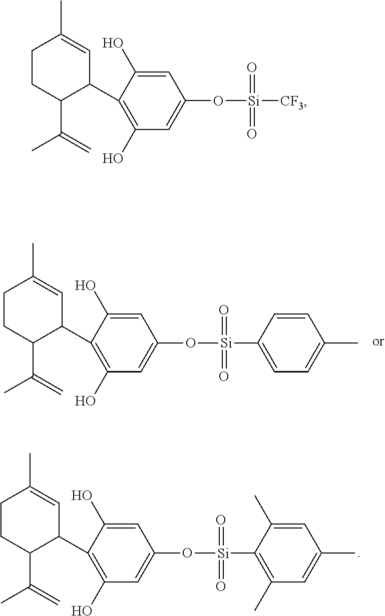

8. A compound of Formula (II): 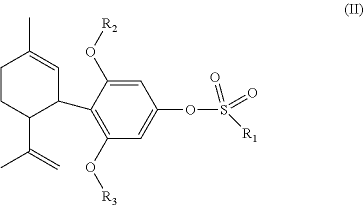
wherein R₁ is a hydrogen atom, —OR^{c}, —NR^{c}₂, fluoro-substituted-(C₁-C₂₀)-alkyl, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, or a (C₅-C₁₄)-heteroaryl group, wherein the latter 6 groups are each optionally substituted with one or more halogen atoms, —(C₁-C₂₀)-alkyl, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl;
R₂ and R₃ independently or simultaneously represent a (C₁-C₂₀)-alkyl group, a (C₂-C₂₀)-alkenyl group, a (C₂-C₂₀)-alkynyl group, a (C₃-C₂₀)-cycloalkyl group, a —Si[(C₁-C₂₀)-alkyl]₃ group, a (C₆-C₁₄)-aryl group, or a (C₅-C₁₄)-heteroaryl group, or an acyl group —C(═O)—R′, wherein R′ is a (C₁-C₂₀)-alkyl group, wherein each group is each optionally substituted with one or more halogen atoms (F, Cl, Br or I), a —(C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl, and
wherein one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more halogen (F, Cl, Br or I), or a —(C₁-C₂₀)-alkyl groups;
and any stereoisomers or acceptable salts thereof.

9. The compound of Formula (II) of claim 8, wherein R₂ and R₃ independently or simultaneously represent a (C₁-C₁₀)-alkyl group, a (C₂-C₁₀)-alkenyl group, a (C₂-C₁₀)-alkynyl group, a (C₃-C₁₀)-cycloalkyl group, a —Si[(C₁-C₁₀)-alkyl]₃ group, a (C₆-C₁₀)-aryl group, or a (C₅-C₁₀)-heteroaryl group, or an acyl group —C(═O)—R′, wherein R′ is a (C₁-C₁₀)-alkyl group, wherein each group is each optionally substituted with one or more halogen atoms (F, Cl, Br or I), a —(C₁-C₁₀)-alkyl group, a (C₂-C₁₀)-alkenyl group, a (C₂-C₁₀)-alkynyl group, —OR^{d}, or —NR^{d}₂, wherein R^{c} and R^{d} are independently or simultaneously hydrogen, (C₁-C₁₀)-alkyl, (C₂-C₁₀)-alkenyl, or (C₂-C₁₀)-alkynyl, and
wherein one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more halogen (F, Cl, Br or I), or a —(C₁-C₁₀)-alkyl groups.

10. The compound of claim 8, wherein R₂ and R₃ independently or simultaneously represent a (C₁-C₆)-alkyl group, a (C₂-C₆)-alkenyl group, a (C₂-C₆)-alkynyl group, a (C₃-C₆)-cycloalkyl group, a —Si[(C₁-C₆)-alkyl]₃ group, a phenyl group, or a (C₅-C₆)-heteroaryl group, or an acyl group —C(═O)—R′, wherein R′ is a (C₁-C₆)-alkyl group, wherein each group is each optionally substituted with one or more halogen atoms (F, Cl, Br or I), a —(C₁-C₆)-alkyl group, a (C₂-C₆)-alkenyl group, a (C₂-C₆)-alkynyl group, —OR^{d}, or —NR^{d}₂, wherein R^{c} and R^{d} are independently or simultaneously hydrogen, (C₁-C₆)-alkyl, (C₂-C₆)-alkenyl, or (C₂-C₆)-alkynyl, and
wherein one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more halogen (F, Cl, Br or I), or a —(C₁-C₆)-alkyl groups.

11. The compound claim 8, wherein R₂ and R₃ independently or simultaneously represent a (C₁-C₆)-alkyl group, a —Si[(C₁-C₆)-alkyl]₃ group, or a phenyl group.

12. The compound claim 8, wherein R₂ and R₃ independently or simultaneously represent a —Si[(C₁-C₆)-alkyl]₃ group.

13. The compound claim 8, wherein R₂ and R₃ independently or simultaneously represent a —Si[(C₁-C₃)-alkyl]₃ group.

14. The compound claim 8, wherein R₂ and R₃ represent a —Si(CH₃)₃ group.

15. A process for the preparation of compounds of Formula (III) or Formula (VI): 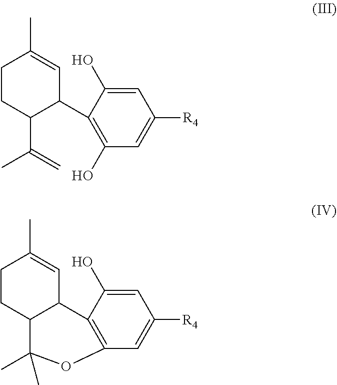
wherein R₂ and R₃
independently or simultaneously represent a (C₁-C₂₀)-alkyl group, a (C₂-C₂₀)-alkenyl group, a (C₂-C₂₀)-alkynyl group, a (C₃-C₂₀)-cycloalkyl group, a —Si[(C₁-C₂₀)-alkyl]₃ group, a (C₆-C₁₄)-aryl group, or a (C₅-C₁₄)-heteroaryl group, or an acyl group —C(═O)—R′, wherein R′ is a (C₁-C₂₀)-alkyl group, wherein each group is each optionally substituted with one or more halogen atoms (F, Cl, Br or I), a —(C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl, and
wherein one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more halogen (F, Cl, Br or I), or a —(C₁-C₂₀)-alkyl groups;
the process comprising reacting a compound of Formula (I) as defined in claim 1:
(i) a boron containing compound which is R₄—B(OH)₂, R₄—B(OR)₂ or R₄—BF₃K, where R is H, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₄)-aryl group;
(ii) a Grignard compound which is R₄—MgX; or
(iii) a zinc compound which is R₄—ZnX.
wherein X is a halogen atom and;
R₄ represents a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, wherein the latter 5 groups are each optionally substituted with one or more halogen atoms (F, Cl, Br or I), —(C₁-C₂₀)-alkyl, a (C₂-C₂₀)-alkenyl group, a (C₂-C₂₀)-alkynyl group, (C₆-C₁₄)-aryl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

16. The process of claim 15, wherein a compound of Formula (III), Formula (IV), Formula (V) or Formula (VI) is a pure isomer or a mixture of isomers.

17. (canceled)

18. (canceled)

19. (canceled)

20. (canceled)

21. (canceled)

22. A compound of Formula (X) 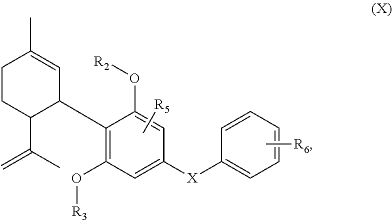
R₂ and R₃ independently or simultaneously represent a (C₁-C₂₀)-alkyl group, a (C₂-C₂₀)-alkenyl group, a (C₂-C₂₀)-alkynyl group, a (C₃-C₂₀-cycloalkyl group, a —Si[(C₁-C₂₀)-alkyl]₃ group, a (C₆-C₁₄)-aryl group, or a (C₅-C₁₄)-heteroaryl group, or an acyl group —C(═O)—R′, wherein R′ is a (C₁-C₂₀)-alkyl group, wherein each group is each optionally substituted with one or more halogen atoms (F, Cl, Br or I), a —(C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl, and
wherein one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more halogen (F, Cl, Br or I), or a —(C₁-C₂₀)-alkyl groups;
R₅ and R₆ are one or more substitutents which are hydrogen, halo, —OR^{c}, —NR^{c}₂, carboxylates (—COOR, where R is H or (C₁-C₆)-alkyl), phosphates, sulfates, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, or a (C₅-C₁₄)-heteroaryl group, wherein R^{c} and R^{d} are independently or simultaneously hydrogen, (C₁-C₂₀)-alkyl, (C₂-C₂₀)-alkenyl, or (C₂-C₂₀)-alkynyl;
X is (C₁-C₁₀-alkylene) or (C₂-C₁₀-alkenylene);
and all stereoisomers, and salts thereof.

23. The compound of Formula (II) of claim 8, wherein R₁ represents a hydrogen atom, fluoro-substituted-(C₁-C₁₀)-alkyl, a (C₁-C₁₀)-alkyl group, a (C₂-C₁₀)-alkenyl group, a (C₂-C₁₀)-alkynyl group, a (C₃-C₁₀)-cycloalkyl group, a (C₆-C₁₀)-aryl group, a (C₅-C₁₀)-heteroaryl group, wherein the latter 6 groups are each optionally substituted with one or more halogen atoms (F, Cl, Br or I), —(C₁-C₂₀)-alkyl, a (C₂-C₂₀)-alkenyl group, or a (C₂-C₂₀)-alkynyl group.

24. The compound of Formula (II) of claim 8, wherein R₁ represents a hydrogen atom, fluoro-substituted-(C₁-C₆)-alkyl, a (C₁-C₆)-alkyl group, a (C₂-C₆)-alkenyl group, a (C₂-C₆)-alkynyl group, a (C₃-C₆)-cycloalkyl group, a (C₆)-aryl group, a (C₅-C₆)-heteroaryl group, wherein the latter 6 groups are each optionally substituted with one or more halogen atoms, or —(C₁-C₂₀)-alkyl.

25. The compound of Formula (II) of claim 8, wherein R₁ represents a hydrogen atom, fluoro-substituted-(C₁-C₆)-alkyl, a (C₁-C₆)-alkyl group, or a phenyl group, wherein the latter 2 groups are each optionally substituted with one or more halogen atoms, or —(C₁-C₁₀)-alkyl.

26. A process for the preparation of compounds of Formula (V) or Formula (VI): 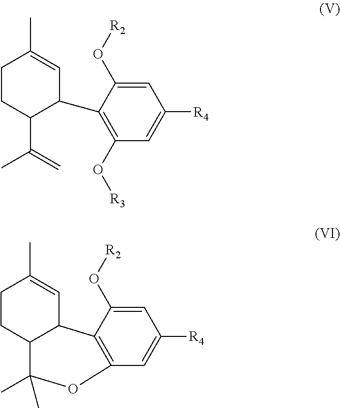
the process comprising reacting a compound of Formula (II) as defined in claim 8 with:
(i) a boron containing compound which is R₄—B(OH)₂, R₄—B(OR)₂ or R₄—BF₃K, where R is H, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₄)-aryl group;
(ii) a Grignard compound which is R₄—MgX; or
(iii) a zinc compound which is R₄—ZnX.
wherein X is a halogen atom; and
R₂ and R₃ independently or simultaneously represent a (C₁-C₂₀)-alkyl group, a (C₂-C₂₀)-alkenyl group, a (C₂-C₂₀)-alkynyl group, a (C₃-C₂₀)-cycloalkyl group, a —Si[(C₁-C₂₀)-alkyl]₃ group, a (C₆-C₁₄)-aryl group, or a (C₅-C₁₄)-heteroaryl group, or an acyl group —C(═O)—R′, wherein R′ is a (C₁-C₂₀)-alkyl group, wherein each group is each optionally substituted with one or more halogen atoms (F, Cl, Br or I), a —(C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl, and
wherein one or more of the carbon atoms in the alkyl, alkenyl, alkynyl, cycloalkyl, aryl, heteroaryl or acyl groups of R₂ and/or R₃ is optionally replaced with a heteroatom selected from the group consisting of O, S, N, P and Si, which, where possible, is optionally substituted with one or more halogen (F, Cl, Br or I), or a —(C₁-C₂₀)-alkyl groups;
R₄ represents a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, wherein the latter 5 groups are each optionally substituted with one or more halogen atoms (F, Cl, Br or I), —(C₁-C₂₀)-alkyl, a (C₂-C₂₀)-alkenyl group, a (C₂-C₂₀)-alkynyl group, (C₆-C₁₄)-aryl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.
